(19) United States
(12) Patent Application Publication
(10) Pub. No.: US 2010/0076503 A1
(43) Pub. Date: Mar. 25, 2010

(54) BONE IMPLANT

(75) Inventors: Mordechay Beyar, Herzlia Pituach (IL); Oren Globerman, Herzlia Pituach (IL); Elad Einav, Zikhron-Yaakov (IL)

Correspondence Address: MARTIN D. MOYNIHAN d/b/a PRTSI, INC., P.O. BOX 16446, ARLINGTON, VA, 22215, US

(73) Assignee: N.M.B. Medical Applications Ltd, Herzlia (IL)

(21) Appl. No.: 12/526,266

(22) PCT Filed: Feb. 7, 2008
(86) PCT No.: PCT/IL08/00171

§ 371 Date: Nov. 17, 2009

(30) Foreign Application Priority Data
Feb. 7, 2007 (IL) 181211
Apr. 26, 2007 (IL) 042607

(51) Int. Cl. A61B 17/56

(52) U.S. Cl. 606/86 R

(57) ABSTRACT

A method of long bone strengthening and a composite implant for such strengthening. Also disclosed is a kit for building a composite implant in-situ in long bones. In an exemplary embodiment of the invention, the implant comprises a plurality of rigid tensile rods, in matrix of cement and surrounded by a partially porous bag.

### RELATED APPLICATIONS

The present application claims priority from IL Patent Application No. 181211 filed on Feb. 7, 2007, and from IL Patent application No. 182821 filed 26 Apr. 2007 the disclosures of which are incorporated herein by reference.

### FIELD OF THE INVENTION

The present invention relates to methods and devices for strengthening of long bones, for example effective for preventing fractures of bones.

### BACKGROUND OF THE INVENTION

Hip fractures are a leading (indirect) cause of death in the elderly. Typically, Osteoporosis, in which the inner trabecular structure of the bone is destroyed, underlies the hip fracture. Furthermore, it is known that a fracture in one hip is a reliable indicator of danger of fracture in the other hip.

A typical bone structure is an outside of hard cortical bone and an inside formed of softer bone, such as trabecular bone and/or marrow or other non-structural tissue. Various blood vessels are also provided in bone.

U.S. Pat. No. 6,679,890, the disclosure of which is incorporated herein by reference, suggests hollowing out a portion of a hip and injecting cement therein and then inserting a rigid implant.

U.S. Pat. No. 4,755,184, the disclosure of which is incorporated herein by reference, describes a bone implant comprising a porous resorbable bag filled with cement.

U.S. Pat. No. 5,827,289, the disclosure of which is incorporated herein by reference, suggests drilling a tunnel in a bone, compacting bone surrounding the tunnel using a balloon and then filling the tunnel with cement.

U.S. Pat. No. 6,425,923, the disclosure of which is incorporated herein by reference, discloses a bag to be implanted in a bone and filled with cement.

J Bone Joint Surg [Br] 2005; 87-B:1320-7, and J Bone Miner Res 2000; 15:721-739 the disclosures of which are incorporated herein by reference discusses the hip fracture problem in general.

J Vasc Intery Radiol 2004; 15:121-126, the disclosure of which is incorporated herein by reference, discusses various bone cement compositions.

### SUMMARY OF THE INVENTION

A broad aspect of some embodiments of the invention relates to treating and/or preventing bone fractures, especially in long bones. In some embodiments, a composite implant is constructed in situ in the cortical bone and/or medullar portion of the bone. In some embodiments, a metallic (or other) nail or screw is used.

In an exemplary embodiment of the invention, the method is applied as a preventive measure, even though a bone to be treated is not indicated as being fractured.

In an exemplary embodiment of the invention, a composite implant is used which includes both one or more tensile elements and a compressive element, such as hardened bone cement or an adhesive material. In an exemplary embodiment of the invention, the tensile elements are rods. In an exemplary embodiment of the invention, a bag is provided enclosing most of the implant and/or the rods. Optionally, the bag acts as a (optionally single) tensile element.

In an exemplary embodiment of the invention, the implant is constructed in situ via an aperture in the bone considerably smaller in diameter than the final implant. In an exemplary embodiment of the invention, a plurality of rods or other tensile elements are inserted through the aperture to lie side by side, or are otherwise adjacent, in the implant.

In an exemplary embodiment of the invention, the implant is formulated and constructed to match needs of a specific patient, for example, as estimated or as measured (e.g., pre-treatment or during treatment). Optionally, a suggested formulation is provided by a table or a circuitry based calculation

In an exemplary embodiment of the invention, the implant is configured to have properties (e.g., one or more of density and elastic modulus), similar to that of surrounding bone, for example, trabecular and/or cortical bone.

In an exemplary embodiment of the invention, optionally, the whole implant acts mainly as a strengthening element to the treated bone and not mainly as a fixating element as in known bone implants, while maintaining at least most natural strains distributed in and/or through the treated bone. In an exemplary embodiment of the invention, an implant for strengthening differs from an implant for fixating, by providing substantially equal engagement of cortical and/or trabecular bone along its length (optionally excepting the cortical entrance). In an exemplary embodiment of the invention, the bone engagement per unit length at a distal end of the implant is less than a factor of 4, 3, 2, 1.5 or 1.3 greater than engagement per unit length along any tubular section of 30% in length of the implant. Optionally or alternatively, at most 60%, 40%, 30%, 20% of engagement is provided by a distal end of the implant (e.g., a widening thereof).

In an exemplary embodiment of the invention, the implant is positioned so that it leans on the cortex of the bone at least two locations, for example, near an entrance to the bone and about midway along the implant. Alternatively, the implant leans on cortical bone at only one location, or even none.

In an exemplary embodiment of the invention, an implant constructing kit is provided, which includes a tensile element carrier adapted to carry tensile elements in to a bone void. Optionally, there is included a bag holder which advances a bag (and optionally holds it) into the void. Optionally or alternatively, there is provided a bone drill optionally adapted to form a cavity in bone greater than the diameter of the aperture.

In an exemplary embodiment of the invention, a guide-wire drill is used, in which a shaft-like element has a drilling tip adapted to penetrate cortical bone and also suitable to attach at a distal side to a mechanical rotational source (e.g., a drill handle), while having a diameter suitable for use as a guide wire for passing cannula and/or other tools over the drill, during a procedure.

In an exemplary embodiment of the invention, the drill includes a side-extending element which is adapted to form a void in trabecular bone and/or remove cortical bone, when suitably manipulated.

In some embodiments, flexible elements are used instead of or in addition to rods, for example, yarn elements. In some embodiments, no cement is used in the implant.

An aspect of some embodiments of the invention relates to strengthening a bone using an implant comprising a mixture of hardening material and tensile elements which contribute significantly to the fracture and/or cracking resistance of the bone and/or implant. In an exemplary embodiment of the invention, the cement is disposed inside a container which acts as a tensile element. In an exemplary embodiment of the invention, the tensile elements and cement constitute a composite material. In an exemplary embodiment of the invention, the tensile elements are made of a composite or non-composite material, and they are inserted into the bone, optionally in combination with cement. Optionally or alternatively, the rods are inserted into a container. In an exemplary embodiment of the invention, the container provides only tensile strength. In an exemplary embodiment of the invention, the combination of cement and tensile elements provides stiffness and/or bending resistance equivalent to bone (or better). Optionally, the implant is designed to resist breakage at a degree of bending below that which will cause the bone to break.

Optionally, the implant is indicated and/or used for osteroporotic bone, metastatic bone, or other bone pathology that may affect the strength of the bone. Optionally, the implant is designed to allow the bone to receive stresses, for example, stresses similar to natural bone stresses of that bone. Optionally, the implant is used to fixate the bone while the bone heals, following bone breakage, optionally for non-separating fractures.

In an exemplary embodiment of the invention, when used for fixating, a widening of the implant (e.g., cement and/or bag) is provided at either end and/or distal end.

In an exemplary embodiment of the invention, when used for fixating, the implant in inserted and filled with cement mainly at its distal side. This cement is allowed to harden and then the implant is retracted, optionally to reduce a fracture and/or pre-tense the bone and then the rest of the cement is injected into the implant.

In an exemplary embodiment of the invention, the implant includes at least 20%, 30%, 45%, 60%, 80% or more or intermediate percentages by volume of longitudinal tensile elements.

In an exemplary embodiment of the invention, the bone being strengthened is a long bone, such as a leg bone (e.g., femur, tibia, fibula) an arm bone (e.g. humerus, ulna, radius), foot and hand phalanges or a clavicle, and the implant is elongate, for example, with a width-length ratio of at least 1:3 or 1:4. In an exemplary embodiment of the invention, an optional length of implant for the femur is between 25 mm and 500 mm, optionally between 50-100 mm, with optional diameter of between 1 mm and 20 mm, optionally 10 mm.

In an exemplary embodiment of the invention, the implant comprises a flexible, substantially inelastic bag, optionally mesh, as a container, filled with a bone fixing material (e.g., provided as a fluid or paste and hardens to a solid, for example through a setting process). Typical such fixing materials include but are not limited to bone cement, such as PMMA, calcium phosphate, epoxy and/or kryptonite. In an exemplary embodiment of the invention, the bag comprises a tube closed off at a distal end thereof.

In an exemplary embodiment of the invention, the bag functions to provide tensile strength and/or hold together the cement and prevent cracking thereof. Optionally, some of the tensile behavior is provided by the bag and some by additional tensile elements. Optionally, some of the compressive behavior is provided by the cement and some by additional elements. Optionally 50% of the bending resistance of the implant is provided by the bag. Optionally or alternatively, 50% of the bending resistance provide by the cement (e.g., with bag providing tensile resistance and cement providing compressive resistance). In other embodiments, the bag provides between 10%-80% of the bending resistance. Alternatively the cement provides between 10%-80% of the bending resistance. Optionally, the implant can withstand forces of greater than 100 Kg, 200 Kg, 300 Kg, and/or impulses of greater than 100 Kg/sec, 200 Kg sec, 1000 Kg/sec or intermediate values, as applied differentially to either side of the implant and perpendicular thereto.

Optionally, the bag includes longitudinal fibers. Optionally, the fibers are configured to yield a small amount in the longitudinal direction. Alternatively or additionally, the bag includes circumferential fibers. Optionally, the bag is preformed to be curved. Alternatively or additionally, the bag is bent as part of an implantation process.

In an exemplary embodiment of the invention, the bag element contains a mesh having holes that are large enough to allow a small amount of cement to escape the bag as it is filled, for example, to provide inter-digitation with surrounding tissue such as trabecular tissue. Alternatively, cement does not emerge from the bag, as for example in cases where the mesh holes are too small with respect to cement viscosity and/or particle sizes. Optionally, the mesh is porous enough to allow air exit from the bag as it is filled.

In an exemplary embodiment of the invention, the bag is configured to allow cement leakage (the term seepage is also used herein) through it in some predefined parts, while it optionally does not permit (or permits reduced amount of) cement leakage in other parts. Optionally, the mesh holes are small enough so that the act of filling the bag causes the bag to expand with enough force to compact surrounding trabecular bone.

In an exemplary embodiment of the invention, the bag is formed (e.g., woven) in a manner which defines specific pores. Optionally, general pores are defined by the density of the weave, of longitudinal and circumferential fibers. In an exemplary embodiment of the invention, specific pores are formed by one or more circumferential fibers being folded back at a pore, rather than crossing the pore. Optionally, such a pore may have an axial width of one, two, three or more circumferential fibers. Optionally or alternatively, a similar arrangement is provided for longitudinal fibers.

Optionally, a bio-absorbable cement is used and the pores are selected so that bone can grow through them. Optionally, the mesh is also bio-degradable/bio-absorbable.

In some embodiments of the invention, the implant includes multiple layers of tensile elements, for example formed by providing a container within a container. Optionally, the containers are designed to maintain spacing between them, for example are concentrically disposed. Alternatively or additionally, an outer container is less permeable than an inner container.

In embodiments of the invention the implant is devoid of a container such as a bag.

In some embodiments of the invention, the implant comprises tensile elements, such as fibers or rods, embedded within the cement, in embodiments in addition to a container and in embodiments devoid of a container. Optionally, the rods are formed of carbon-PEEK composite, carbon-PEKK composite and/or carbon-PMMA composite.

In some embodiments, the container provides less than 10% of tensile strength of the implant. Optionally, the mesh prevents propagation of surface cracking.

In an exemplary embodiment of the invention, more than one implant is introduced into bone. In an exemplary embodiment of the invention, two containers are inserted into a proximal femur, filled with rods of composite material (tensile elements), and later filled with bone cement. In another embodiment, a plurality of holes are drilled in bone, optionally in cortical bone, and filled with cement and one or more tensile elements. In another embodiment, in a hip, two crossing channels are defined, one along femur and one along trochanter, such that an implant is formed along the two lines.

In an exemplary embodiment of the invention, the implant is used in bones in locations that experience forces addition to compression forces, for example, experiencing tension, bending, shear and/or rotation forces.

In an exemplary embodiment of the invention, the implant is not strong enough to withstand typical forces that act on the bone, without the support of the bone in an unbroken state thereof. However the implant bears at least some of the forces applied to the bone in which implanted.

A particular feature of some embodiments of the invention is that stress is better distributed through the bone, due to better integration of the implant with surrounding bone (e.g., due to inter-digitations), better support of cortical bone (e.g., due to cortical and/or trabecular resting points) and/or composite material and design of implant. In an exemplary embodiment of the invention, the cement support force distribution between the tensile elements, for example, at least 50% of force coupling between tensile elements is due to cement.

A broad aspect of some embodiments of the invention relates to constructing an implant through an opening that is smaller than the implant, where at least some of the load and/or tension bearing elements are non-expanding. Optionally, a ratio of element length to opening diameter is greater than 5:1, 7:1, 10:1 or intermediate values. Optionally or alternatively, at least 3 substantially identical elements are inserted. Optionally, the final implant has a diameter greater by a factor of at least 2, 3, 4, 5 or more than said opening.

In an exemplary embodiment of the invention, an implant comprises a plurality of tensile elements, each with a diameter of less than 20% of the diameter of the final implant and/or the diameter of tensile parts thereof. During construction, the elements are one after the other to lie side by side inside the bone and at least partially define the size and/or mechanical properties of the implant. Optionally, adhesion is provided by injecting a bone cement. Optionally or alternatively, the tensile are designed to interlock with each other, for example including matching recesses and projections. Optionally or alternatively, interconnection between the elements and surrounding matrix is enhanced by the elements being formed with a material that bonds well to the matrix, optionally the same material (e.g., PMMA based rods with a PMMA matrix).

An aspect of some embodiments of the invention relates to in-situ constructing of an implant. In an exemplary embodiment of the invention, the implant is constructed by implanting a bag, inserting a plurality of rods and injecting cement. Optionally, the implant is completed by inserting a further rod. Optionally, the last rod (or another rod) is long enough so that its proximal end lies in a plane of the cortical bone. Optionally, the cement leaks out of the bag to form inter-digitations with surrounding bone. Optionally or alternatively, the cement leaks out of the bag as a distal end point to form an anchoring section.

In an exemplary embodiment of the invention, a rod set is provided, with a plurality of rods and one rod optionally longer than other rods.

In an exemplary embodiment of the invention, a rod is designed to that it slips past other rods, for example, rods including a distal rounded end (optionally designed to not tear the bag) and an inclined and optionally sharp proximal end, to support slippage of the rounded end between two or more previously inserted rods.

An aspect of some embodiments of the invention relates to a composite implant which includes a plurality of materials therein that interact to distribute forces along the implant and hold the implant together. Optionally, tensile elements are not adapted to anchor in bone. Alternatively or additionally, a hardening material, such as adhesive, serves to interconnect the tensile elements and/or transfer forces between them, alternatively or additionally, to separating them. In an exemplary embodiment of the invention, the implant is constructed in-situ. In a bag based implant, a plurality of longitudinal elements of the bag, may each act as a tensile element, independently of a cement holding function of the bag.

An aspect of some embodiments of the invention relates to a method of strengthening a long bone in which an elongate bone strengthening implant(s) is inserted into the bone through an opening made away from the bone ends, for example, the opening not being within 25% of the length of the bone from either end. Alternatively, the implant is inserted from an opening which is closer to one of the bone ends. In an exemplary embodiment of the invention, when a proximal femoral bone is treated, the opening is located at the lateral side of the proximal femoral shaft, for example slightly below the line of the lesser trochanter. In an exemplary embodiment of the invention, the opening location is selected to minimize damage and/or weakening of the bone, at least with respect to certain failure modes of the bone. In an exemplary embodiment of the invention, the implant is inserted along a path drilled from the opening. In an exemplary embodiment of the invention, the path is drilled without a guide wire.

In an exemplary embodiment of the invention, the implant comprises a volume of cement, optionally enclosed in a container such as a bag. Optionally, a container is an elongated bag comprising longitudinally oriented fibers implanted so that the longitudinally oriented fibers are substantially parallel to the longitudinal axis of the bone section being strengthened.

In an exemplary embodiment of the invention, the implant is implanted along an elongate formed channel which is optionally curved to reach into a trochanter of a femur.

An aspect of some embodiments of the invention relates to a kit for implant construction and provision, including a bag holder, a tensile element carrier adapted to insert tensile elements into the bag and a cement injector, all of which are optionally designed to operate via a cannula. Optionally, a bag cutter is provided for cutting or removing the bag from said holder after forming of the implant.

In an exemplary embodiment of the invention, a tool used to drill the channel is controlled using external forces to follow a desired channel. Alternatively or additionally, the tool is guidable (e.g., includes an orientable head) to follow a desired path. Alternatively or additionally, the tool is guided by the cortical layer of the bone. Alternatively or additionally, the tool is guided by a K-wire, previously inserted into bone.

An aspect of some embodiments of the invention relates to a drilling guidewire. In an exemplary embodiment of the invention, the guide-wire has a substantially uniform shaft suitable for delivering tools thereover into or to bone. In an exemplary embodiment of the invention, the guide wire has a handle adapted to fit into a drill in the same manner as a drill bit does.

An aspect of some embodiments of the invention relates to a bone drill including both a forward bone drilling element and a side drilling element. In an exemplary embodiment of the invention, the side drilling element is selectively extendible so that said bone drill can have a substantially uniform diameter along its length when the side element is retracted. Optionally, the side drilling element comprises a bone knife edge that is configured to cut bone when said drill is rotated and retracted. In an alternative embodiment, the side cutting element is replaced by a water jet or jet of other material. Alternatively, the side cutting element is strong enough to cut bone when the drill is rotated at a low speed, such as 1 RPM, 10 RPM, 20 RPM or greater or intermediate speeds. Optionally, for example while rotating, the side cutter is moved in and out along the drills haft using a rail or lumen.

In an exemplary embodiment of the invention, the bone drill is mounted on a shaft strong enough to be used for advancing the bone drill through cortical bone.

In an exemplary embodiment of the invention, the drill head drills when rotated in ether direction. In an exemplary embodiment of the invention, the side element operates in either rotation direction of the shaft and in forward and/or backwards axial movements of the shaft.

In an exemplary embodiment of the invention, the side element extends by the use of a pushing force guided along a lumen or rail from outside the body. Optionally, a threading is provided so a knob can be rotated and push the side element. Optionally, the side element is the extension of a rod that reaches form the knob to inside the body.

An aspect of some embodiments of the invention relates to a drill adapted to create a curved path in a bone. In an exemplary embodiment of the invention, the drill includes a head coupled to a first, curved, elongate inner element contained within a second, outer, tube which is stiffer than the inner element. In use, the outer tube is advanced over the inner element when a part with a first curvature (e.g., including a straight line) is desired. When the outer tube is retracted, the curvature of the inner element takes over and defines a curved path. Optionally, the head comprises a burr on a wire and the inner element is a tube enclosing the wire. Optionally, the size of opening created by the head depends on the distance between the burr and the inner element.

In another embodiment of the invention, a curved tube, for example a tube having a “banana” shape, is used for drilling a channel and/or implant insertion. In an exemplary embodiment of the invention, the curved tube is made of nickel-titanium (Nitinol). In another exemplary embodiment of the invention a burr, connected to a rotator flexible shaft, is incorporated at the head of said tube. In an exemplary embodiment of the invention, the curved tube is guided, for example by a K-wire, optionally a curved K-wire.

In other embodiments of the invention, a straight channel is made for implant insertion.

In an exemplary embodiment of the invention, said drill incorporates water-jetting drilling and/or reaming capabilities, e.g., drilling and/or reaming bone tissue by high speed water jets with or without abrasive particles.

A broad aspect of some embodiments of the invention relates to injecting exothermic-setting cement while minimizing damage to healthy bone.

In an exemplary embodiment of the invention, the cement is formulated to produce less heat, even if this reduces the strength of the set cement. For example, the ratio of monomer to powder in PMMA may be skewed so that there is less monomer than typical and/or larger than usual beads may be used. In an exemplary embodiment of the invention, the bending strength of the set cement is allowed to be similar to that of bone or for example 20% less, 40% less or even 60% less, or less than 120% or intermediate values. It is noted that the cement optionally serves to replace trabecular bone. Optionally the completed implant has an elasticity modulus of between 5-80 Gpa.

Optionally, reduction in strength is compensated for, at least in part, by tensile implants.

In an exemplary embodiment of the invention, the cement, of any type, is cooled during setting using an elongate cooling element that remains in the cement as it sets, optionally serving as a tensile element.

There is provided in accordance with an exemplary embodiment of the invention, an elongate bone implant, comprising:

in-situ hardened material; and

a plurality of longitudinal tensile elements in contact with said hardened material, which tensile elements have a greater tensile strength than said hardened material,

wherein at least one of said tensile elements is not adapted for anchoring to bone.

In an exemplary embodiment of the invention, said at least two of said tensile elements are interlocked only by said hardened material. Optionally or alternatively, forces are carried between said at least two of said tensile elements only by said hardened material. Optionally or alternatively, said at least two of said tensile elements are spaced apart by said hardened material. Optionally or alternatively, said plurality of tensile elements and said hardened material act together as a composite material whose mechanical properties are determined by the combination of the hardened material and the tensile elements.

In an exemplary embodiment of the invention, said implant has a length/diameter ratio of greater than 1:3.

In an exemplary embodiment of the invention, said implant is configured to have an elasticity modulus greater than 5 Gpa. Optionally or alternatively, said implant is configured to have elasticity modulus less than 80 Gpa.

In an exemplary embodiment of the invention, said hardened material comprises bone cement.

In an exemplary embodiment of the invention, said hardened material comprises adhesive material.

In an exemplary embodiment of the invention, said longitudinal tensile element comprises at least one bag surrounding at least a portion of said hardened material. Optionally, said longitudinal tensile element comprises nested bags. Optionally or alternatively, said bag has an elongation of less than 10%. Optionally or alternatively, said bag has a plurality of longitudinal fibers. Optionally or alternatively, said bag has a leading edge configured to hold a rod. Optionally or alternatively, said bag has a plurality of general pores formed therein supporting a seeping of said hardened material prior to hardening thereof. Optionally, said seeping forms inter-digitations. Optionally or alternatively, said bag has a plurality of specific pores formed therein and having an average cross-section of at least 50% greater than that of said general pores. Optionally or alternatively, said bag has a first seepage area and a second seepage area, said first area having an effective aperture area greater by at least 50% than that of said second seepage area, said effective aperture area calculated by adding up aperture areas and dividing by the seepage area.

In an exemplary embodiment of the invention, said implant does not include a bag enclosing at least 50% of said hardened material.

In an exemplary embodiment of the invention, there is provided a set of a plurality of spaced apart and substantially axially parallel implants as described herein.

In an exemplary embodiment of the invention, said plurality of tensile elements comprises at least one elongate element of a diameter smaller than 50% than of an average diameter of said implant, as measured along a long axis of said implant. Optionally, said elongate element has a diameter of less than 4 mm. Optionally or alternatively, said plurality of tensile element comprises at least one elongate element of a diameter smaller than 1 mm. Optionally or alternatively, said plurality of tensile elements comprises at least one elongate element of a diameter smaller than 0.1 mm. Optionally or alternatively, said plurality of tensile elements comprises at least one elongate element of a diameter smaller than 0.01 mm. Optionally or alternatively, at least one of said tensile element is flexible. Optionally or alternatively, at least one of said tensile element is rigid and bend-resistant. Optionally or alternatively, a first one of said tensile elements has a shaped proximal end and a second one of said elements has a shaped distal end, said shaped ends configured for sliding past each other. Optionally or alternatively, the implant comprises a plurality of elements filling at least 30% of said implant volume. Optionally or alternatively, the implant comprises a plurality of elements filling at most 80% of said implant volume. Optionally or alternatively, the implant comprises at least one rigid bend resisting element having a length of substantially an entire length of said implant in bone. Optionally or alternatively, at least one of said at least one elements includes a radio-opaque portion. Optionally or alternatively, at least one of said at least one elements has a geometry selected for encouraging engagement of said hardened material during hardening thereof.

In an exemplary embodiment of the invention, said implant is substantially straight.

In an exemplary embodiment of the invention, said implant is curved to have a diameter of a smallest object of rotation thereof greater than 200% of an average diameter by length of said implant.

In an exemplary embodiment of the invention, the implant is constituted to have a density within 50% of the density of trabecular bone.

There is provided in accordance with an exemplary embodiment of the invention, an implant kit comprising:

(a) an elongate sack;

(b) a plurality of tension rods having a length and diameter suitable for fitting of at least 3 rods in said sack; and

(c) filler material precursors of an amount suitable to fill said sack with said rods therein. Optionally, said sack has a distal end including a reinforcement adapted to receive a distal end of at least one of said rods. Optionally or alternatively, at least one of said rods is longer than others of said rods by at least 5% of an average rod length. Optionally or alternatively, at least one of said rods is longer than others of said rods by at least 5 mm. Optionally or alternatively, said sack is perforated to support a seepage of said filler material over at least a portion of said bag. Optionally or alternatively, said sack is additionally perforated at a distal end thereof to support the formation of a bulb of filler material thereat. Optionally or alternatively, each of said rods has a diameter smaller than 4 mm. Optionally or alternatively, a first one of said rods has a shaped proximal end and a second one of said rods has a shaped distal end, said shaped ends configured for sliding past each other.

There is provided in accordance with an exemplary embodiment of the invention, a method of preventive surgery, comprising:

(a) identifying a long bone in need of strengthening; and

(b) implanting a strengthening implant in said bone through an aperture formed in the bone. Optionally, said bone is a hip. Optionally or alternatively, said bone is not indicated as fractured by said identifying. Optionally or alternatively, implanting comprises binding at least two spaced apart reinforcing elements with a binding material. Optionally or alternatively, said strengthening implant comprising a tension-resistant element. Optionally or alternatively, said strengthening implant comprising a bend-resistant element. Optionally or alternatively, the method comprises selecting a personalized dimension for said implant for said bone. Optionally or alternatively, said implant is configured to rest against a cortex of said bone at one end and in a middle section thereof. Optionally or alternatively, identifying comprises providing a patient with a problem in one limb and treating both that limb and an opposing limb, by implantation of implants therein.

There is provided in accordance with an exemplary embodiment of the invention, a method of preventive surgery, comprising:

(a) identifying a long bone in need of strengthening; and

(b) building, in situ, a strengthening implant formed of a hardening material and at least one reinforcing element, which reinforcing element is not adapted to anchor in bone.

In an exemplary embodiment of the invention, building comprises:

(c) forming a void in said bone; and

(d) constructing said implant in said void.

Optionally, forming a void comprises:

(e) forming a channel; and

(f) widening said channel.

Optionally, widening said channel comprises cutting said channel using a cutting element. Optionally or alternatively, forming a channel comprises forming a curved channel.

In an exemplary embodiment of the invention, forming a void comprises forming a plurality of voids. Optionally or alternatively, forming a void comprises forming a void having a distal end not contacting and within about 5 mm of a cortical bone. Optionally or alternatively, constructing said implant comprises inserting at least one tensile element into said void and filling said void using cement. Optionally, inserting at least one tensile element comprises inserting a bag into which said cement is provided. Optionally or alternatively, inserting at least one tensile element comprises inserting a second bag into said bag. Optionally, filling said void comprises eluting at least part of said cement out of said bag to form inter-digitations. Optionally or alternatively, filling said void comprises eluting at least part of said cement out of said bag to form at least one bulbous anchor section. Optionally or alternatively, inserting at least one tensile element, comprises inserting a plurality of tensile elements into said bag. Optionally or alternatively, inserting at least one tensile element, comprises inserting a tensile element having at least one end in a cortex. Optionally or alternatively, inserting at least one tensile element, comprises inserting said bag using a tensile element.

In an exemplary embodiment of the invention, inserting at least one tensile element comprises inserting a bag into which said cement is provided.

In an exemplary embodiment of the invention, said method is practiced by forming a hole having a maximal diameter of less than 5 mm. Optionally or alternatively, said method is practiced by forming a hole having a maximal diameter of less than 3 mm.

There is provided in accordance with an exemplary embodiment of the invention, a kit for constructing an implant in situ, comprising:

(a) a void former adapted to form an elongate void in bone;

(b) a carrier configured to provide at least one elongated structural element into said void; and

(c) a filler material injector configured to provide filler material into said void.

Optionally, said at least one elongated element is a tension-resistant element. Optionally or alternatively, said at least one elongated element is a bend-resistant element. Optionally or alternatively, said carrier is configured to push a longitudinal element into said void. Optionally, the kit comprises a sack carrier adapted to insert a sack into said void. Optionally, said tensile element carrier is configured to match said bag carrier and said bag in length so that it pushes said element to an end of said bag.

In an exemplary embodiment of the invention, said tensile element carrier is configured to release a tensile element into said void as said tensile element carrier is retracted.

In an exemplary embodiment of the invention, said void former is bendible.

In an exemplary embodiment of the invention, said void former comprises a narrow void former and a void widener. Optionally, said void widener comprises a cutting element.

In an exemplary embodiment of the invention, the kit comprises a cannula adapted for bone access and engaging a bone cortex and sized to pass the intrabody portions of said void former and said tensile element carrier.

In an exemplary embodiment of the invention, the kit comprises a bag and a plurality of tensile elements in the form of rods.

There is provided in accordance with an exemplary embodiment of the invention, a device for disposing longitudinal rods within a cavity in bone, comprising:

(a) a handle;

(b) a shaft adapted to be inserted into a bone cavity through a cannula; and

(c) a rod holder formed at least at a distal end of said shaft and adapted to hold a rod.

Optionally, said rod holder has an outer diameter along most of said shaft which is less than 20% greater than a diameter of a rod it is adapted to hold. Optionally or alternatively, said device is adapted to dispose rods with a diameter of less than 5 mm.

In an exemplary embodiment of the invention, the carrier comprises a pusher adapted to release said rod from said rod holder by pushing against a proximal end of said rod.

In an exemplary embodiment of the invention, the carrier comprises a magazine holding at least three rods.

There is provided in accordance with an exemplary embodiment of the invention, a bone drill comprising:

(a) a shaft with a substantially uniform diameter and adapted for cannulated access to bone and including a distal end configured for attachment to a motorized drill handle;

(b) a bone drilling head at a distal tip of the shaft and adapted for drilling into both cortical bone and trabecular bone,

wherein said drill has a maximal diameter within 10% of an average diameter of said drill, by length.

Optionally, said shaft is rigid. Optionally or alternatively, the drill comprises:

(c) at least a side extending element configured to selectively extend in a direction perpendicular to said shaft and stiff enough when extended to cut trabecular bone at a rotation speed below 20 RPM. Optionally, said side-extending element comprises a wire. Optionally or alternatively, said side-extending element comprises an edged cutting element. Optionally or alternatively, said side-extending element is extended along a linear passage in said shaft. Optionally, said side-extending element is extended by a pushing force applied from outside a body. Optionally or alternatively, the drill comprises a shield preventing bone chips from entering in to said passage.

There is provided in accordance with an exemplary embodiment of the invention, a long bone implant comprising:

(a) a plurality of stiff rods; and

(b) a binding material hardened in-situ,

wherein at least one of said rods has a length of more than 50% of a length of said implant

Optionally, said implant has inner shear resistance that is substantially equivalent to that of cancellous bone. Optionally or alternatively, at least two of said rods are spaced apart by said binding material. Optionally or alternatively, said implant further comprising a meshed element through which said binding material seeps.

There is provided in accordance with an exemplary embodiment of the invention, a method of constructing an implant comprising:

(a) forming an opening in cortical bone, which opening is less in diameter than a 30% of a diameter of said bone;

(b) inserting at least three non-expanding elements through said opening into said bone;

(c) completing said implant to have a diameter at least 3 times said opening diameter.

In an exemplary embodiment of the invention, said non-expanding elements are elongate elements with a length to diameter ratio of greater than 3 to 1.

There is provided in accordance with an exemplary embodiment of the invention a method of strengthening a bone, comprising providing at least one implant which lays mostly inside a cortical portion of said bone.

There is provided in accordance with an exemplary embodiment of the invention a method of strengthening a bone, comprising providing at least one implant into said bone, said implant resting at one end thereof against at least one void in cortical bone and resting at a section within a middle 50% of length of said implant inside bone, against an inside surface of cortical bone. Optionally, the method includes setting at least one resting place of said implant against bone by eluting cement out of said implant.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

### BRIEF DESCRIPTION OF THE DRAWINGS

In the drawings which follow, identical structures, elements or parts that appear in more than one drawing are generally labeled with the same numeral in all the drawings in which they appear. Dimensions of components and features shown in the drawings are chosen for convenience and clarity of presentation and are not necessarily shown to scale.

FIG. 1 is a flowchart of a method of strengthening a bone to prevent fractures, in accordance with an exemplary embodiment of the invention;

FIGS. 2A-2J is a series of figures showing a process of forming a bore in a hip bone and various configurations thereof, in accordance with an exemplary embodiment of the invention;

FIGS. 2K-2M is a series of figures showing examples of implant locations in accordance with an exemplary embodiment of the invention;

FIGS. 3A and 3B illustrate a distal end of a drilling device used in FIG. 2, in a straight and in a curved state, in accordance with an exemplary embodiment of the invention;

FIGS. 4A-4D depict axial cross sections of bones treated in accordance with the teachings of the present invention so that a cross section of embodiments of implants of the present invention are apparent;

FIGS. 5A-5B depict longitudinal cross sections of bones treated in accordance with the teachings of the present invention so that a cross section of embodiments of implants of the present invention are apparent;

FIGS. 6A-6B depict an axial cross section and a longitudinal cross section of a bone treated in accordance with the teachings of the present invention having tensile elements implanted inside the bone;

FIG. 7A illustrates a cross section view of a longitudinal implant, in accordance with an exemplary embodiment of the invention;

FIG. 7B illustrates a cross section view of a bone cavity occupied by a composite implant comprising a plurality of longitudinal elements and binding material, in accordance with an exemplary embodiment of the invention;

FIGS. 8A-8D illustrate cross sections of several drilling heads which incorporate water jet techniques, in accordance with an exemplary embodiment of the invention;

FIGS. 9A-9B illustrate cross sections of an inflatable drilling head, in accordance with an exemplary embodiment of the invention;

FIG. 10A is a flowchart of a method of composite bone implant construction and implantation, in accordance with an exemplary embodiment of the invention;

FIG. 10B is a cross-sectional view of a composite implant in a bone, in accordance with an exemplary embodiment of the invention;

FIGS. 10C-10I illustrate acts in the method of FIG. 10A, using the tools of FIGS. 11-19, in accordance with an exemplary embodiment of the invention;

FIGS. 11-19 illustrate components of a bone implant kit usable for the method of FIG. 10A, in accordance with an exemplary embodiment of the invention;

FIG. 11 illustrates a bone access cannula, in accordance with an exemplary embodiment of the invention;

FIGS. 12A-12E illustrate a bone drill, in accordance with an exemplary embodiment of the invention;

FIG. 13 illustrates the bone drill of FIGS. 12A-12E mounted in the bone access cannula of FIG. 11, in accordance with an exemplary embodiment of the invention;

FIG. 14 illustrates a stylet, in accordance with an exemplary embodiment of the invention;

FIG. 15 illustrates a cement delivery cannula with the stylet of FIG. 14 mounted therein, in accordance with an exemplary embodiment of the invention;

FIGS. 16A-16C illustrate a bag holder in accordance with an exemplary embodiment of the invention;

FIG. 17 illustrates the stylet of FIG. 14, mounted in the cement delivery cannula of FIG. 15, mounted in the bag holder of FIGS. 16A-16C, all mounted in the bone access cannula of FIG. 11, in accordance with an exemplary embodiment of the invention;

FIGS. 18A-18B illustrate a rod carrier, in accordance with an exemplary embodiment of the invention;

FIG. 19 illustrates the rod carrier of FIGS. 18A-18B, mounted in the bag holder of FIGS. 16A-16C, all mounted in the bone access cannula of FIG. 11, in accordance with an exemplary embodiment of the invention;

FIGS. 20A and 20B illustrate an alternative side drill extension, in accordance with an exemplary embodiment of the invention;

FIG. 20C-20E illustrate another alternative side drill extension, in accordance with an exemplary embodiment of the invention;

FIG. 21A-21B illustrate multi-rod pushers, in accordance with exemplary embodiments of the invention;

FIG. 22 illustrates a multi-tool system, in accordance with an exemplary embodiment of the invention;

FIGS. 23A and 23B illustrate tensile rods, in accordance with exemplary embodiments of the invention;

FIG. 24A-24D illustrate bag attachment methods in accordance with an exemplary embodiment of the invention;

FIG. 25 illustrates a sleeve cutting tool, in accordance with an exemplary embodiment of the invention; and

FIG. 26 illustrates a weave of a bag including a pore, in accordance with an exemplary embodiment of the invention.

### DETAILED DESCRIPTION OF EXEMPLARY EMBODIMENTS

### Overview

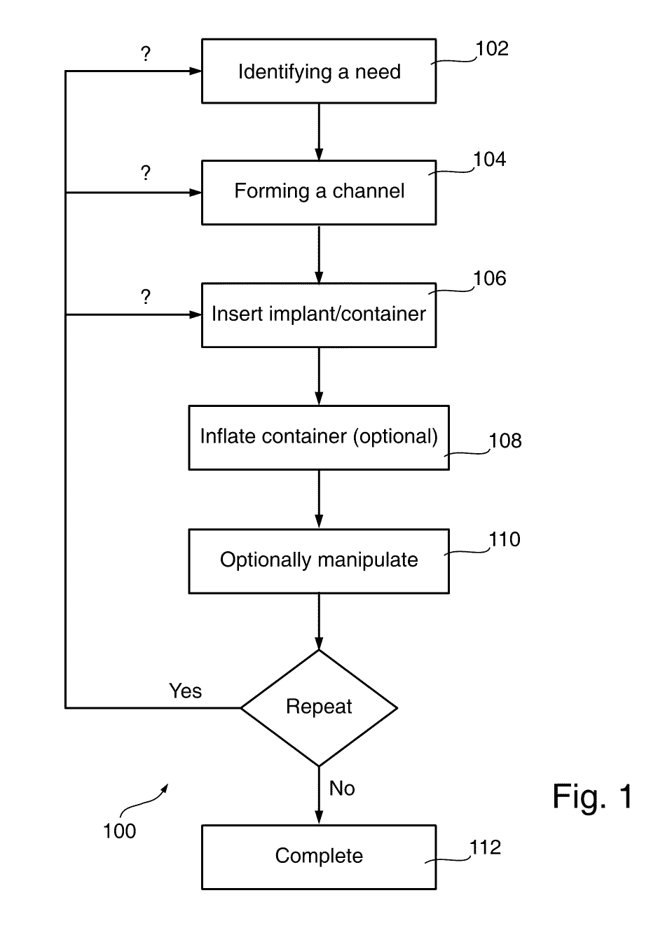

FIG. 1 is a flowchart 100 of a method of strengthening a bone, for example to prevent fractures, in accordance with an exemplary embodiment of the invention. In brief, the method includes identifying a need (102), forming a channel in the bone (104), inserting an implant and/or a container (106), optional inflating the container using cement (108), optional repeating or otherwise manipulating the container (110) and completing the procedure (112).

### Exemplary Prophylactic Treatment

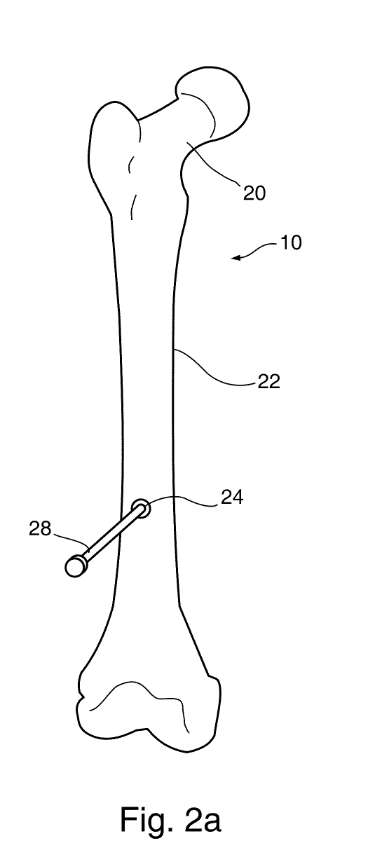

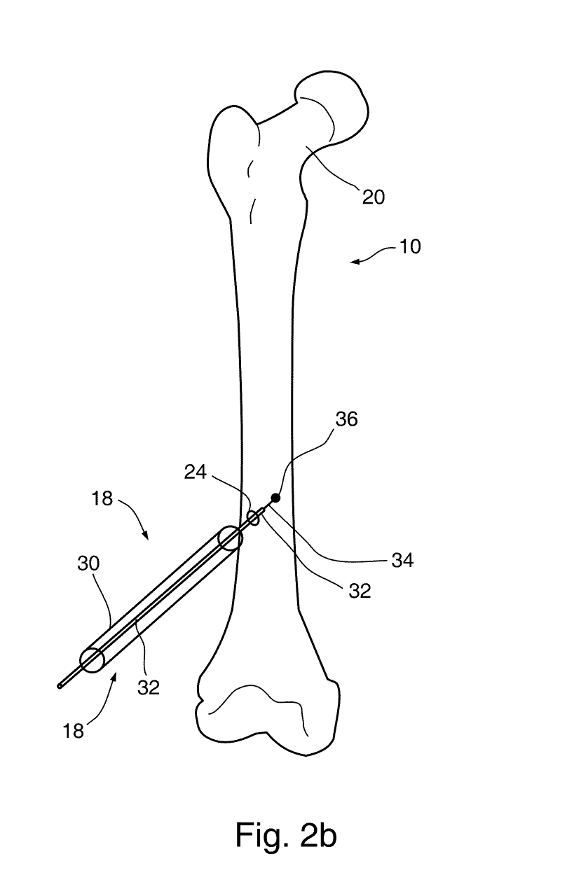

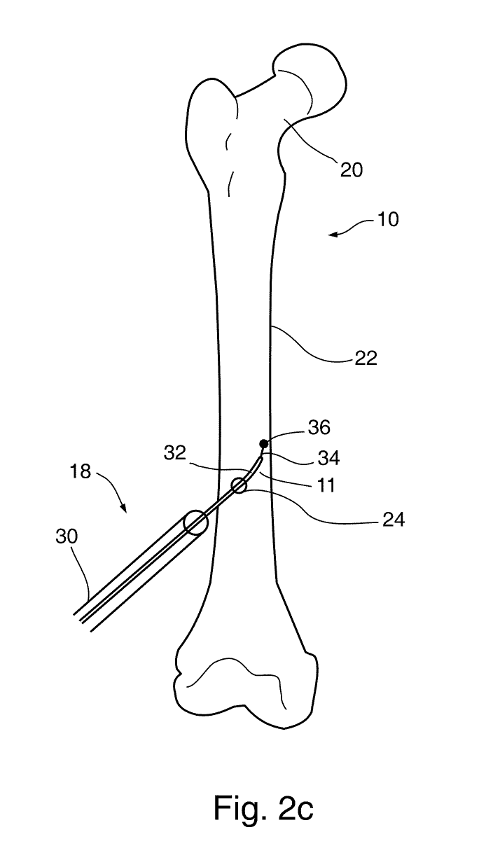

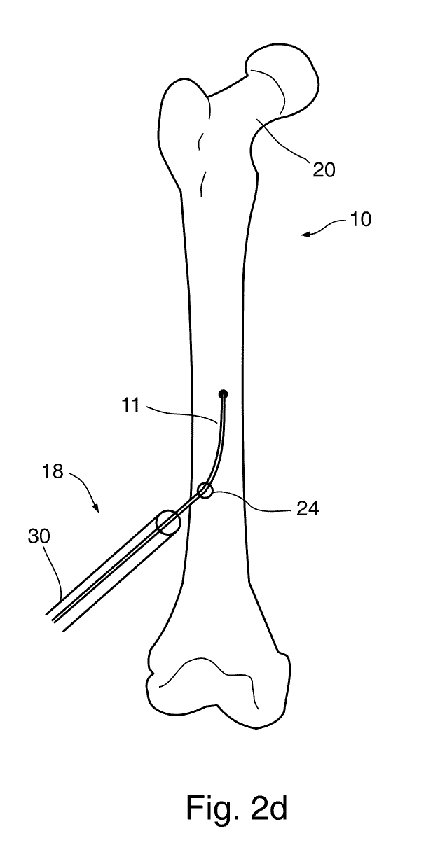

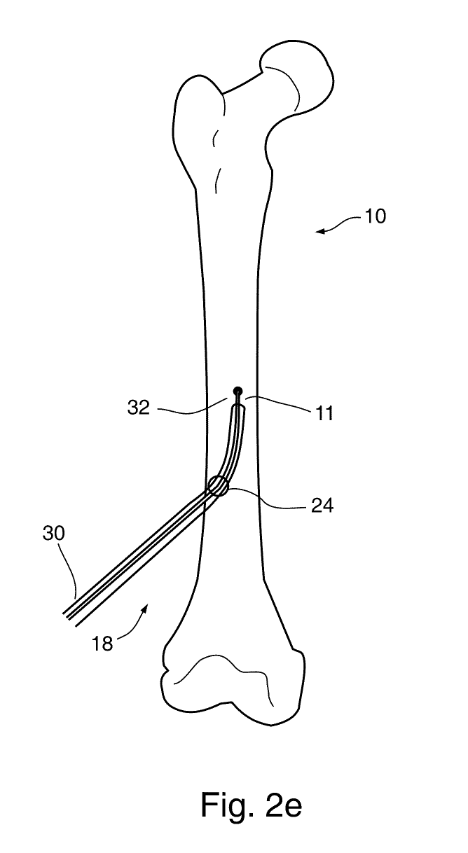

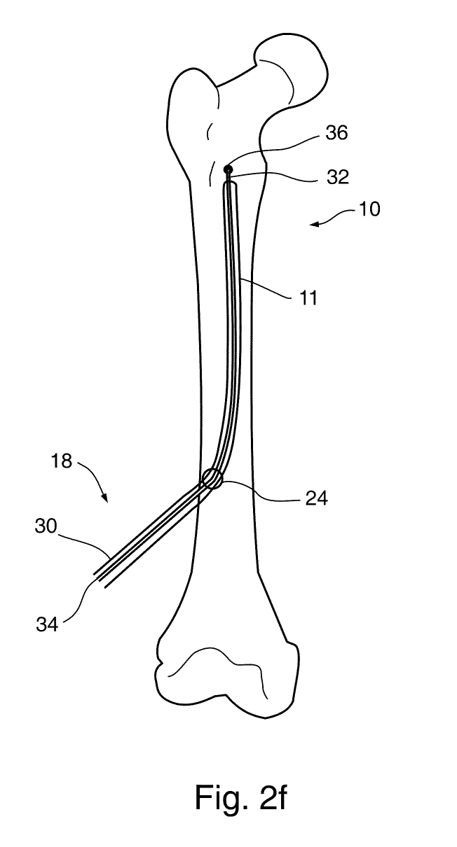

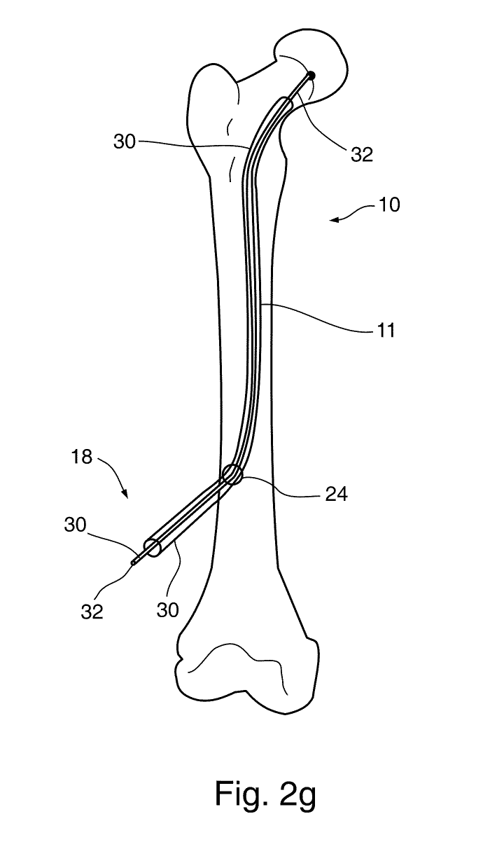

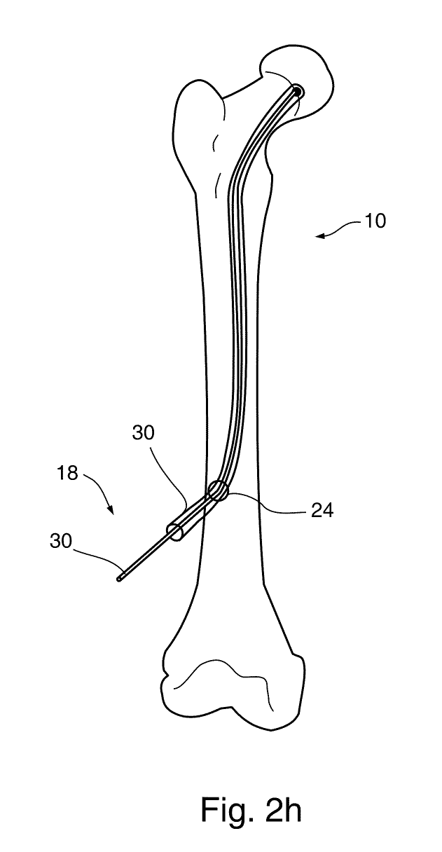

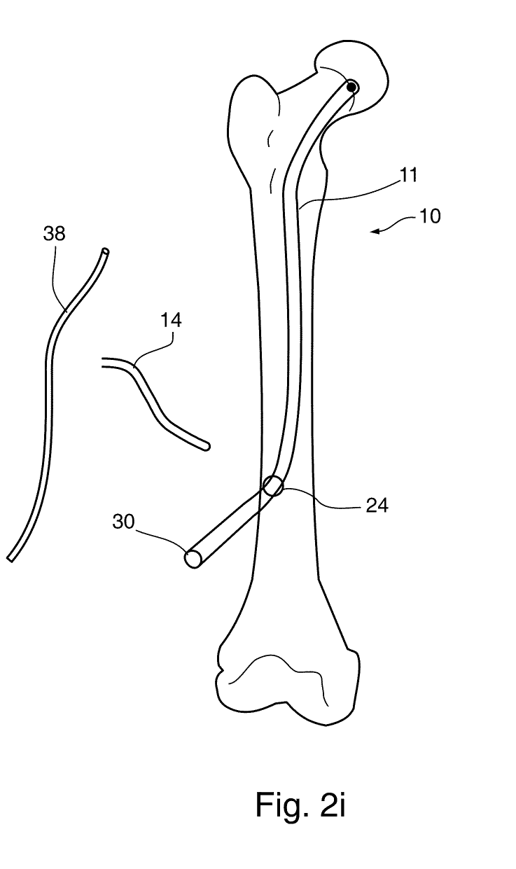

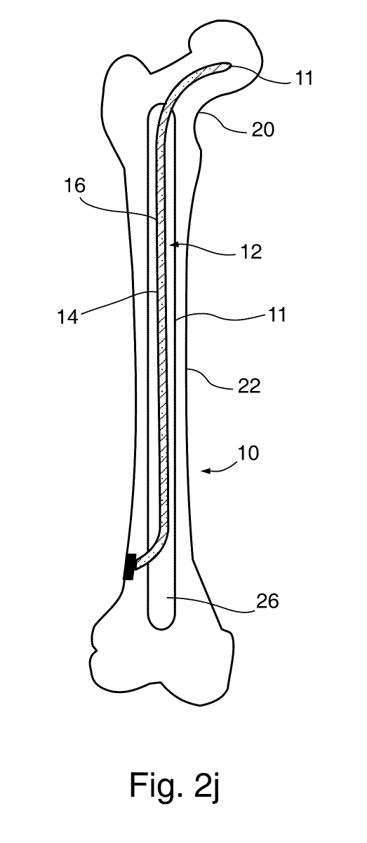

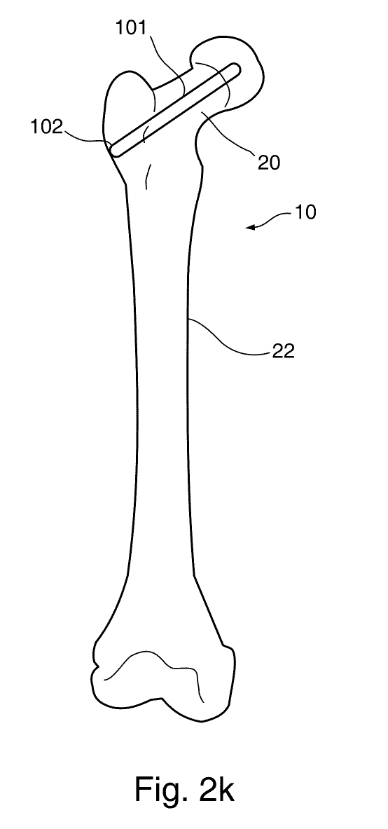

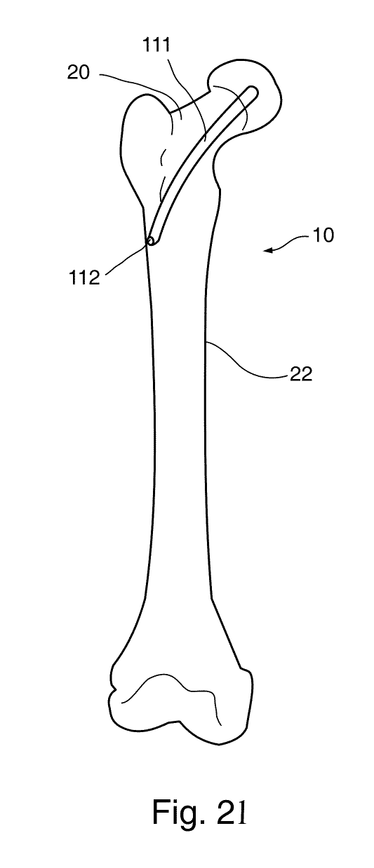

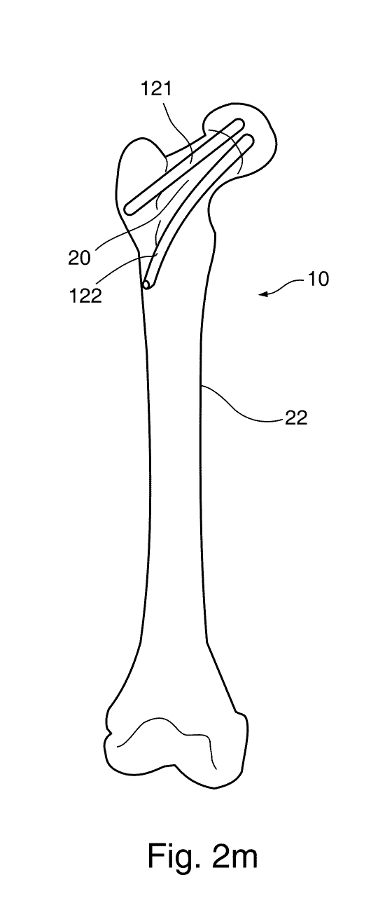

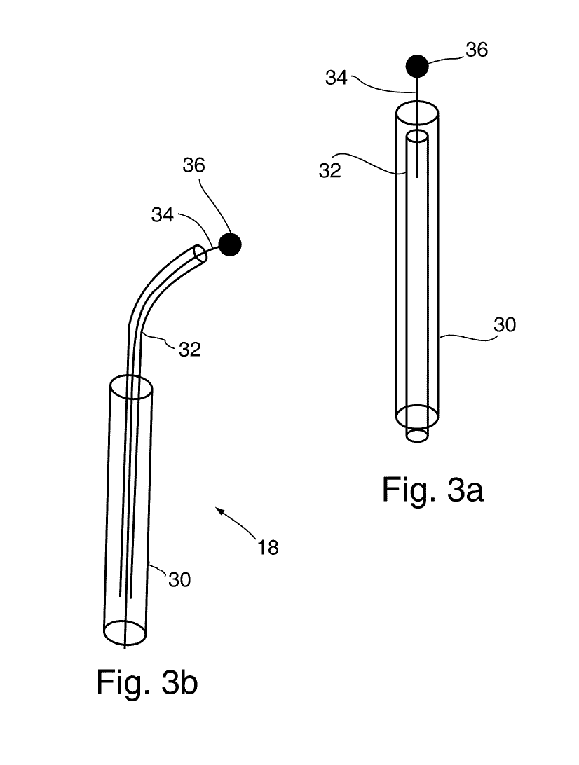

The method schematically depicted in FIG. 1 is explained for a specific prophylactic treatment of a non-fractured femur 10 as depicted in FIGS. 2A-2M. However, the treatment is not limited for preventive purposes, and may also be used for the treatment of broken bones. In these figures is described the implantation of an implant 12 of the some embodiments of the present invention. FIG. 2I depicts the implant fully assembled and deployed in a longitudinal channel 11 running through femur 10. This channel is optionally formed using a drilling device 18 depicted in FIGS. 3A and 3B. Implant 12 optionally comprises an elongated fiber bag 14 as a tensile element that is a container filled with a cement 16 such as PMMA

### Identify Need

In 102, a need is identified for treating a bone. Typical identifying of a need includes, for example, one or more of determining a sufficiently high likelihood of a fracture of a specific long bone, for example due to a history of a fracture in the bone, history of fracture of a same bone on the other side of the body, identification of microfractures and cracks in a bone and osteoporosis. In FIG. 2, femur 10 is not fractured but the opposing femur of the same patient was previously fractured at the femoral neck 20.

### Form Channel

At 104, a longitudinal channel 11 is formed substantially in parallel to shaft 22 of femur 10 extending up into femoral neck 20. Alternatively, a curved or straight channel is formed, where its opening is located at a higher point in the femur shaft (i.e., closer to the bone proximal end).

An insertion hole 24 (between about 2 mm and about 15 mm in diameter) is drilled through the compact tissue constituting the wall of shaft 22 into the medullary cavity 26 of femur 10, for example, using a standard rigid drill 28 in accordance with methods known in the art. The location where insertion hole 24 is made is optionally distal from femoral neck 20 and through compact tissue. This may reduce pain, complications and/or reduce further weakening of regions in proximity to femoral neck 20.

Channels in the soft tissue surrounding the bone is optionally made using minimally invasive methods (e.g., small hole of several mm), such as using drill 28, or using an open cut.

As noted above, in an embodiment of the invention depicted in FIG. 2, a suitable longitudinal channel 11 is formed in femur 10 with the help of a drilling device 18 depicted in FIGS. 3A and 3B. In some embodiments, the channel is along the trochanter.

A first exemplary drilling device 18 is substantially analogous to a catheter type device. In FIGS. 3A and 3B is depicted only the distal end of drilling device 18 which comprises an outer guide tube 30 through which bore runs inner drill guide tube 32 through which bore runs a rotation wire 34 tipped with an excavation component, for example, a standard bone drilling burr 36. In an exemplary embodiment of the invention, inner tube 32 is relatively flexible (compared to outer guide tube 30, and in embodiments elastic and is configured to curve at the tip to one side. Rotation wire 34 is functionally associated with a rotator such as a high speed electrical motor known in the art of surgical drills. In an alternative exemplary embodiment of the invention (not shown), tube 30 has a tilting distal end which can be manipulated from outside the body (via tube 30 proximal end), such that the operator can choose between a first straight position and a second tilted position, or vise versa. Optionally, the preferred tilt angle can also be determined and carried out. Optionally, pull wires are used to effect such manipulations

In embodiments, guidance of a drilling device is performed in other manners, for example using magnetic guidance to magnetically manipulate the drilling tip (which may be selectively made of a ferromagnetic material or act as an electromagnet).

In embodiments, a drilling device such as drilling device 18 is configured to aspirate material excavated by an excavation component, for example by being functionally associating with a pump or another vacuum source.

In embodiments, an excavation component constitutes, in addition to or instead of a burr such as burr 36, a different excavation component. Typical such excavation components include but are not limited to hard piercing members (optionally configured to tunnel through the tissue in a medullary cavity and/or trabecular bone but not cortical bone), ultrasonic transducers, electro ablators, drills, reamers, fluid jets, morsolaters, electrical ablators, vibrating needles and/or laser drilling devices. In embodiments, an excavation element, such as a burr are configured to have a variable configuration (e.g. by controlling the axial distance of burr 36 from inner tube 32) to change the diameter of a channel made with the excavation element. Typically, the diameter of a longitudinal channel 11 excavated in a femur is not less than 2 mm, not less than 3 mm, not less than 4 mm, not less than 5 mm, not less than 6 mm and even not less than 8 mm or intermediate values.

In an exemplary embodiment of the invention, for forming longitudinal channel 11, burr 36 of drilling device 18 is inserted through insertion hole 24 into medullary cavity 26 of femur 10 when inner tube 32 is substantially entirely within outer tube 30 so that burr 36 is substantially near the end of outer tube 30, as depicted in FIG. 3A. Inner tube 32 is pushed outwards from outer tube 30. No longer constrained by the relatively rigid outer tube 30, inner tube 32 curves upwards inside medullary cavity 26 towards femoral neck 20, substantially as depicted in FIG. 3B.

The rotator (e.g., motor or manual handle) functionally associated with rotation wire 34 is optionally activated so as to rotate rotation wire 34 and thus burr 36. As burr 36 rotates, inner tube 32 is pushed outwards from outer tube 30 to advance upwards inside against the inner wall of compact tissue, defining medullary cavity 26 and excavating tissue so as to define longitudinal channel 11.

Optionally, outer tube 30 and/or inner tube 32 and/or rotation wire 34 are coupled on their proximal end to an electrical or a mechanical drill, which optionally provides rotation and/or hammer impact (not shown). Optionally, wire 34 is not rotated but rather pulled and pushed with a desired force and/or frequency.

In an exemplary embodiment of the invention, when a sufficient length of longitudinal cavity has been excavated (e.g., as determined by an operator, optionally using x-ray imaging), for example where further excavation may lead to an improperly shaped longitudinal channel, outer tube 30 is advanced inwards so as to support and straighten inner tube 32, see FIG. 2E. In embodiments, inner tube 32 functions as a guide for outer tube 30. In embodiments, during extension of inner tube 32 by a small amount (e.g., 1-2 cm) from outer tube 30, inner tube 32 is straight and can thus be used to excavate a straight part of longitudinal channel 11 while a greater extension (e.g., greater than 3 cm) leads to substantial curvature of inner tube 32.

When longitudinal channel 11 has been excavated to near femoral neck 20 (e.g., near the intertrochanteric line), inner tube 32 is pushed distally out from outer tube 30 so as to curve into femoral neck 20, preferably close to the axis of the femoral neck, see FIG. 2F.

Excavation of tissue in femoral neck 20 is continued as described above until longitudinal channel 11 is sufficiently long. In the embodiment depicted in FIG. 2G, longitudinal channel 11 extends from insertion hole 24 inside shaft 22 until into femoral neck 20.

### Insert Implant

At 106, tensile elements are inserted into longitudinal channel 11, in FIG. 2 a container which is substantially a bag 14. In a typical embodiments, a bag 14 is of knit 25 Dtex Dyneema® (vide infra) with holes of about 0.04 mm² and a maximal inflated diameter 20% greater than that of longitudinal channel 11. While outer tube 30 of drilling device 18 is substantially maintained in place, inner tube 32 is withdrawn together with rotation wire 34 and burr 36, see FIG. 2H. Elongated bag 14 (optionally knotted at the top) is placed over the blunted tip of a flexible inflation and guide tube 38 and pushed upwards to the distal end of longitudinal channel 11 with the help of inflation and guide tube 38.

### Fill Implant

At 108, bag 14 is filled with cement. When elongated bag 14 contacts the end of longitudinal channel 11, cement such as PMMA is injected slowly through inflation and guide tube 38. Stepwise or simultaneously, a portion of cement 16 is injected and inflation and guide tube 38 is withdrawn so that elongated bag 14 fills out and compresses against the tissue defining the walls of longitudinal channel 11. In embodiments, the mass of partially set cement maintains bag 14 in place as inflation and guide tube 38 is withdrawn. Alternatively, a tensile element such as bag 14 is maintained in place with the help of an anchoring component, for example, as described below. In embodiments where setting of cement 16 may release heat, the rate of cement injection is optionally slow enough so as not to cause substantial damage or discomfort to the subject being treated. In some embodiments, an inflation and guide tube 38 is configured to assist in removing heat generated by setting cement, for example by including a channel for the transport of cooling fluid. When sufficient cement 16 has been injected into bag 14, inflation and guide tube 38 is withdrawn from femur 10 out through insertion hole 24.

In embodiments, cement 16 is optionally injected into bag 14 at a pressure sufficient to compact and/or inter-digitate surrounding trabecular bone. In embodiments, cement 16 is optionally injected at such a pressure into bag 14, and the walls of bag 14 are such that cement leaks through portions of the walls of bag 14 so as to achieve interdigitation with bone tissue.

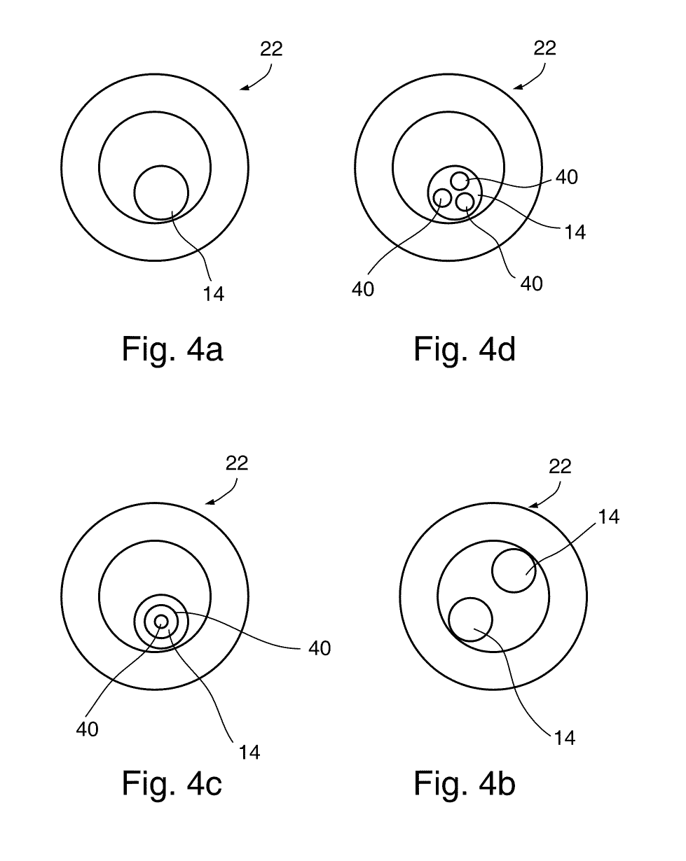

In typical femoral embodiments, an inflated diameter of a container such as a bag is between about 10 mm and 20 mm, for example 15 mm. It is important to note that although in FIGS. 4A-4D bag 14 fills only a small portion of the cross section of the medullary cavity of femur 10, in some embodiments a bag 14 (or several bags) fills a majority of a the medullary cavity, or the medullary cavity in its entirety.

### Repeat and Manipulate

In some embodiments, the steps above are repeated, for example to insert an additional bag 14 in a different femur or in the same femur as depicted in FIG. 4B.

In an exemplary embodiment of the invention, the implant is manipulated, for example, by insertion of tensile elements thereto, for example, as described below.

### Complete Procedure

At 112, the procedure is completed. In an exemplary embodiment of the invention, the proximal end of bag 14 trimmed (optionally as described below), remnants pushed into insertion hole 24, and/or insertion hole 24 blocked with standard bone filling paste such manufactured by Exactech, Inc (Gainseville, Fla., USA).

### Tensile Elements

In some embodiment, such as discussed above, the tensile elements of the implant are exclusively the filaments that constitute bag 14, as depicted in cross section in axial FIG. 4A.

In some embodiments, additional tensile elements are introduced, for example, contained within bag 14. In some embodiments, the added tensile elements are threads, rods and/or additional bags.

### Multiple Bag Layers

In some embodiments, a bag 14 is a multilayer bag, including multiple layers of material defining the walls of bag 14.

In some embodiments, the additional tensile elements are one or more additional bags 40 similar to bag 14, but optionally of smaller maximal diameter and optionally with larger perforations through the walls of the bag. Subsequent to insertion of bag 14 and injection (in some embodiments before and in some embodiments after) of a portion of cement, inflation and guide tube 38 is withdrawn and used to insert additional bags 40 inside bag 14. In some embodiments additional (an additional 1, 2, 3, 4 or even more) bags 40 are inserted coaxially to bag 14, see FIG. 4C. In some embodiments additional bags 40 are inserted collinear but not coaxial to bag 14, see FIG. 4D. As noted, in FIGS. 4C and 4D bag 14 can fill only a small portion of the cross section of the medullary cavity of femur 10, in embodiments a bag 14 fills a majority of a the medullary cavity, or the medullary cavity in its entirety. In some embodiments the bags are inserted side by side inside bag 14. In some embodiments, a strip or other form is used instead of a bag, with a distal end of the strip being adapted for being pushed by a stylet, for example, including a cup or a loop.

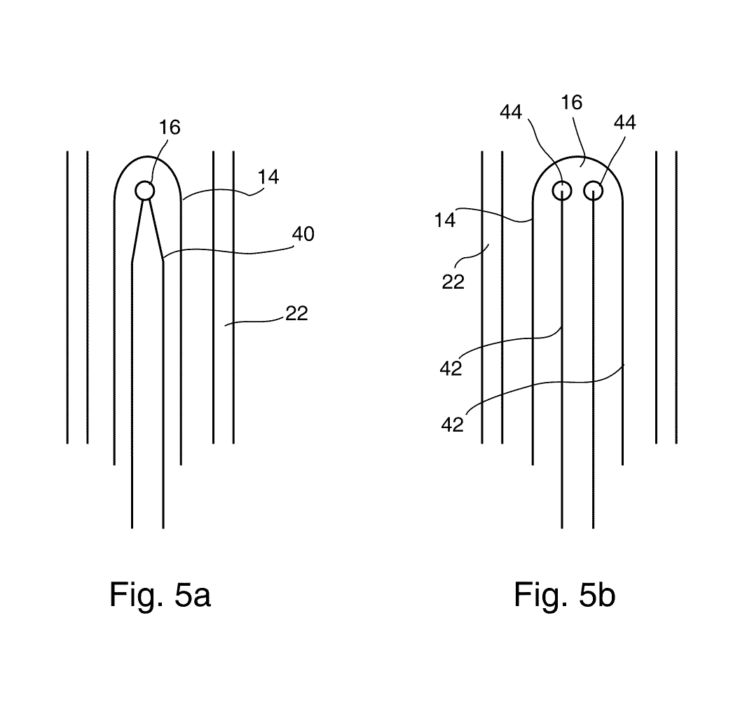

Inflation and guide tube 38 is optionally used to push the end of additional bags 40 (optionally knotted or provided with some other anchoring feature) into the partially set cement or is used to inject an additional anchoring portion of cement 16 as depicted in longitudinal cross section in FIG. 5A. When all desired additional bags 40 have been placed inside longitudinal channel 11, cement 16 is injected substantially as described above. In embodiments when at least one additional bag 40 is placed inside a bag 14, cement 16 is optionally injected so as to flow through perforations in the wall of the bag to also fill up bag 14. Once sufficient cement has been injected to fill the bags, the procedure is completed substantially as described above.

In embodiments of the present invention including multiple bags 14 and 40, the bags are optionally interconnected, for example with filaments or the like to enforce a desired spacing. In some embodiments, the bags are substantially similar. In other embodiments at least some of the bags are different, for example provided with different sized perforations in the walls or including filaments of different strengths.

In some embodiments of the invention, limiting the maximal extent of inflation of a bag (e.g., 14 or 40) allows, by injecting a certain amount of relatively viscous cement, to pretension the tensile elements, increasing the strength of an implant of the present invention. Optionally or alternatively, over injection of cement causes leakage of cement out of the bag. Optionally however, the cement is viscous enough or includes some particles so that some pre-tensioning of the bag is provided as well.

Optionally, in a multi-bag embodiment, at least one of the bags (e.g., an outer or inner one) has pores and another bag does not. Optionally, the pores of different bags are positioned to match up or not match up, depending on the implant design.

In an exemplary embodiment of the invention, at least some of the fibers, for example, that form the bag and/or tensile elements, are formed of a material and/or structure that expands. Such expansion, can be, for example, contact with fluid (e.g., absorption), heat and/or as a result of a chemical reaction with cement.

### Non-Bag Tensile Elements

In some embodiments, the tensile elements are one or more elongated tensile elements, e.g., filaments, monofilaments and multifilaments such as fibers, cables, threads, wires, strings and optionally multifilament tensile elements such as yarns, braids, crochets and knits having a certain limited, degree of axial extensibility when embedded within cement 16 contained within bag 14. Optionally, as described below in greater detail, rods are used.

In an exemplary embodiment of the invention, the extensibility (if any) of the tensile elements is matched with that of the cement to prevent cracking of the cement when they extend. Placement of such tensile elements 42 is optionally substantially as described above for additional bags and may be simplified by the addition of an anchoring element 44 such as an eyelet, a knot or general broadening which is conveniently pushed into the hardening cement as depicted in FIG. 5B. Once sufficient cement has been injected to fill a bag 14 and to fill longitudinal channel 11, the procedure is completed substantially as described above. When tensile elements are pushed (or advanced and then released) into a longitudinal channel 11, for example with a blunt needle or a rod, the blunt needle or rod may have a smaller diameter than a guide tube as such a needle or rod is optionally not configured for injection of cement. In an exemplary embodiment of the invention, the needle encloses the element and is inserted into the bag or implant, optionally to the bag's end. Then, the tensile elements are pushed out, for example, using a pushing rod coaxial with the needle or held in place thus while the needle is retracted. Optionally, the ends of previously inserted fibers remain outside the body (and are held) so that the insertion of the needle does not push in the fibers

Generally, in a situation where sufficient cement has sufficiently hardened to anchor an additional tensile element, or in embodiments employing an anchoring component, see below, the tensile element is optionally pulled so as to pretense the tensile element. The cement is allowed to harden so as to maintain the tensile element in a pre-tensioned state.

Optionally, flexible tension elements include a hook or expanding anchor (e.g., shape memory or super-elastic element) at their tip, to prevent their retraction with the needle which inserts them.

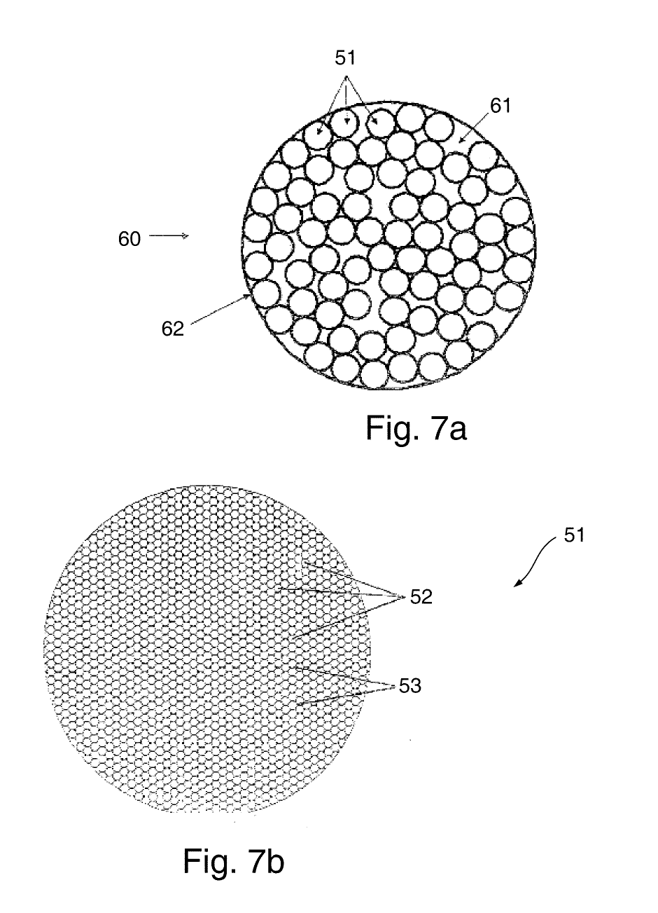

In some embodiments of the invention (FIG. 7), the implant comprises elongated tensile elements, such as rods, which are made of composite material that includes longitudinal, continuous fibers “glued” together or encapsulated within polymer matrix (e.g., PEEK, PMMA, PEKK epoxy, bone cement, Silicone, Polyurethane). An optional not binding exemplary implant 60 cross section is schematically illustrated in FIG. 7A. Elongated rods 51 are relative parallel disposed within a container 62. Bone cement 61 fills the gaps among and around said rods 51. FIG. 7B illustrates a single rod 51, comprising plurality of elongated fibers 52 (for example made of carbon) attached to each other by-/embedded within a matrix 53 (e.g., a polymer such as PEEK, PMMA, PEKK).

In an exemplary embodiment of the invention, the elongated fibers can be one or any combination of the followings: carbon rods or fibers, aramid yarns (e.g. Kevlar or Dyneema), Nylon fibers, PMMA fibers, metal fibers (e.g., Stainless Steel, Titanium, Aluminum, Tungsten alloys), nano tubes. One not binding exemplary tensile element raw material can be the biocompatible ENDOLIGN™ composite material (from Invibio Biomaterial Solution, UK) which is composed of continuous carbon fibers in a PEEK-OPTIMA® polymer matrix. While in some embodiments the elements are composite, in others they are not, for example, being made of metal or PEKK.

In an exemplary embodiment of the invention, the rods may be inserted into a container or may be delivered into bare bone (i.e., no container). Optionally, a channel is created (e.g., drilled) in the bone (optionally in the cortical bone only and parallel to its surface) prior to implant insertion (in a similar manner as described above and below). Optionally, the rods are inserted one-by-one, or optionally one per hole. Optionally, once a desired quantity of said rods are introduced into bone, bone cement (e.g., PMMA, calcium phosphate, calcium sulfate) is introduced into the container (where container is used) or into bone (when container is not used) to fill the gaps between and around the rods. The container (if used) can be permeable (e.g., a bag/mesh), impermeable (e.g., a sealed balloon-like container), or semi-permeable. When a container is used, the bone cement can be used (while enough pressure is applied) to also expand said container while promoting fixation within bone. When a permeable container is used, the delivered bone cement may infiltrate though its walls and promote fixation and/or adhesion to bone interior (e.g., intedigitation into trabecular bone).

In an exemplary embodiment of the invention, the container diameter is optionally about 5 mm, optionally about 8 mm, optionally about 15 mm, or higher or lower or intermediate value. In an exemplary embodiment of the invention, for multi-implant embodiments, a single implant may have the diameter of, for example, 3 mm, 2 mm, 1 mm or smaller or intermediate values, for example, being selected to be less than 50% of a cortical bone thickness.

In an exemplary embodiment of the invention, a tensile element has good tensile resistance capabilities. Optionally, said tensile rod is bendable, for easier insertion and manipulation within bone, while a plurality of such rods, when situated and assembled within bone cavity as an implant, decreases substantially.

In an exemplary embodiment of the invention, the tensile elements (rods) are straight. Alternatively, they are curved or bended in a desired angle and/or a desired location along the rod (e.g., “banana” shape or “J”-shape).

In a preferred exemplary embodiment of the invention, all materials introduced into body are biocompatible. In additional exemplary embodiment of the invention, the container, and/or the tensile element fibers, and/or the tensile element matrix, and/or the bone cement are made of bio-absorbable material, for example the fibers formed a bio-absorbable polymer. In additional embodiment of the invention, additional material and/or medicine may be added to the filling material (for example to bone cement) and/or tensile element rods. In an exemplary embodiment of the invention, antibiotic, and/or osteo-conductive material, and or osteo-inductive material are added to said implant components. Optionally or alternatively, other materials may be added, for example, anti-inflammatories and antibiotics.

In some embodiment of the invention, the procedure(s) described in the above embodiments is performed under imaging devices, such as fluoroscopy. Optionally, radio opaque markers are provided in one or more of tensile elements, container and/or cement.

Alternatively and/or additionally, a guided imaging surgery is performed.

### Straight Bone Channel

Discussed above are some embodiments where a container, curved bag 14, is inserted through insertion hole 24 from the middle of shaft 22 of femur 20 and runs through a longitudinal channel 11 through femoral neck 20.

In some embodiments, a longitudinal channel is substantially straight and passes substantially in parallel only to a shaft 22 of a femur 22 and a straight, optionally rigid, tensile element is implanted therein. Such a longitudinal channel is optionally made in a femur using a straight drill entering the femur from the top of the femur.

In some embodiments a longitudinal channel 101 is substantially straight and passes substantially in parallel only to the axis of femoral neck 20 of a femur 22 and a straight implant (tensile element) of the present invention is implanted therein (FIG. 2 K). Such a longitudinal channel is optionally made in a femur using a straight drill entering the femur from the side of the femur opposite the head of the femur (e.g., lateral side) 102, optionally, over a 3.2 mm diameter K-wire. Alternatively, following insertion of a K-wire, the channel is expanded by, for example, inflation of a balloon (not seen in the Figure). FIG. 2L illustrates a curved longitudinal channel 111 (for example having a “banana” shape) which begins at the lateral side of the proximal femur shaft 112. Optionally, such a curved channel 111, is created using a curved tube, for example made of Nitinol, having a burr at its head which is connected to a rotator shaft (not seen in the Figure). Optionally, the curved tube is guided, for example by a flexible K-wire.

### Multiple Channels

In some embodiments, there are at least two longitudinal channels, optionally crossing for example a first longitudinal channel passing substantially in parallel to the axis of a femoral neck 20 of a femur 22 with a straight tensile element of the present invention is implanted therein (as described above) and a second longitudinal channel that passes substantially in parallel to a shaft 22 of femur 22 with a straight tensile element of the present invention is implanted therein (as described above).

FIG. 2M illustrates two channels 121, 122, formed in the proximal femur, into which two implants are introduced (in a manner as described in the above embodiments). In some embodiments, the upper channel is straight 121, and the lower one is curved 122. Such a combination of two implants may contribute to a better rotational stability of the bone.

In some embodiments, two channels are formed (e.g., one along the trochanter axis and one along the femur axis) and the implant intersects the two channels, for example, by the cement inserted in the two channels and/or tensile elements inserted therein, meeting.

### Bagless Implant

Discussed above are embodiments where longitudinal channel 11 is made in medullary cavity 26 and trabecular tissue. Discussed above are embodiments where cement 16 is at least partially contained within a container such as a bag 14 components of which that constitute at least some of the tensile elements of an implant of the present invention. In some embodiments, an implant is devoid of a container such as bag 14.

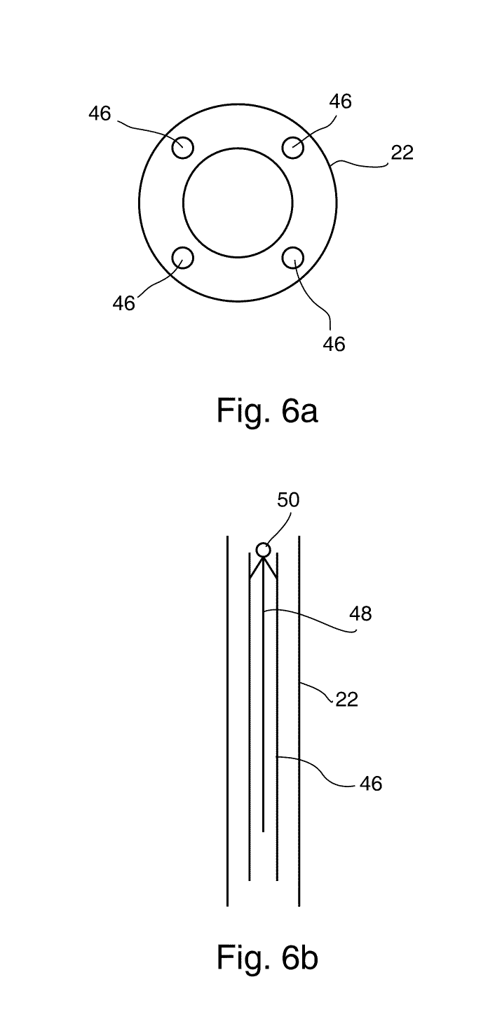

In some embodiments an implant (comprising a container and/or other tensile elements) is implanted inside a longitudinal channel that runs, at least in part, through cortical bone. An exemplary such embodiment is depicted in axial cross section in FIG. 6A and in longitudinal cross section in FIG. 6B.

In FIG. 6A are seen four longitudinal channels 46, each, for example, 0.5 mm in diameter running substantially parallel to the axis of a femoral neck 20 through the compact bone tissue constituting the walls of femoral neck 20. In FIG. 6B is seen a single longitudinal channel 46 in longitudinal cross section in which a linear tensile element 48, (e.g., braided aramid filaments 0.2 mm in diameter) of an implant of the present invention is disposed. Linear tensile element 48 is optionally held in place with the help of an anchoring plug 50, which includes two outwardly biased elastic members of stainless steel in a chevron conformation and optionally plugs the surrounding cavity.

Forming 104 longitudinal channels 46 is optionally using straight bone drilling, for example, using a straight narrow drill head drilling into the femur from the thigh opposite the head of the femur.

Inserting tensile element 48, involves, for example, pushing anchoring plug into a longitudinal channel 46 with, for example a stiff rod. Optionally, the elasticity and chevron arrangement of the elastic members of anchoring plug 50 allows plug 50 to be easily pushed into a longitudinal channel 46 but resists withdrawal therefrom.

When an anchoring plug 50 is pushed sufficiently far into a longitudinal channel 46, cement 16 is injected into longitudinal channel 46, substantially as described above, a step substantially equivalent to a step 108 of inflating a container. Once sufficient cement has been injected to fill the bags, the procedure is completed substantially as described above. Optionally, a same needle is used to insert the tensile element and inject cement. Optionally, the needle includes a bone drilling head and is also used to drill the channel for the thread

Pre-tensioning a tensile element 48 such as a tensile element described in FIGS. 6A and 6B is optionally performed by pulling on a tensile element until cement 16 has sufficiently set.

In embodiments, a tensile element, whether a container such as a bag or a elongated tensile element such as a fiber or filament may be radially tamped against the sides of a longitudinal channel with the help of a ram or inflatable element.

### Water Jet Drilling

In an exemplary embodiment of the invention, drilling and/or reaming and/or cutting of bone tissues (trabecular and/or cortical) are performed by water-jet technique. Using water-jet techniques for bone surgeries is described, for example, in Schwieger et al (2004), “Abrasive Water Jet Cutting as a New Procedure for Cutting Trabecular Bone—In Vitro Testing in Comparison with the Oscillating Saw”; and Honl et al (2003), “The water jet as a new tool for endoprosthesis revision surgery—An in vitro study on human bone and bone cement”; the disclosures of which are fully incorporated herein by reference. The two articles suggest using water, optionally with abrasive material (preferably biocompatible), for drilling small holes (similar to jet diameter) or cutting bones. In an exemplary embodiment of the invention, the holes created are substantially larger than jet diameter. Optional holes diameter is about 5 mm, optionally about 10 mm optionally about 15 mm, or lower or higher or intermediate value. Optional jet diameter is about 0.05 mm, optionally about 0.1 mm, optionally about 1 mm, or lower or higher or intermediate value. Optionally, the ratio of diameters of hole and jet is in the range of 10:1-1000:1, optionally about 100:1.

In an exemplary embodiment of the invention, at least two water jet sources are provided. Optionally, the at least two sources are radially distant one from the other, to support a hole formation larger than the jet, for example, of order of the distance between the jets, which is substantially larger that individual jet stream projected diameter. Optionally, the at least two jets can be (in a fixed and/or controllable manner) pointed perpendicularly to/into the bone, or can be slightly tilted inwardly (towards each other), or alternatively be tilted outwardly. The coupled jet sources are optionally rotated around a central axis thus performing needed drilling or reaming.

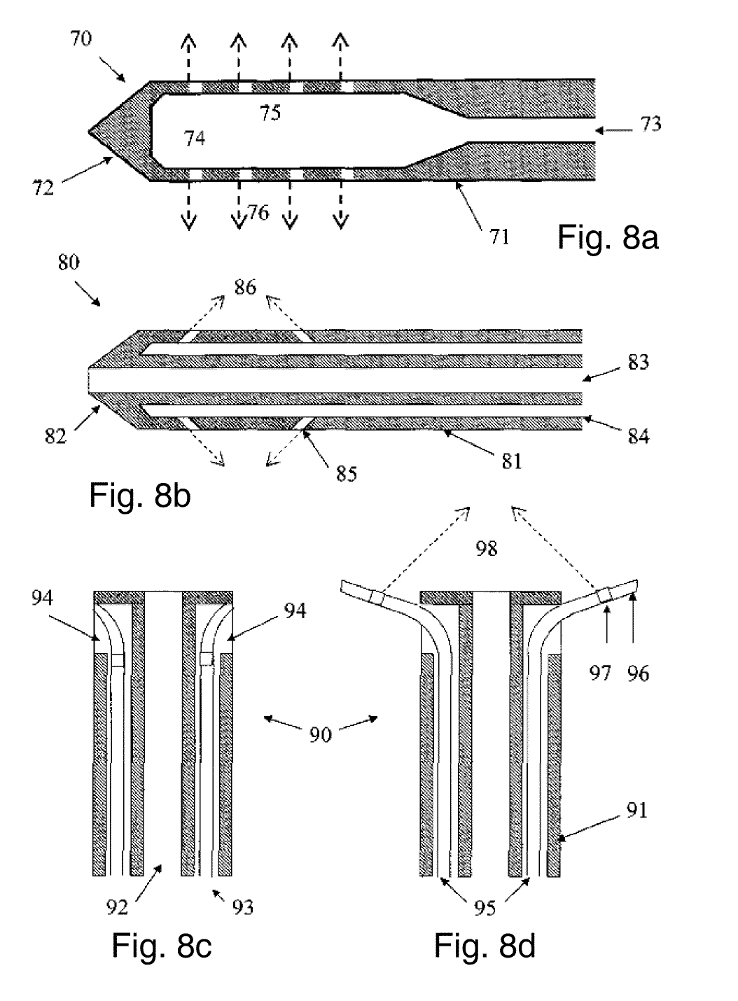

FIGS. 8A-8D illustrate three different exemplary drilling heads which incorporate water jet drilling/cutting techniques.

FIG. 8A illustrates a cross section of a hollow tubular drill head 70 having driller body 71, drilling tip 72, fluid inlet 73 located on its proximal side, fluid basin 74 and a plurality of openings for fluid-jet ejection. In a preferred exemplary embodiment of the invention, driller 70 is rotated along its longitudinal axis and pushed distally into or within bone, such that drilling tip 72 can drill a hole having a diameter substantially equal to its largest diameter. An exemplary hole diameter may be in the range of 0.5-5 mm. Optionally, before, during and/or after drilling operation performed by rotation of drilling tip 72, a fluid is pressurized though inlet 73 towards basin 74, thus a plurality of pressurized fluid jets 76 emerges laterally through openings 75. In a preferred exemplary embodiment of the invention, fluid jets 76 have enough impact to cut/engrave through bone tissues surrounding driller body 71, thus enabling hole enlargement (with respect to an initial hole made by drill head 72, and as described in greater detail below) while driller 70 advances within bone. An exemplary enlarged hole diameter may be in the range of 5-15 mm.

FIG. 8B illustrates a different exemplary drill head 80 having hollow drilling tip 82, driller body 81 with two concentric lumens: inner lumen 83 which is in communication with tip 82 and outer lumen 84 which is in direct communication with openings 85. As in driller 70, when rotating driller 80, drilling tip 82 can be used to perform a hole having initial smaller diameter, to be enlarged by fluid jets 86 that are injected through openings 85 by pressurizing fluid introduced through outer lumen 84. As schematically illustrated, the at least two pairs of adjacent openings 85 may optionally be tilted toward each other. Optionally, lumen 83 may be used for riding the drill over a guide wire and/or a small diameter drilling element (not shown), previously introduced into bone. Optionally or alternatively, lumen 83 may be used for providing a cutting jet. Optionally or alternatively, lumen 83 may serve as a channel for other instrumentation(s) (not shown) to be introduced before, during or after drilling takes place. Said instrumentation(s) may optionally include, but are not limited to, collecting or suction devices for removing cut bone chips, and/or may act as a lumen for suction applied from outside.

FIGS. 8C and 8D illustrate two operational modes of a third exemplary drill head 90. As in driller 80, driller 90 contains driller body 91 having two concentric lumens: inner lumen 92 which has similar functionality to inner lumen 83, and outer lumen 93, which is in direct communication with at least two lateral openings 94. Alternatively, driller 90 has three parallel (non-concentric) lumens. In an exemplary embodiment of the invention, there are at least two bendable pipes 95, optionally metal, located in lumen(s) 93. Each pipe 95 optionally includes a distal end 96 which optionally incorporates mechanical bone cutting capabilities, and at least one fluid injection port 97, which is optionally tilted forward of driller 90 to its axis, when the pipes are extended. FIG. 8C illustrates driller 90 in a closed mode while FIG. 8D illustrated driller 90 in an opened mode: in the closed mode pipes 95 are substantially within lumen(s) 93, while in the opened mode pipes 95 are protruded outwardly. In an exemplary embodiment of the invention, while driller 90 is rotated, fluid jets 98 are injected distally in front of driller 90 through ports 97, cutting distally into bone, and pipes 95 distal ends 96 serve as a lateral cutting heads for enlarging the diameter of a hole created by jets 98.

### Expanding Drill Head

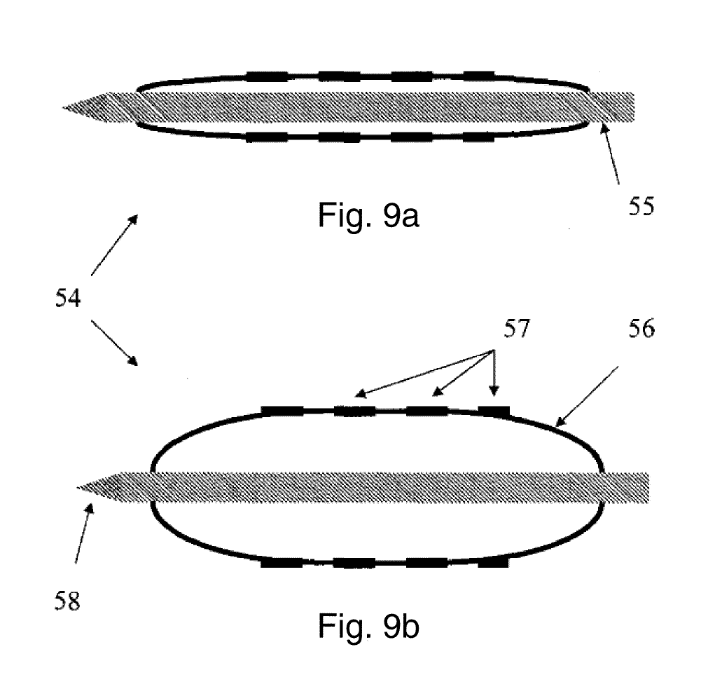

FIGS. 9A-9B illustrate another exemplary excavation component which comprises of expandable and/or inflatable circumferential element that enables selection of different working diameters.

FIG. 9A shows a cross section view of exemplary driller 54 having core 55, which its distal end 58 is preferably tipped, and an inflatable and/or expandable cover 56, which is textured and/or roughened and/or covered with abrasive material as schematically illustrated by texture 57. Texture 57 may include but is not limited to abrasive materials such as diamond powder/granules, silicone carbide powder, sugars (i.e., lactose), salts and minerals, and/or any other, preferably biocompatible, abrasive powders and/or may include small cutting edges, such as bone knifes.

FIG. 9A presents driller 54 in a closed mode, whereas FIG. 9B presents driller 54 in an opened and/or partially opened mode. In an alternative exemplary embodiment of the invention, driller 54 distal end includes only an inflatable and/or expandable element.

In an exemplary embodiment of the invention, when in closed mode, driller 54 may act as mechanical driller able to perform holes in nominal sizes (usually, though not limited to, a diameter within the range of 0.5-5 mm). When in opened mode, i.e., when driller 54 is expanded to a preferred diameter (usually, though not limited to, a diameter within the range of 5-15 mm), driller 54 is manipulated within bone by peeling techniques such as by rotation and/or inward-outward maneuvering of the drill, while its textured surface will be pressed against the hole inner diameter, in order to promote its widening.

In an exemplary embodiment of the invention, cover 56 is a balloon inflated using a fluid lumen (not shown) optionally communicating between an outside fluid pressure source and an opening inside of the balloon. In an alternative embodiment, cover 56 comprises a plurality of strips, which bulge out when their ends are moved closer together, for example, by one end of the strips being connected to core 55 and the other end to a telescoped overtube (not shown), whose axial position relative to core 55 is controllable.

### Exemplary Process and Tool Set for Rod-Based Implant

As noted above, in an exemplary embodiment of the invention, at least some of the tensile properties of the implant are provided by a plurality of rods. Following is a description of a method and tool set for such an implant, in which the drilling is substantially in a straight line. It should be noted that the various features, tools and/or property values described herein are also applicable to other embodiments described herein and vice versa.

### Overview

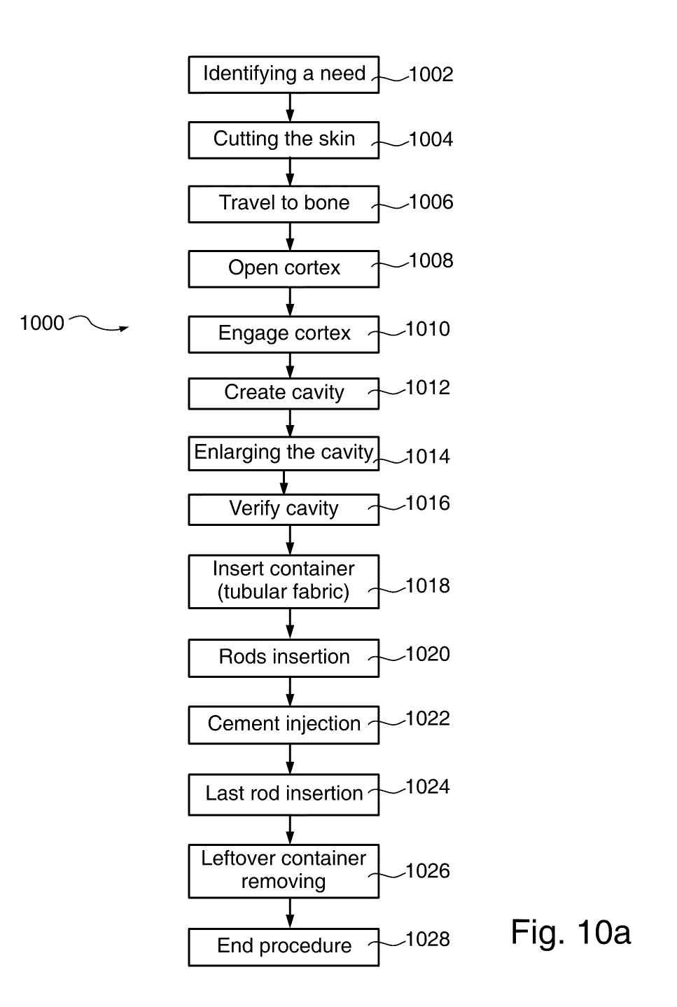

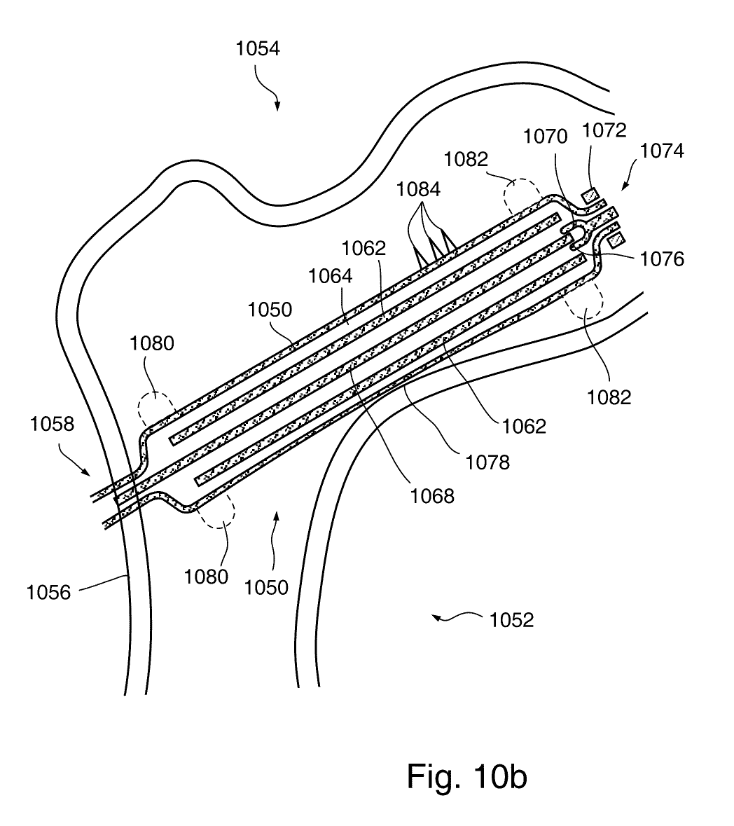

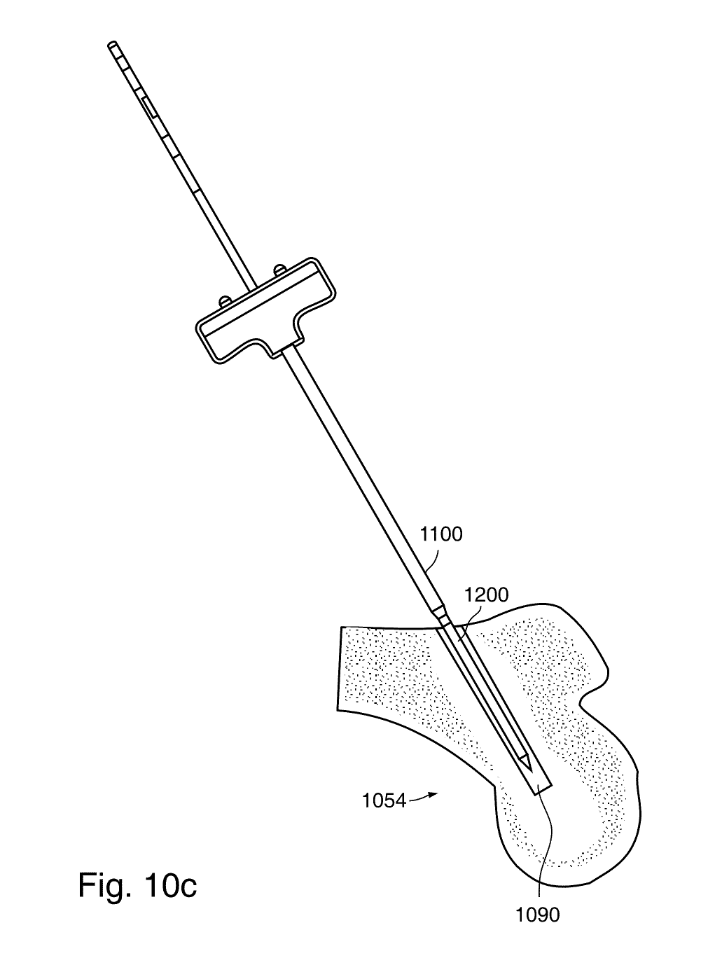

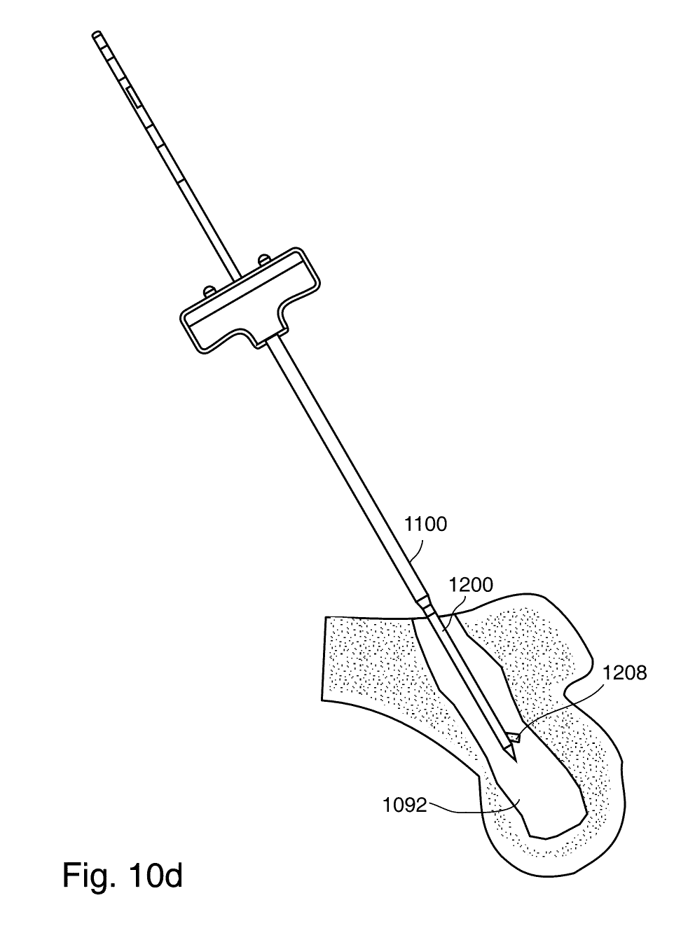

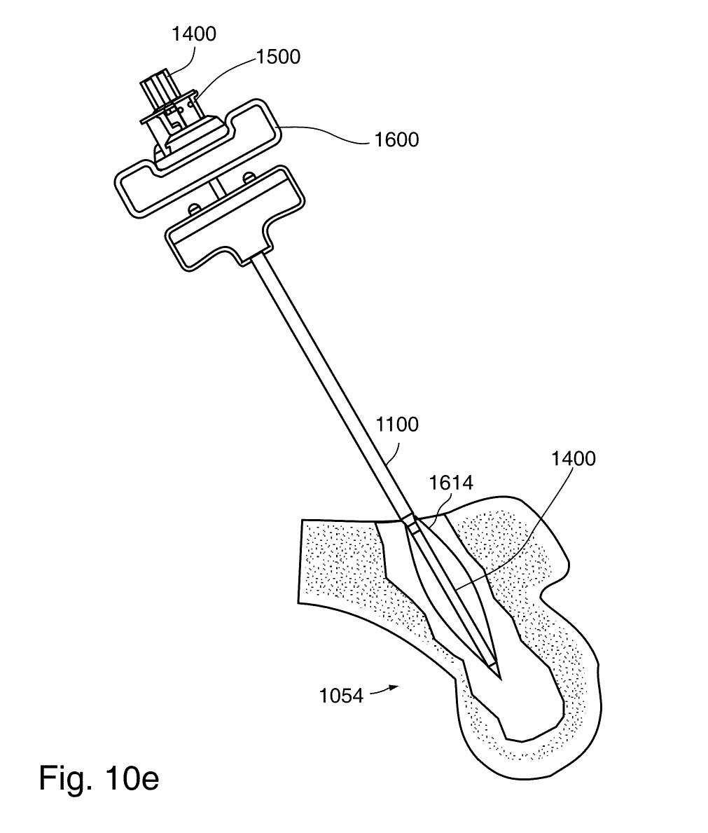

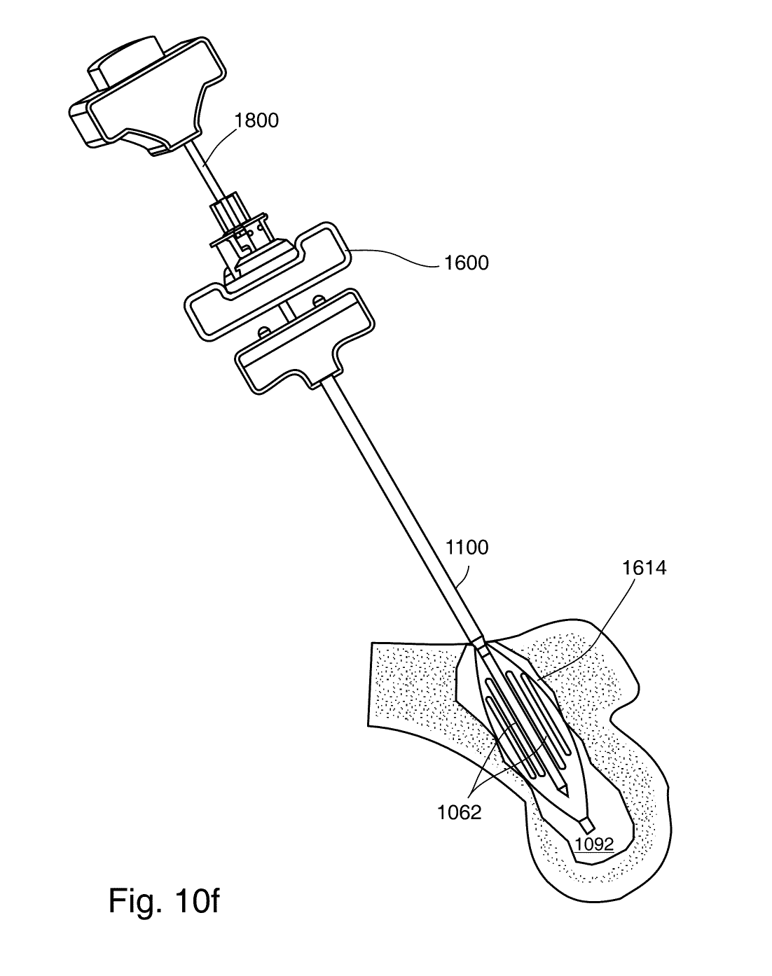

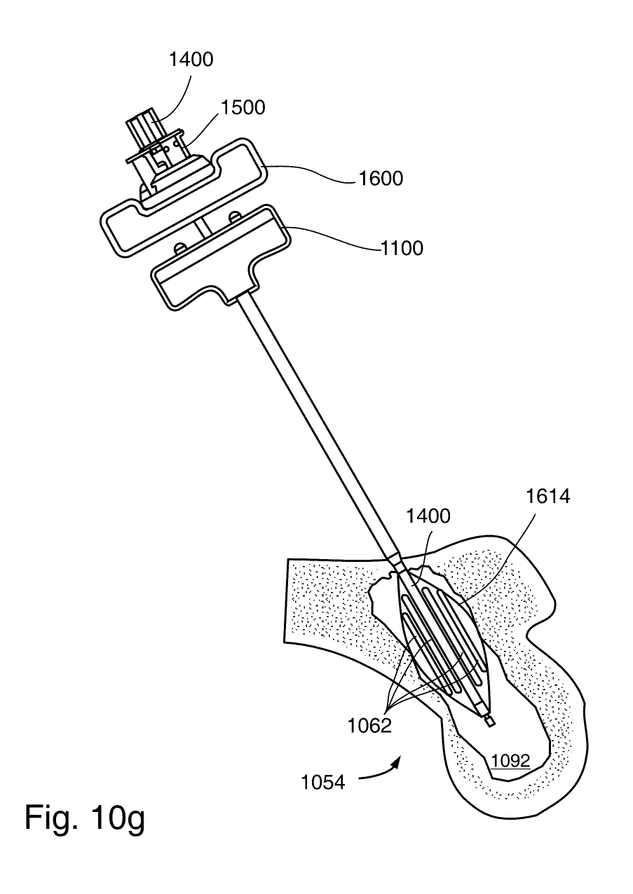

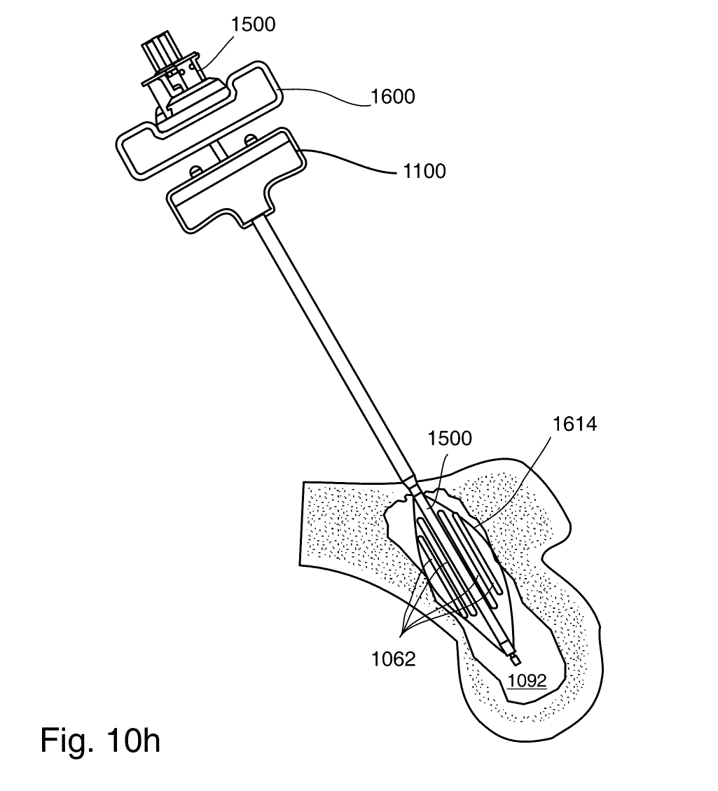

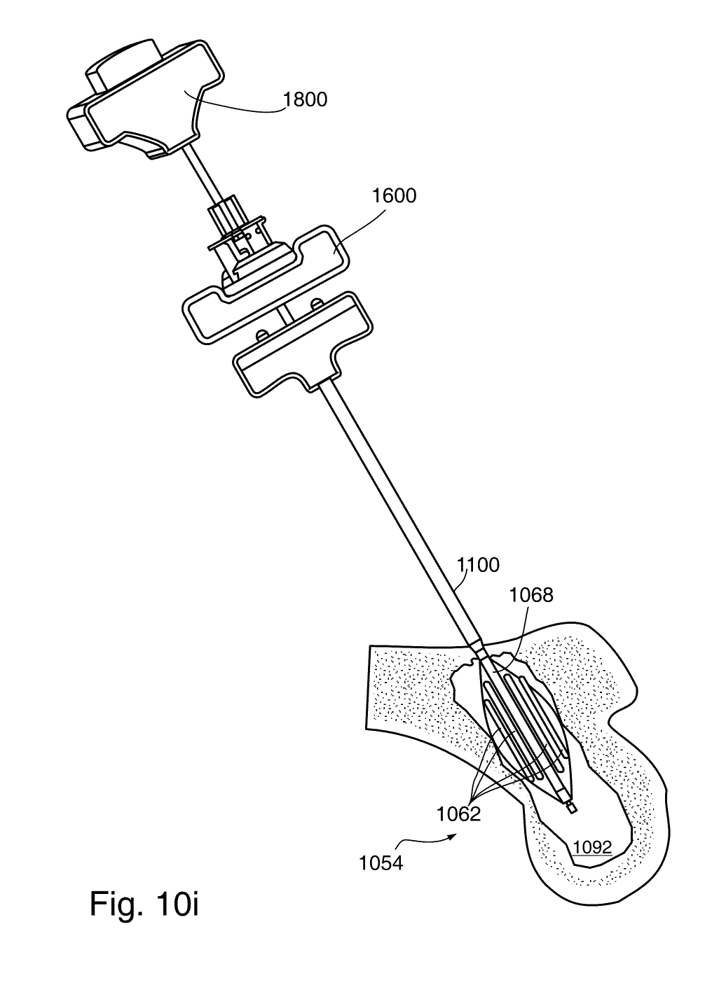

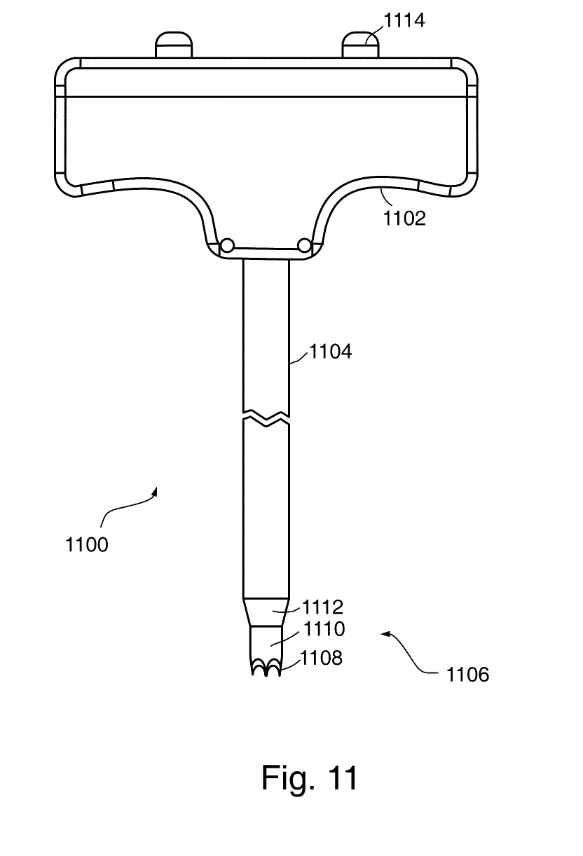

FIG. 10A is a flowchart of a method 1000 of composite bone implant construction and implantation, in accordance with an exemplary embodiment of the invention. FIG. 10B is a cross-sectional view of a composite implant 1050 in a femur 1052, in accordance with an exemplary embodiment of the invention. FIGS. 11-19 illustrate components of a bone implant kit usable for the method of FIG. 10A, in accordance with an exemplary embodiment of the invention.

FIGS. 10C-10I illustrate snapshots of points along an exemplary process shown in FIG. 10A.

In an exemplary embodiment of the invention, for the treatment of proximal femoral fractures, a channel having a diameter of, for example, 4 mm, or 5 mm, or 6 mm, or 8 mm, or 10 mm, or 12 mm is made, from the lateral side of the proximal femur shaft, through the femoral neck, up to the femoral head. In an exemplary embodiment of the invention, an insertion passage is created in bone, for example a K-wire (having a diameter of, for instance, 3.2 mm) is introduced. Over the K-wire, an insertion tube is optionally introduced (followed by the removal of the K-wire). Optionally, the tube is inserted in a different manner than over a K-wire. Then, a bag container is introduced and positioned in said tube and the tube is retrieved.

A balloon device is then optionally inserted into the container and inflated, in order to expand the channel (and the container) within the bone.

In an exemplary embodiment of the invention, the channel is expanded using a special K-wire. For example, the K-wire can include a side extending element that when extended serves to cut or otherwise break down trabecular bone and/or cortical bone, when the k-wire is rotated and/or moved axially. Optionally or alternatively, the K-wire has an expandable tip or an eccentric tip, for example, as described in PCT publication WO2005/032326, the disclosure of which is incorporated herein by reference. In some embodiments, the side extending element is made rigid enough to cut trabecular bone but bends when contacting cortical bone, possibly reducing damage to cortical bone.

Following this optional expansion, the balloon device is deflated and retrieved, and elongated tensile elements, for example rods of composite material such as carbon fibers embedded in polymer matrix, are introduced into the bag container. After a satisfactory filling of the bag with said rods, filling material such as bone cement is injected into the bag to fill the gaps between and surrounding the rods.

Alternatively, following creating a passage, a balloon device is introduced into the tube/bone passage prior to bag container insertion. The tube is removed and the balloon is inflated, deflated and retrieved, followed by introduction of a bag container. Then, elongated tensile elements are introduced and filling material is injected as described above.

In an alternative exemplary embodiment of the invention, following removal of the K-wire, a balloon, optionally a balloon that has a mesh embedded in it for using high pressure for example 100 Bar, covered by a bag container, is inserted into the insertion tube. Said tube is retrieved, and balloon is expanded. Then, elongated tensile elements are introduced and filling material is injected as described above. Alternatively, the bag is inserted after the balloon is removed.

Referring specifically to FIG. 10B, when the implant construction process is completed, implant 1050 is optionally as follows. A bag 1060 encloses a plurality of rods 1062 and cement 1064 between the rods. An optional elongate rod 1068 reaches along the entire length of bag 1060, from an aperture 1058 formed in a cortex 1056 of hip 1052 to a distal end 1074 of bag 1060. Optionally, end 1074 is reinforced using a metal seal 1070 that includes a hollow 1076 for receiving a rod and an optional outer band 1072 for locking seal 1070 to bag 1060. Implant 1050 is shown generally parallel and along an axis of a trochanter 1054. Optionally, implant 1050 leans on the cortex of trochanter 1054 at a point 1078. Optionally, implant 150 rests on cortex at aperture 1058 and point 1078.

Optionally, bag 1060 includes apertures along its length so that some cement can leak and form one or more inter-digitations 1084 (only a few shown) to enhance engagement of trabecular bone. Optionally or alternatively, bag 1060 includes additional apertures adapted to support greater cement leakage, for example at a proximal end (forming anchoring section 1080) and/or at a distal end (forming anchoring section 1082).

### Exemplary Tool Set

FIGS. 11-19 show an exemplary tool set, with matching handles and wherein all the tools fit through a cannula. Alternative designs are described after. In addition, it should be noted that some or all of the tools may be replaced by other tools and still be used to carry out methods in accordance with an exemplary embodiment of the invention.

In an exemplary embodiment of the invention, the tools are provided as a kit, optionally in a sterile package, optionally with instructions. The implant components (e.g., bag, cement, rods) may be provided in a same kit or provided separately. Optionally, the tools are sterilizable, however, hardening of bone cement may prevent reuse of at least some of the components.

In an exemplary embodiment of the invention, the tools are rigid, for example, formed of stainless steel. Optionally, at least some of the tools are flexible and optionally provided via a flexible cannula or endoscope. For example, the drill, cannula, bag holder, stylet, bag cutter, element carrier and/or cement cannula may be flexible. Some or all of these tools may be formed of non-metals, for example, plastic, PEEK, PEKK and/or Composite materials.

### Bone Cannula

FIG. 11 illustrates a bone access cannula 1100, optionally used for accessing the bone to be treated, in accordance with an exemplary embodiment of the invention. Cannula 1100 includes a shaft 1104 mounted on an optional handle 1102. Optionally, one or more engagement elements 1114 are provided for interlocking with other components of the tool set.

At a distal end 1106 of shaft 1104, a bone cutter 1108, for example, a serrated edge, adapted for digging into cortical bone is optionally provided. A straight guide section 1110 is provided proximal thereto and optionally sits across cortical bone, in use. A widening cone 1112, or other widening, optionally serves to limit advancement of top 1106 into bone.

In an exemplary embodiment of the invention (for hip), the cannula has a diameter of 6.9 mm diameter and length of 160 mm, optionally formed of stainless steel working sleeve. The tapered portion is optionally of a diameter of 4.8 mm and the inner diameter is optionally approximately 4.2 mm. The handle is optionally polycarbonate handle.

It should be noted that smaller sizes may be used in other bones, and depending on various design variations, for example, as described below.

### Bone Drill

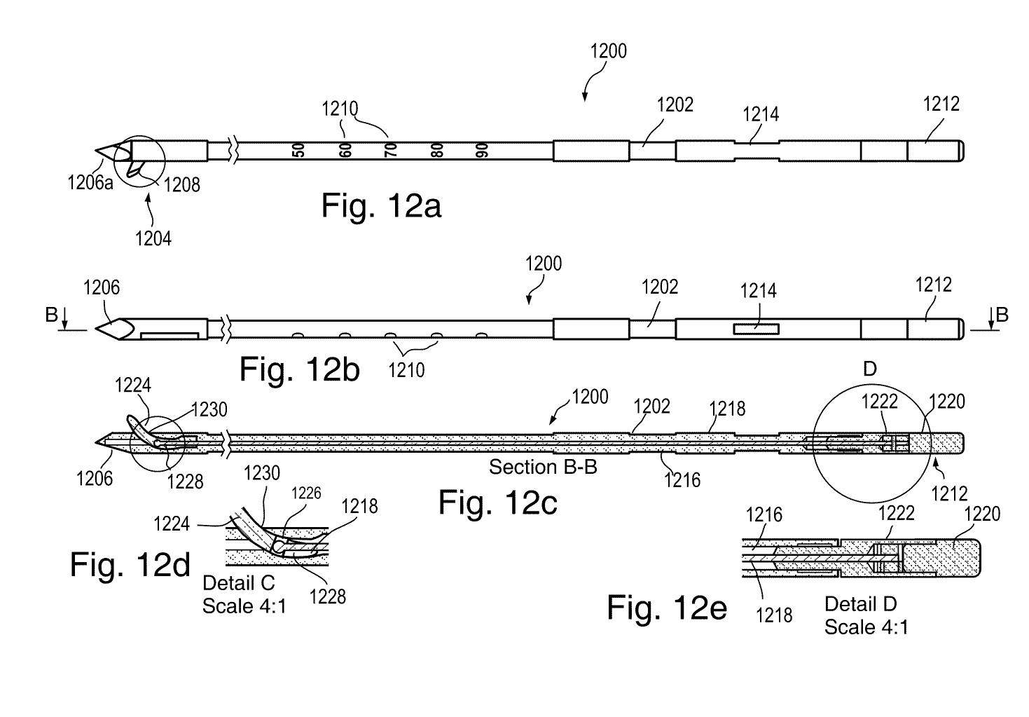

FIGS. 12A-12E illustrate a bone drill 1200, optionally used for piercing through soft tissue, drilling though cortical bone, drilling through trabecular bone and/or widening a pathway in trabecular bone or/and cortical bone, in accordance with an exemplary embodiment of the invention. FIGS. 12A and 12B show different side views of drill 1200.

Drill 1200 has a shaft 1202 optionally sized to fit in cannula 1100. A distal end 1204 optionally includes a bone cutting tip 1206 adapted for drilling into cortical bone, and also usable for trabecular bone. An optional side extending cutter 1208 is also shown which is selectively extendible to widen a trabecular or/and cortical bone channel and when retracted, does not affect the drill diameter.

A plurality of markings 1210 is optionally provided. The markings are optionally radio-opaque. Optionally, the markings are outside the body and used to indicate the relative position of distal tip 1204 relative to the distal tip of cannula 1100.

A recess 1214 is optionally provided for attachment of a handle and/or a motor (e.g., if a hand-held drilling motor and handle are provided with a bone treatment kit).

A proximal end 1212 is optionally provided for controlling the extension of cutting element 1208, by rotation of a knob 1220. Other mechanisms may be used as well.

FIG. 12C is a cross-sectional view of drill 1200, and FIG. 12E a detail of proximal end 1212, showing a threading 1222, which engages knob 1220. Knob 1220 is coupled to a shaft 1218 which lies in a lumen 1216.

Referring also to FIG. 12D, a detail of distal end 1204, a curved knife element 1224 of cutting element 1208 exits through an aperture 1230 and is engaged by shaft 1218 using a rotating joint 1226. A space 1228 is optionally designed to receive element 1224 when retracted. In this embodiment, element 1224 optionally does not change shape during extension/retraction. In an exemplary embodiment of the invention, element 1224 extends sideways after being pushed along a rail-like mechanism extending to outside the body. In embodiments where element 1224 changes shape, it maintains its rigid shape after the extrusion through aperture 1230.

In an exemplary embodiment of the invention, drill 1200 has a 4.2 mm diameter, and a length of 350 mm. Optionally, drill 1220 is formed of stainless steel. This and/or other tools may be formed of other materials (optionally flexible), including, for example, Carbon—PEEK, plastics and composite materials or other metals. Cutting element 1208 optionally extends up to 3 mm away from the surface of shaft 1202, thereby providing a total drilling diameter of up to 10 mm diameter.

In an exemplary embodiment of the invention, tip 1206 of drill 1200 operates when drill 1200 is rotated in either direction. Alternatively or additionally, extending element 1208 operates when drill 1200 is rotated in either direction.

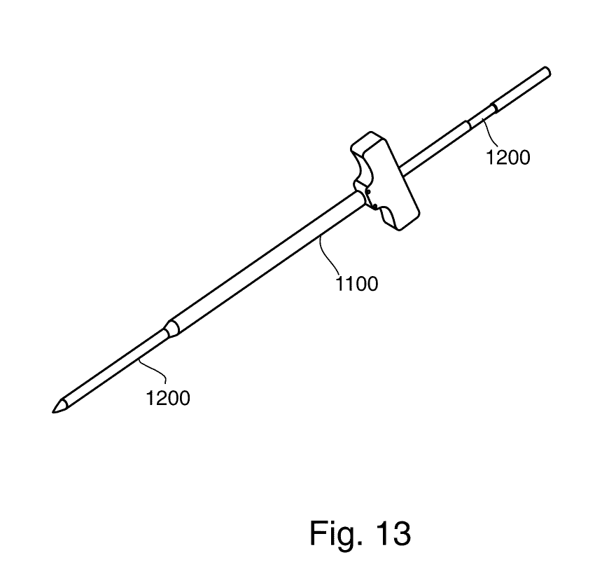

FIG. 13 illustrates the bone drill of FIGS. 12A-12E mounted in the bone access cannula of FIG. 11, in accordance with an exemplary embodiment of the invention. In particular, side cutting element 1208 is retracted, so dill 1220 fits through cannula 1100.

### Stylet

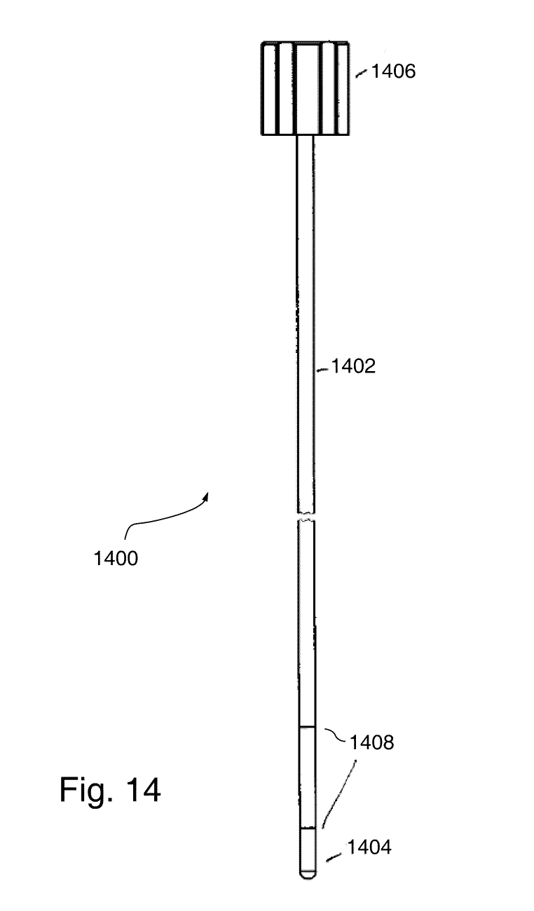

FIG. 14 illustrates a stylet 1400, optionally used to maintain a shape of a cement injection cannula and/or of a mesh bag, in accordance with an exemplary embodiment of the invention.

Stylet 1400 has a shaft 1402, an optional knob 1406 for manipulation thereof and a rounded tip 1404. Optionally, one or more steps 1408 serve as radio-opaque markers. Other marker types may be used.

In an exemplary embodiment of the invention, the stylet is 2.2 mm in diameter and in length of between 145-195 mm. Optionally, a plurality of sizes are provided, to match different implant lengths. Optionally, the sizes are in 10 mm increments. A plurality of sizes may also be provided for drill 1200. Optionally or alternatively, movable stops (not shown) are provided on the shafts of one or both of stylet 1400 and bone drill 1200, to prevent over insertion into the body. Optionally, the desired depth of penetration is determined by inspecting x-ray images of the treated bone, before and/or during treatment.

### Cement Delivery

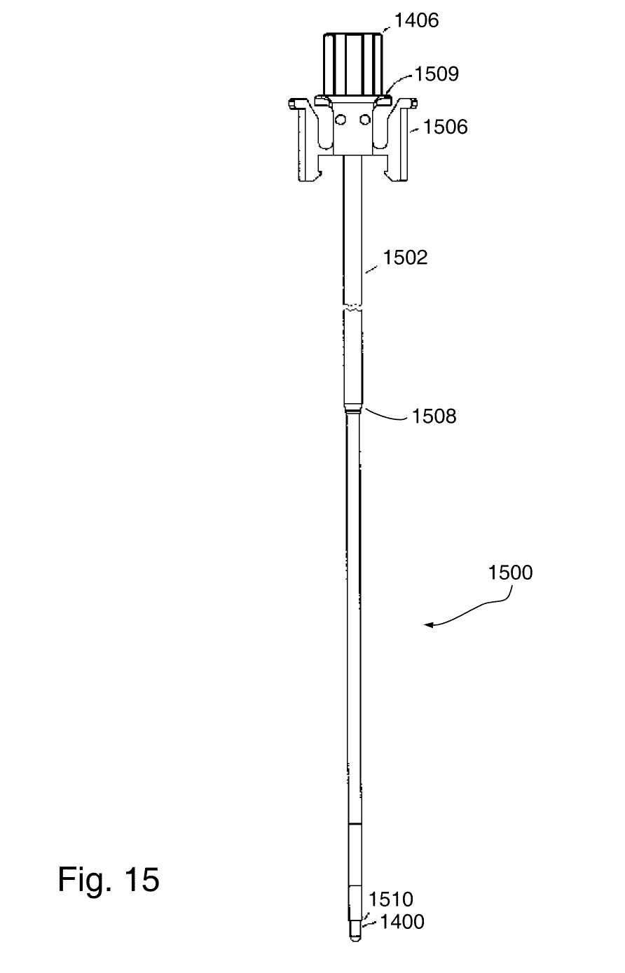

FIG. 15 illustrates a cement delivery cannula 1500, optionally used for delivering cement into the implant, with stylet 1400 mounted therein, in accordance with an exemplary embodiment of the invention. In an exemplary embodiment of the invention, cannula 1500 includes a shaft 1502, a cement injection port 1504, adapted for attachment to a cement/pressure source and an optional lock 1506 for locking to cannula 1100. Optionally, the cement pressure source (not shown) is a syringe and/or a hydraulic cement pump.

In some embodiments, cement is provided directly through cannula 1100 without an additional cement delivery cannula. Where provided, cannula 1500 may be short, reaching only to a proximal side of the implant, for example, reaching to a forward tip 1508. In other embodiments, cannula 1500 reaches to a distal end of the bag, for example, to a tip 1510.

In an exemplary embodiment of the invention, cannula shaft 1502 has a diameter of between 3.4 mm and 2.62 mm. Optionally, the variation in diameter (within a device) is used for one or both of blocking cement backflow (see 1508 below) and/or providing pushability to cement cannula 1500. Optionally, the length of the cannula varies according to the usage. Optionally, the diameter narrows along the cannula, starting, for example, at 3.4 mm and narrowing to 2.2 mm at point 1508, which is optionally designed to match the narrowing in cannula 1100. Optionally, the use of a wider diameter section allows resistance to cement injection to be reduced.

In an exemplary embodiment of the invention, stylet 1400 inserted inside cannula 1500 before the injection to keep cannula 1500 straight.

### Bag Holder

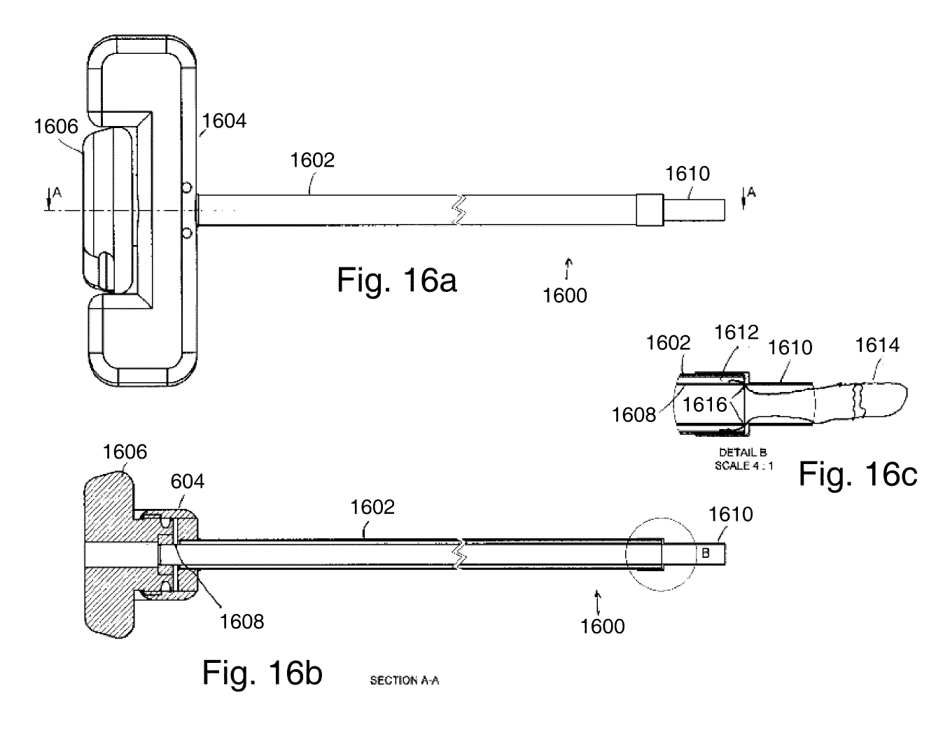

FIGS. 16A-16C illustrates a bag holder 1600, used for holding a mesh bag and providing access thereto during the implantation procedure, in accordance with an exemplary embodiment of the invention.

In an exemplary embodiment of the invention, the mouth of the bag is held between two tubes, optionally of stainless steel. Axially separating the tubes releases the bag. Alternative holding and releasing methods are described below.

FIG. 16A is a side view and FIG. 16B is a cross-sectional view of holder 1600. FIG. 16C is a detail of the bag holding mechanism.

Holder 1600 has a handle 1604 coupled to a shaft 1602 that acts as an outer tube. A second handle 1606, optionally lockable to handle 1604 is coupled to an inner tube 1608. the bag (1614) is held between tubes 1602 and 1608. A sleeve 1610, which is optionally sized to enter into the bone or at least cross the cortex, optionally serves as a guide into the bag and optionally assists in aiming rods 1062 as they are inserted.

Referring specifically to FIG. 16C, bag 1614 is pinched at a point 1616 between distal ends of tubes 1608 and 1602, where there is a narrowing (e.g., a step narrowing as shown, or a gradual narrowing) of tube 1602. A neck of bag 1614 is optionally located in a space 1612 between the tubes

In an exemplary embodiment of the invention, holder 1600 has an outer diameter of 4.2 mm, so it fits inside cannula 1100, in an extra-bone portion thereof. Outside diameter of 4.2 mm, length of 180 mm.

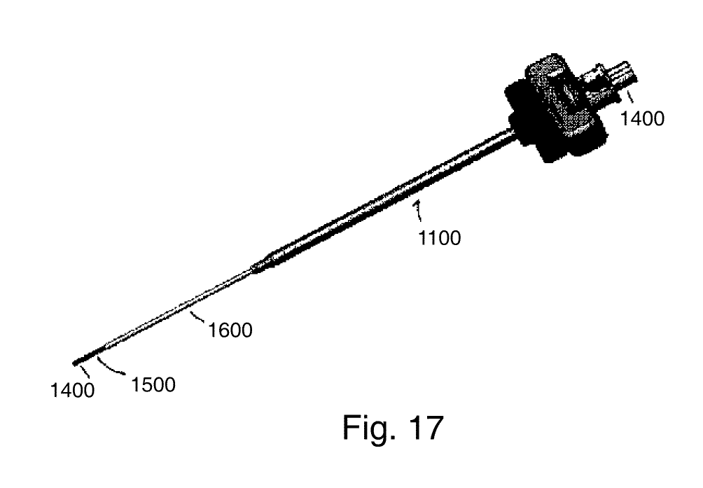

FIG. 17 illustrates stylet 1400, mounted in cement delivery cannula 1500, mounted in bag holder 1600, all mounted in bone access cannula 1100, in accordance with an exemplary embodiment of the invention. Optionally, stylet 1400 is used to hold the bag straight during insertion. In other embodiments, a rod 1062 is used as a stylet for inserting the bag.

### Rod Carrier

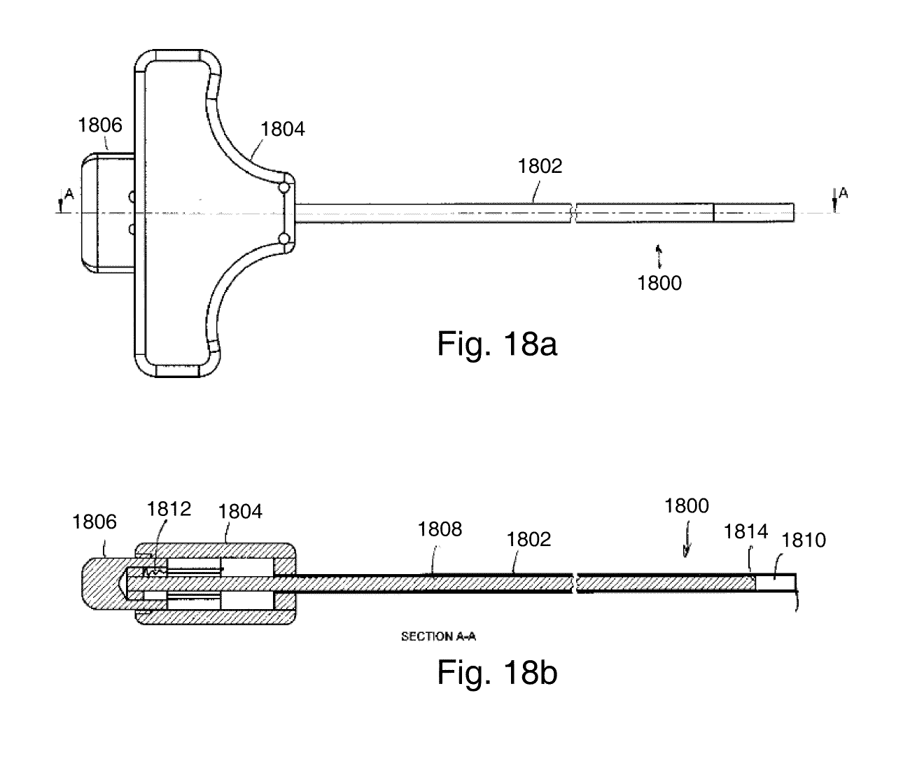

FIGS. 18A-18B illustrate a rod carrier 1800, used for inserting and releasing rods 1062 into bag 1614, in accordance with an exemplary embodiment of the invention. FIG. 18A is a side view and FIG. 18B is a cross-sectional view.

In an exemplary embodiment of the invention, rod 1062 is held by a tight fit or friction inside a lumen of a shaft 1802 of carrier 1800, resting against a narrowing in the lumen (which may have a gradually narrowing inner cross-section, when advancing from distal end proximally) and/or against a tip 1814 of a piston 1808. A short section 1810 of shaft 1802 serves as a holder for the tip of rod 1062. To release, piston 1808 is advanced and/or tube 1802 retracted while maintaining piston 1808 in place, such that the proximal end of rod 1062 is released.

In an exemplary embodiment of the invention, tube 1802 is coupled to a handle 1804 and piston 1808 is coupled to a push-button or other actuator 1806, which is optionally coupled by a spring 1812 to handle 1804.

Optionally, the diameter of shaft 1802 is 3.4 mm and the length is 280 mm. Optionally, a forward tip 1816 of carrier 1800 does not enter into the bone and/or does not enter past the cortex. Alternative designs are shown below.

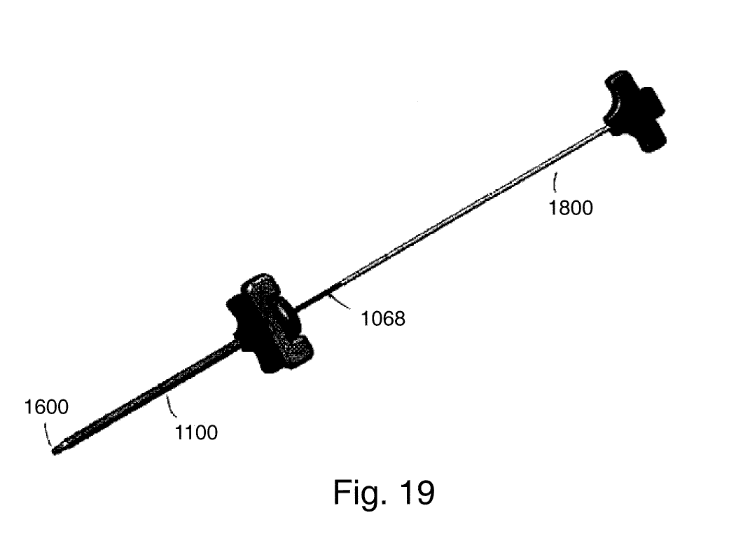

FIG. 19 illustrates rod carrier 1800, mounted in bag holder 1600, all mounted in the bone access cannula 1100, in accordance with an exemplary embodiment of the invention.

Described below, after the process, in greater detail are bag 1614 and rods 1062.

### Exemplary Implant Construction Process

Referring back to FIG. 10A, the process of constructing an implant in situ in accordance with exemplary embodiments of the invention and using the tool set just described, is now detailed.

### Identify a Need (1002)

In an exemplary embodiment of the invention, the need is identified based on several factors including but not limited to: bone illness and porosity, patient age and medical history, the current state of the opposite/twin bone.

In an exemplary embodiment of the invention, the need answered is a cracked or weakened trochanter. In some cases, this is identified by x-ray, by ultrasound and/or by fractures in other bones. Optionally or alternatively, identification is by a general measure, such as osteoporosis and/or age. In an exemplary embodiment of the invention, when a patient exhibits a fracture or other bone damage in one limb, this is taken as an indication that a treatment should be carried out for the mirroring limb. A same or different treatment may be provided to the obviously damaged limb.

In an exemplary embodiment of the invention, due to the reduced invasiveness, a reduced amount of painkillers and/or anesthesia may be used, and/or a lower level of anesthesia, for example a local nerve block. Optionally, no unconsciousness is required.

In an exemplary embodiment of the invention, a candidate patient for a preventive surgical treatment as described herein is an aged person (over 60 years old) with moderate osteoporosis specifically identified in his/her femoral bones, and with a first fractured and/or cracked femur and a second femur optionally not fractured or cracked. In such an exemplary case, both femurs or at least one of the two femurs may be treated with the method described herein.

Optionally, a desired shape/type of implant(s), size of implant (length and/or diameter) and/or mechanical priorities are determined, for example, based on the image and/or mechanical considerations. Optionally, this leads to selecting a suitable implant kit and/or relative amounts of rods and cement, size of bag and/or lengths of components in the kit. Optionally, if stops are provided on the tools, the stops are set to a desired implant length.

Optionally, a table, software and/or stand alone calculator are provided to match up the various needs with the implant properties and/or components.

### Cutting the Skin (1004)

The skin is cut, optionally using a surgical opening, for example, an incision of 5-40 mm long. Optionally, the opening is a puncture, for example with an initial diameter of 2-10 mm and access to the bone is minimally invasive.

### Tunnel to Bone (1006)

In an exemplary embodiment of the invention, a path to the bone is formed by advancing drill 1200. Optionally or alternatively, the path is formed by a trocar and/or stylet. Optionally or alternatively, there is no need to form a path, for example, if a surgical incision is used or if bone is near the skin surface. Cannula 110 is optionally mounted on drill 1200 and its handle used for assistance. Optionally, cannula 1100 is lockable to drill 1200 using a lock (not shown), optionally activated by axial advance of cannula 110 over drill 1220 until it snap locks. Optionally or alternatively, cannula 1100 engages drill 1200 by threading thereto at proximal ends thereof.

### Open Cortex (1008)

Drill 1200 is inserted and rotated into cortex 1056, to form aperture 1058 therein. Optionally, aperture 1058 is between 2 and 5 mm in diameter, depending, for example, on the bone and/or illness characteristics, rod dimensions and/or bag thickness.

Such drilling may be, for example, manual, or using a motor to rotate and/or vibrate drill 1200.

### Engage Cortex (1010)

Cannula 1100 is advanced so that cortex cutter 1108 cuts into the cortex, widening the hole formed by drill 1200 and, once tip 1106 of cannula 1100 is advanced, engaging cannula 110 to the bone using friction and setting a gateway for accessing the bone. Optionally, cannula 110 is used to suck debris out of the wound and/or provide washing fluid therein. Optionally, drill 1200 is removed for suction. Alternatively or additionally, drill 1200 includes a suction lumen therein. Such a lumen may also be used to provide fluid and/or suction while drilling.

In some embodiments, the cortex is engaged after drilling using drill 1200 is completed, or at least after a most distal point in the bone is reached.

In some embodiments, cannula 1100 does not enter to cortex and the cortex is not engages or is engaged from its outside. This provides for a smaller aperture 1058. Alternative stabilization means may be used, for example, as described below.

### Create Channel (1012)

Drill 1200 is advanced towards the end of trochanter 1054, forming a channel in the bone. Optionally, the channel reaches, but does not contact the cortical bone at the end of the trochanter. Optionally, a distance between 3-7 mm is maintained. In an exemplary embodiment of the invention, the distance is selected to reduce damage to blood vessels and/or to reduce chance of damage to the femoral head. Different distances may apply to other bones, anatomies and/or patient conditions.

In some embodiments, a different drilling tool is used for drilling through trabecular bone.

Once drilling is completed, the channel may be washed and/or cleaned up, for example, using fluid and/or suction via cannula 1100.

FIG. 10C shows drill 1200 inside a channel 1090, formed as described herein.

### Enlarge Cavity (1014)

The channel is optionally enlarged to fit the desired size of implant, optionally an exact fit. Alternatively, an undersized or oversized cavity may be formed, the lack of fitting in one or more dimensions. In an exemplary embodiment of the invention, the channel is enlarged by cutting using cutting element 1208, while rotating and/or retracting of drill 1200. this process may be manual or motorized, with a motor optionally coordinating the axial and rotational movements to ensure that element 1208 contacts bone on all sides of the channel. Other tools as described above can be used as well.

In an alternative embodiment, the channel is enlarged into a cavity by crushing, for example, by inserting a crushing balloon or by inserting bag 1614 at a small diameter and then expanding the bag by injection of cement and/or insertion of rods.

In an exemplary embodiment of the invention, the enlarged cavity is of a diameter of between 150% and 400% of that of the (initial) channel, for example, between 200% and 350%. In some cases, some cortical bone is removed as well. In other cases the canal may curve to avoid cortical bone or may be narrow at a location where cortical bone protrudes into the canal.

FIG. 10D shows drill 1200 with extending element 1208 having formed an enlarged cavity 1092.

### Verify Cavity (1016) (Optional)

Optionally, the existence and/or geometry of the cavity is verified before the implantation process continues. Optionally, the verification is from outside the body, for example, using ultrasonic imaging or x-ray imaging. Alternatively or additionally, the verification is by imaging via cannula 110, for example inserting an optical or ultrasonic imager into the cavity. Alternatively or additionally, the verification is mechanical, for example, expanding a balloon or a winged element inside the cavity to a desired diameter and determining that the expanded element is free to move. Optionally or alternatively to verifying the diameter, the length of the cavity is determined, for example, based on positions of radio-opaque markers of drill 1200, relative to cannula 1100. Such markers are optionally located so they remain outside the bone and/or outside soft tissue. Optionally, this length is used to set the lengths of tools for the rest of the procedure and/or select the bag.

### Insert Container/Bag (1018)

Drill 1200 is optionally removed from cannula 110 and bag holder 1600 having a bag mounted thereon is inserted (e.g., for bag-based implants).

Optionally, stylet 1400 is used to guide the insertion of the bag and ensure the bag is inserted straight and not folded and/or twisted. In some embodiments, a rod 1062 is used as the stylet, as described below, for example.

In some embodiments of the invention, bag 1614 is provided pre-mounted on holder 1600. In other embodiments, the bag is mounted when used. Optionally, the bag is placed into tube 1602 and then tube 1608 is advanced until it pinches bag 1614 between the tubes. Optionally, the bag has length markings thereon which can be matched to the length shown for the cavity. In some embodiments, the bag is cut to size, for example using a cutter (e.g., an anvil cutter), not shown, provided with the kit.

In some embodiments, the bag is inflated, for example, by injection of some cement and/or by inserting a balloon, inflating the balloon and then deflating and removing the balloon.

If bag 1614 is not advanced all the way to the end of the cavity, the spacing between may be filled with cement, for example, as described below.

FIG. 10E shows bag 1614 placed in cavity 1092. An exaggerated space is shown between the distal end of bag 1614 and the edge of the cavity. Optionally, this distance is a few mm (e.g., 3, 4, 5, 6) and is later filed with cement that seeks form the bag. In other cases, such a space does not exist and/or is considerably smaller (e.g. 1-2 mm).

### Rod Insertion (1020)

Stylet 1400 (and optionally cannula 1500) are removed and a first rod 1062 is inserted, mounted on rod carrier 1800. When carrier 1800 (and/or rod 1062) is inserted all the way, piston 1808 is advanced while allowing shaft 1802 to recoil and rod 1062 is released into the bag.

In an exemplary embodiment of the invention, a plurality of rods, for example, 4-20, optionally 4-10 or about 7 are inserted, one after another. As shown below, the rods are optionally designed to slide past each other. Optionally, bag 1614 is not tightly filled with rods. Rather, the rods only fill 40%-60% or 70% of the volume of bag 1614.

Optionally, the rods are cut to size before insertion, or a plurality of rod sets of different sizes are provided. Cutting optionally uses a cutter (not shown) optionally provided with the kit.

In an exemplary embodiment of the invention, the rods are cooled prior to use. Optionally a cooling element (not shown), such as a paltier element and/or a cooling pack receptacle, being provide din the rod pusher. Optionally, such cooling offsets, at least in part, heat generated by cement setting.

FIG. 10F shows a rod 1062 already inserted into bag 1614 and a second rod 1062 being inserted.

### Cement Injection (1022)

Cannula 1500 is inserted, optionally using stylet 1400 to assist in the cannula reaching to the distal end of the implant. In other embodiments, the cannula reaches only to the proximal end of the implant. Optionally, a relatively tight seal is formed between cannula 1500 and cannula 110, to prevent cement which is injected under pressure from exiting the bone and possibly preventing proper implantation and/or damaging tissue.

In an exemplary embodiment of the invention, the cement injection fills in between the rods and also optionally leaks out of bag 1614. Optionally, leakage at a distal and/or proximal end and/or at other designated locations along the bag (e.g., adjacent resting 1078), are selected to provide anchoring sections optionally at least 10% of the implant length in length and/or optionally at least 50% of the implant diameter in radial direction. For the bag, formed of cement and/or ensure cement contact with cortical bone. Optionally, the bag includes one or more openings for such specific leakage. Optionally, one or more filaments are provided at such openings to be carried along with the cement and provide some support for the cement, optionally as a tensile element.

Optionally or alternatively, additional leakage at specific points or all along the bag, is used to form inter-digitations with trabecular bone. Various possibilities are described below.

In some embodiments of the invention, cements of multiple viscosities are injected, for example, low viscosity cement, to better leak form the bag and/or fill in between the rods and then high viscosity cement, to ensure closure of aperture 1058.

In some embodiments, no cement is injected. Optionally, rods are inserted into the bag until there is no room for more. Alternatively or additionally, an adhesive is provided, which optionally adheres the rods together.

FIG. 10G shows the insertion of a stylet 1400 to guide cement delivery cannula 1500 into the implant.

FIG. 10H shows cement cannula 1500 filling the bag with cement (stylet 1400 is removed so a cement source can be attached).

### Last Rod Insertion (1024) (Optional)

Optionally, after cement injection is completed, last rod 1068 is inserted. In an exemplary embodiment of the invention, this last rod engages aperture 1058. Optionally, rod 1068 has a flat or rounded proximal end. Optionally or alternatively, to inserting a rod, a cap is inserted. Such a cap optionally includes a proximal section of dimensions of aperture 1058 or greater (to act as a cap), for example, a cone shaped element. Optionally, the cap comprises a rod of a length shorter (e.g., 70%, 50%, 30% or greater or intermediate values) than other rods and serves to lock the implant to the cortical bone (e.g., at the proximal side thereof). Optionally, an expanding element is inserted into the implant to perform such locking Optionally, the diameter of the final rod is greater (e.g., by 50%, 80%, 100%, 150%, 200% or greater or intermediate values) than of the other rods and/or is formed of a more rigid material.

In an exemplary embodiment of the invention, rod 1068 increases the pressure inside the implant.

Optionally, backflow of cement is prevented by providing the rod and/or rod pusher with a closer fit to cannula 1100 and/or providing the rod and/or rod pusher with a o-ring type seal, which optionally moves back as rod is advanced.

Optionally, receptacle 1076 (of bag distal end) is sized small enough so only a forward tip of rod 1068, which is optionally made smaller than those of rods 1062, can fit therein. Optionally or alternatively, the last rod is positioned under x-ray control.

In an exemplary embodiment of the invention, the last rod is hollow and is used for injection of some or all of the cement into the implant. The last rod may include a forward aperture and/or one or more side apertures along its length.

FIG. 10I shows the insertion of a last rod 1068 into the implant.

### Residual Container Removal (1026)

Rod carrier 1800 is optionally maintained in place until the cement sufficiently hardens. Thereafter, the portions of bag 1614 that are outside of the bone are optionally removed. In an exemplary embodiment of the invention, the portions are removed by cutting the bag, for example using a cutter as described below. Optionally, tube 1608 has a serrated or other cutting edge and when rotated, cuts bag 1614 at location 1616. Alternative bag release methods are described below.

### End Procedure (1028)

The procedure is completed, for example, by suturing the entry hole into the body. Optionally, the leg and/or other limb can be used immediately or after a short rest, for example, to allow the cement to set.

### Anchoring of Tools

In an exemplary embodiment of the invention, the tools are anchored to the bone by cannula 110 rigidly engaging the bone (e.g., using friction). This can also provide a seal between the bone and other tissue.

Optionally or alternatively, fixation is provided by other means. In one embodiment of the invention, a framework, for example a guiding tube, or cannula 1100 (or other guiding framework, is attached to the body using external straps or other means, such as a framework, that are mounted on the body. Optionally or alternatively, the guide is attached to the body using fixation screws. Optionally or alternatively, the guide is attached to a bed/chair on which the body is supported.

A potential problem when not using cannula 1100 as described herein is that the bag may not lie/remain along the channel. In an exemplary embodiment of the invention, the bag includes at least one elastic fiber that urges the bag to remain in a straight (or other desired) configuration, which matches the channel. Optionally, the ends of the rods are machined/formed so their leading edge does not catch on the mesh of the bag.

A potential advantage of not using cannula 1100 to engage the bone is that a reduction in aperture diameter can be achieved.

### Position and Depth Determination

In some embodiments it is desirable to know the length of implant and/or distance from forward cortical bone. In an exemplary embodiment of the invention, radio-opaque markers are provided, for example, as described above with respect to FIG. 12. Optionally or alternatively, optical marks and an optical encoder reader are provided to determine the relative position of various tools.

In an exemplary embodiment of the invention, an ultrasonic sensor is provided at the tip of a tube, for example, stylet 1400 and used to determine a forward distance to cortical bone. Optionally, a user audible/visual signal is generated when the distance is correct. Optionally or alternatively, a signal is generated when the distance is too close or too far.

Optionally or alternatively, a side looking sensor is provided and used to indicate if the implant or channel/void lie close enough to cortical bone on the side.

In an exemplary embodiment of the invention, side distance is sensed by extending the side cutting tool until it touches cortical bone and noting the distance extended (e.g., on the extending manipulator or using radio-opaque markers). Optionally or alternatively, a forward extending tip (e.g., a wire) is used to sense distance to forward cortical bone.

Optionally, when the implantation process is complete (possibly before cutting the residuals), the implant is pulled back against the proximal cortical bone.

In an exemplary embodiment of the invention, drill 1200 (and/or other tools) is positioned (e.g., its position determined and optionally changed manually) using a position sensor as known in the art. For example, for rigid tools, a position sensor may be attached to the handle of the tool, with the body including a reference. Optionally, two position sensors, one on cannula 1100 and one of any tool used, indicate the relative position. Optionally, the body reference is calibrated to a previously acquired image, so that the position of the various tools can be shown overlaid on the previously acquired image. Optionally, the image is a 3D image and/or shown in 3D.

For non-rigid tools and/or some position sensor types, a probe and/or emitter may be mounted on the tool itself, for example, at a distal end of drill 1000.

Optionally or alternatively, the implant and/or tools are positioned using other means, such as x-ray (optionally two perpendicular imagers), MRI, CT, or other means known in the art.

### Alternative Void Formers

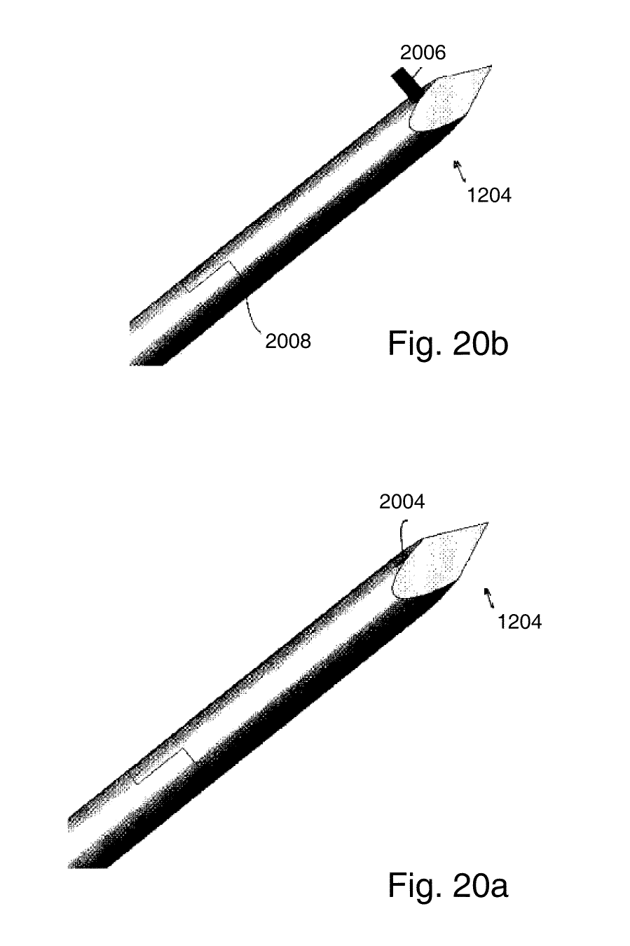

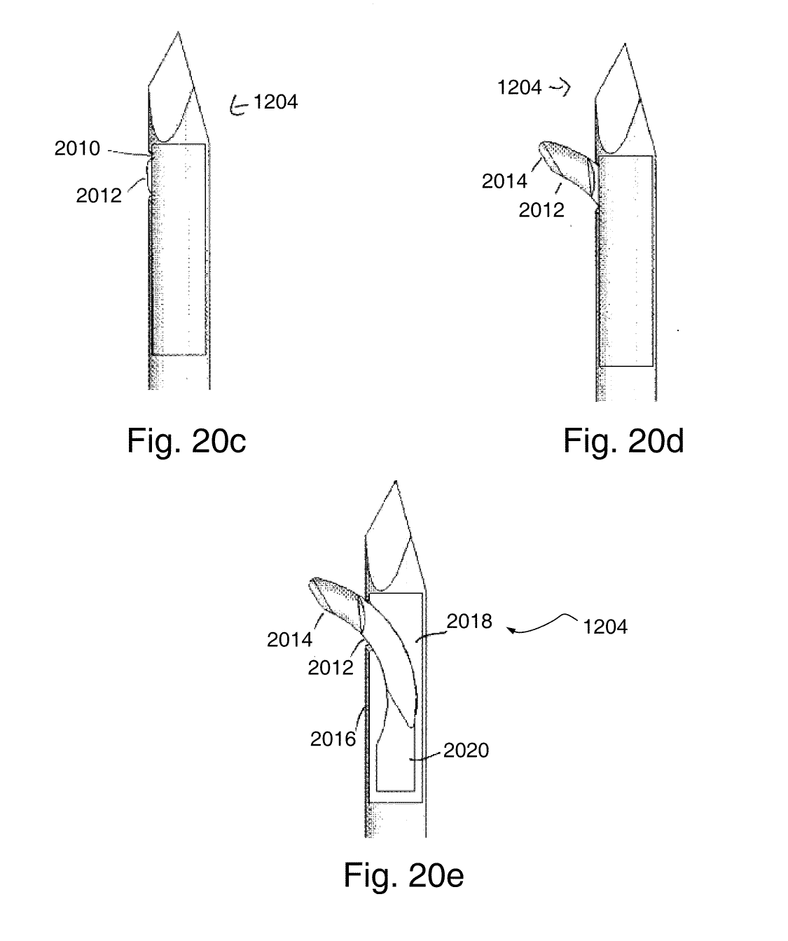

FIGS. 20A and 20B an alternative side drill extension, in accordance with an exemplary embodiment of the invention. Rather than a sharp cutting element (optionally trans-axially aligned), a wire 2006 is extended though an aperture 2004. FIG. 20A shows wire 2006 retracted and FIG. 20B shows wire 2006 extended. A potential advantage of a wire is that it may be easier to deform during delivery and/or provided at various extensions. Optionally, rotation at a relatively higher speed is used with wire-based cutting.

In an exemplary embodiment of the invention, manufacturing comprises forming a lumen in the shaft of drill 1200 and mounting a new tip (an exemplary seam 2008 shown) thereon, including the forward cutting edge and channels for guiding wire 2006.

FIG. 20C-20E illustrate another alternative side drill extension, in accordance with an exemplary embodiment of the invention. FIG. 20C shows the extension retracted. FIG. 20D shows the extension extended and FIG. 20E is a side-cross-sectional view.

In the embodiment of FIG. 20C-20E, an extension cutting element 2012 has a cutting edge 2014. Element 2012 is optionally super-elastic, so that when released (by its extension 2020 advanced distally, it curves out of an aperture 2010. Optionally, a guide such as in FIG. 12D is provided. Optionally or alternatively, the guide serves to actively shape the extension as it is extended.

In the embodiment shown, a space 2018 is provided inside the tip of drill 1200. Optionally, a shield 2016 prevent entry of bone material into volume 2018 and/or such material collects in space 2018, rather than interfere with movement of the extension. Optionally, shield 2016 is elastically disposed to seal against extension 2012.

Optionally, the embodiment of FIG. 12 uses a stainless steel cutting extension which is not markedly deformable and which may be easier to vibrationally move in and out, for example, to assist in drilling. Such motion may be provided with other embodiments described herein.

While a single extension is shown, the extension may be formed of several elements and/or may be forked. Optionally or alternatively, two or more extensions are extended from different sides of the drill tip.

Optionally, the angle of the extension is about perpendicular to the drill shaft. In other embodiments, the angle is greater or lesser, for example, being between 30-50 degrees away from the perpendicular.

In some embodiments, rather than a cutting edge, a thickening is provided at the tip of the extension. Optionally, this tip assists in cutting parallel to the drill axis. Optionally or alternatively, this tip smoothes the inner wall of the cavity. Optionally or alternatively, this tip serves to cover the exit aperture when the extension is retracted.

### Alternative Rod Pusher

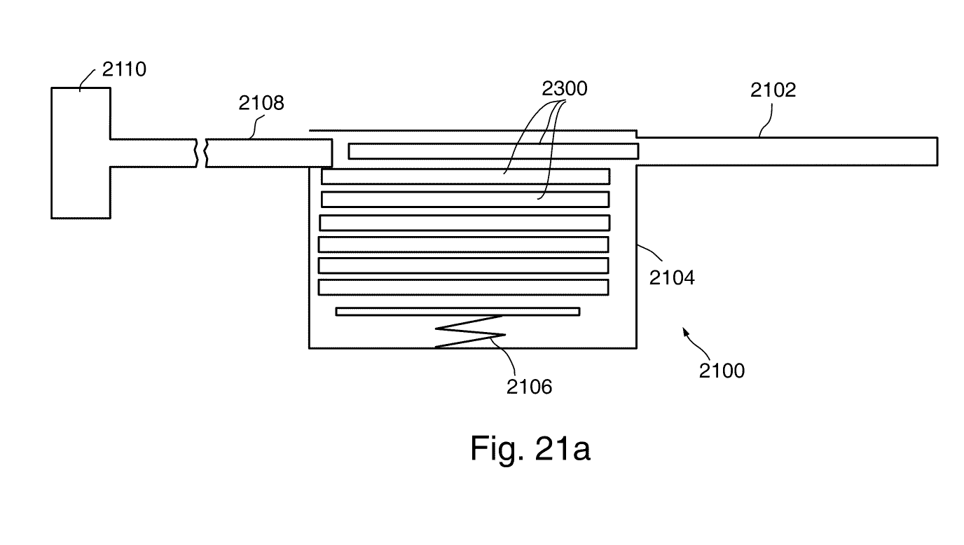

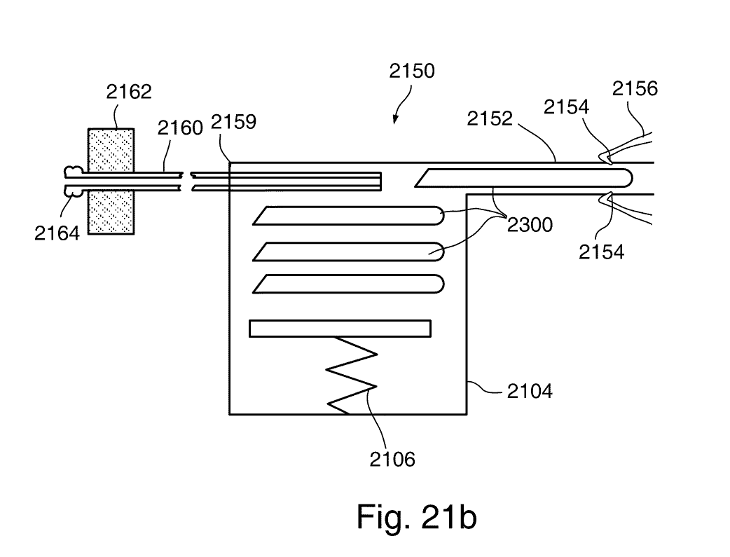

FIG. 21A-21B illustrate multi-rod carriers, in accordance with exemplary embodiments of the invention.

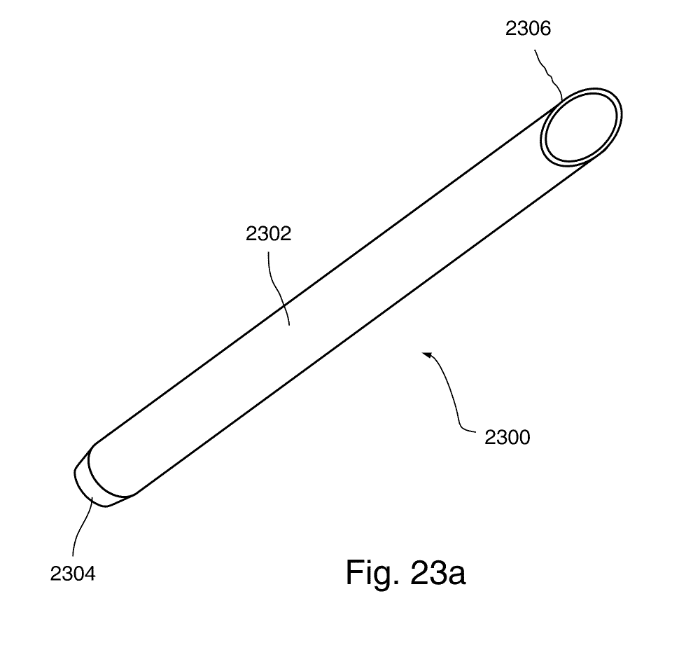

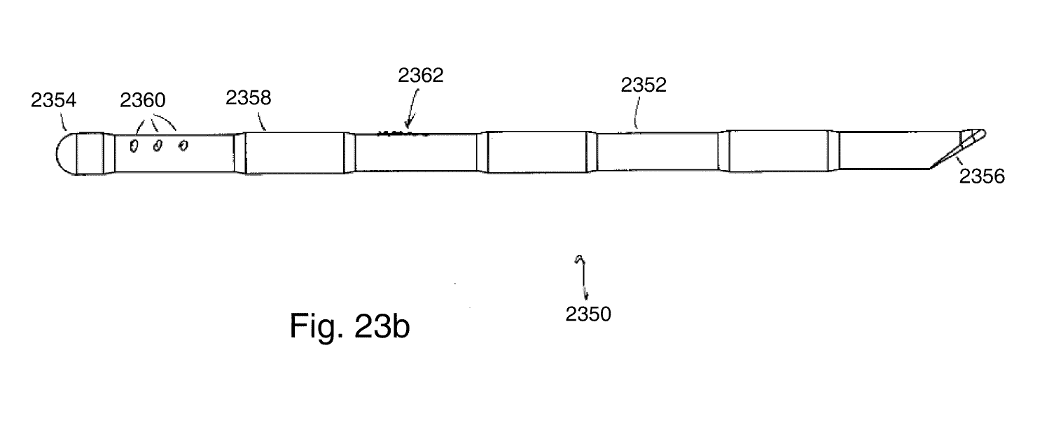

FIG. 21A shows a multi-rod device 2100, including a magazine 2104 which holds a plurality of rods 2300 (See FIG. 23).

A piston 2108, which is pulled back using optional handle 2110 allows a next rod to enter into a lumen of shaft 2102. When advanced, piston 2108 pushes the rod into location. An optional spring 2106 urges rods 2300 towards the lumen.

In this manner, a user can simply retract and advance piston 2108 for inserting rods and does not need to remove the holder form cannula 110 for each rod, nor separately mount each rod in the rod carrier.

In the embodiment of FIG. 21B, a multi-rod device 2150 uses a same shaft 2152 for rod insertion, bag holding and/or cement provision. Optionally, as shown, a bag 2156 is mounted at a mounting point 2154, for example, using one of the methods described herein, such as an outer pressure band or adhesive. Optionally or alternatively, a piston 2158 is hollow and can serve to inject cement via a lumen 2160 thereof, provided via an optional connector 2164. Optionally, there are provided a retracting means to push a rod back into the magazine, for example, a plunger opposite the magazine (not shown) and/or locking means are provided to lock the rods in the magazine (for example, a pin in the side of the magazine (not shown)).

In a particular implementation, shaft 2152 is formed to include a bone penetrating tip (e.g., in the form of a needle, optionally faceted) and the bag is provided through lumen 2160. Optionally, the bag includes a rigid ring at its neck which engages a narrowing of lumen 2160. The bag is optionally pre-mounted on a first one of the inserted rods. Optionally or alternatively, the last inserted rod is hollow and is used for cement injection. Optionally or alternatively, a first rod is hollow and used for injecting cement so as to crush surrounding trabecular bone and/or inject fluid so as to inflate the bag.

Optionally or alternatively, a same plunger is used for moving the rods as for pumping cement into lumen 2160. Optionally or alternatively, electrical control (e.g., of one or more motors) is used. Optionally or alternatively, a different switch and/or switch position is provided for each stage of operation. Optionally or alternatively, no need to manually manipulate any separate implant component exists.

Optionally, when retracted, the ring of the bag breaks and/or the needle breaks, leaving the implant in bone.

### Optional Automation

In an exemplary embodiment of the invention, the mechanism of FIG. 21B is automated, for example, using a controller (not shown) which advances rods until a certain number is inserted or a resistance felt and then injects cement, to a given amount and/or until a certain resistance is felt. Optionally, a pressure sensor (not shown) is provided between a handle 2162 of piston 2158 and a pushing motor, to sense rod resistance pressure. Resistance to cement flow and/or volume are optionally sensed by an attachment (not shown), between the cement source and lumen 2160, for example mounted on connector 2164.

Optionally, the controller is electro-mechanical. Optionally or alternatively, the controller is electrical. Optionally, a user inputs to the controller the volume of cement and number of rods to use. Optionally or alternatively, the user indicates an implant type and the rest is determined automatically.

Also noted above was the possibility that a motor mechanism (e.g., motor and gearbox) would serve to rotate and retract drill 1200 in a controlled manner so as to correctly widen the bone cavity. This can be especially useful if a small side cutting element and slow rotation are used, as manual control may be difficult in such cases.

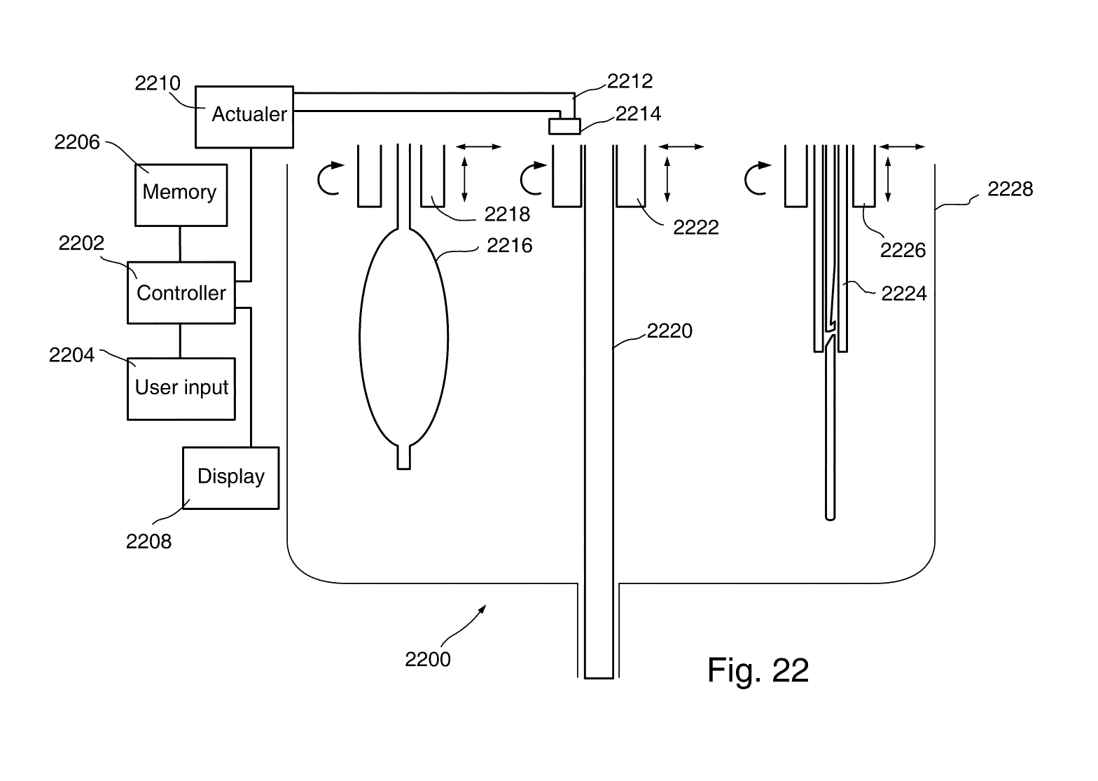

FIG. 22 illustrates a multi-tool system 2200, in accordance with an exemplary embodiment of the invention.

A first part of system 2200 is a controller/actuator section which may be useful for the embodiment of FIG. 21B as well and/or for drill control.

A controller 2202, for example a microcontroller or a computer uses an optional user input (e.g., keyboard, mouse, optional display 2208, buttons, knobs) to obtain instructions. A memory 2206 is optionally used to store instructions, settings, configuration data and/or machine commands. An actuator 2210 provides power to a selectable mechanism as described below. Optionally, the actuator comprises a plurality of motors and/or sensors. A power delivery mechanism 2212, such as a shaft couples the actuator to a tool connector 2214. In an exemplary embodiment of the invention, connector 2214 is of a type used in CNC multi-tool machines. For example, controller 2202 can select to operate a drilling guide wire tool 2220, and attaches connector 2214 to a base 2222 of the guide wire. Movements in various required directions, such as axial, rotational and optionally lateral, are optionally provided. Additional shown tools (not all of the three need to be provided) are a bag holder 2216 with a base 2218 and a rod carrier 2224 with a base 2226. In alternative embodiments, all the tools are mounted on the actuator and rotated or slid into action.

In an exemplary embodiment of the invention, the system (optionally excluding the controller portion) is enclosed in a housing 2228. In use, an initial access to the bone may be provided manually and the rest of the procedure continues under automatic control. Optionally, a user can verify each stage in the procedure, stop the procedure and/or make changes, if desired.

### Exemplary Cement

In an exemplary embodiment of the invention, the cement is injected in a viscous fluid state. Alternatively or additionally, the cement is injected in a liquid or semi-liquid state. In an exemplary embodiment of the invention, the cement viscosity and/or inclusion of particles therein is selected according to the pore sizes of bag 1614 and/or a desired degree of cement leakage.

In an exemplary embodiment of the invention, the cement has a viscosity which allows the cement to wet all the rods and the bag inner surface and/or fill the implant with less than 30%, 20%, 10%, 5% or intermediate percentages of void volumes.

In another embodiment of the invention, the filling material is a mixture of reinforced material, such as, for example, bone cement with chopped carbon fibers or bone chips and/or other fiber-reinforced cement. Optionally, said reinforced material is introduced in addition to tensile elements. Alternatively, tensile elements, such as elongated rods, are not used. In an exemplary embodiment of the invention, a “composite cements” such as Cortoss® is used.

Optionally, a non-setting cement is used, for example a bone slurry, which fixes after a relatively long period. Optionally, the bag and rods (if any) provide cohesion to the implant until such setting.

In an exemplary embodiment of the invention, a weaker cement is used, for example, a cement which releases less heat as it sets, as at least some of the strength of the implant is provided by the tensile elements.

In an exemplary embodiment of the invention, the cement used is the Disc-O-Tech Confidence/Ultra High Viscosity Bone Cement (“Confidence Cement”). The Confidence Cement is a self setting, high viscosity, radio-opaque acrylic bone cement (PMMA).

In an exemplary embodiment of the invention, biodegradable and/or bioabsorbable cements, such as Kriptonyte and calcium phosphate are used.

### Exemplary Rod and Tensile Element Design

Typical tensile elements comprise elongate objects such as filaments, monofilaments and multifilaments such as fibers, cables, threads, wires and strings. In some embodiments, a tensile element is an aggregate of a plurality of elongate objects such as yarns, braids, crochets and knits having a certain, limited, degree of axial extensibility. Typical materials from which tensile elements of embodiments of the present invention are fashioned include but are not limited to stainless steel wires, polyamide fibers (Nylons), aramid fibers (e.g., Kevlar® from E.I. du Pont de Nemours and Company and Twaron® from Teijin Twaron B.V., Arnhem, The Netherlands), polyethylene fibers (especially HMW or UHMW fibers e.g. Dyneema® from Koninklijke DSM N.V., Heerlen, The Netherlands) or Spectra®, liquid crystal polymers (e.g., celanese acetate, Vectran®), carbon fibers, and composite materials such as rods made of carbon fibers in PEEK-OPTIMA® polymer matrix (e.g., ENDOLIGN™, Invibio Biomaterial Solutions, UK), Carbon—PEKK (e.g., OXPEKK™, Oxford Performance), Carbon fibers—PMMA and DYNEEMA fibers—PMMA. Typical filaments sizes range from 10 Dtex to 600 Dtex, optionally 20 Dtex to 440 Dtex, optionally around 20 to 30 Dtex, such as 25 Dtex.

In embodiments, at least some or all of the tensile elements are bioresorbable. In embodiments, at least some or all of the cement is bioresobable

In an exemplary embodiment of the invention, the tensile elements are formed as at least one elongated rod made of a composite material, such as, for example, elongated carbon or metal fibers, embedded/“glued” together and/or encapsulated by material which serves as a matrix (for example, a polymer such as PEEK-OPTIMA®).

In an exemplary embodiment of the invention, a single composite material rod (tensile element) includes at least 40%, 50%, 60%, 70% or more or intermediate percentages by volume of longitudinal fibers (for example carbon fibers). In an exemplary embodiment of the invention, the composite material rod is manufactured prior to the procedure itself, in order to achieve good impregnation of all fibers within the polymer matrix.

In an exemplary embodiment of the invention, the rod is formed of titanium. Optionally or alternatively, the rod has tantalum or other radio-opaque material added thereto, for example, at either end, in a middle and/or in a diffuse manner. This may assist monitoring of process.

In an exemplary embodiment of the invention, each tensile element has good tensile resistance capabilities. Optionally, said tensile rod(s) is flexible, for easier insertion and manipulation, while a plurality of such rods, when situated and assembled as an implant in filling material within bone cavity, is substantially less flexible.

In an exemplary embodiment of the invention, flexible tensile elements have an elastic modulus of between 0-10 Gpa, optionally 1-5 Gpa and rigid rods have an elastic modulus of between 10-200 Gpa, optionally 10-50, or 40-120, or more.

In an exemplary embodiment of the invention, the tensile elements (rods) are straight, yet, optionally flexible enough for their manipulation within the bone while inserted. Alternatively, they are curved or bended in a desired angle and/or a desired location along the rod (e.g., “banana” shape or “J”-shape). Optionally, the rods are configured to be curved/bended during their manufacturing. Alternatively, the straight rods undergo a treatment, such as thermal treatment, to gain the desired shape.

In an exemplary embodiment of the invention, the maximal diameter of a composite material rod may be, for example, 0.1 mm, 0.5 mm, 1 mm, 1.5 mm, 2 mm, 4 mm, 5 mm or smaller or intermediate or greater diameters.

In an exemplary embodiment of the invention, the fibers within the composite material rod are having a diameter in the order of micron/s. Optionally, the rod matrix material is a second bone cement, which may be similar to- or different than the bone cement that is to be introduced into bone during the surgical procedure as described above. Optionally, the matrix material is bindable and/or crosslinkable to the bone cement introduced into bone during the surgical procedure. In an exemplary embodiment of the invention, all materials introduced into body are biocompatible.

In an exemplary embodiment of the invention, the tensile elements are smooth. Alternatively, they may be coarse and/or with protrusions in order to resist axial movements and/or to gain better adhesion/gluing within the filling material (e.g., bone cement) and/or between the elements themselves.

FIG. 23A illustrates a tensile rod 2300, in accordance with exemplary embodiments of the invention.

Rod 2300 includes a body 2302, and a distal end 2304, which is optionally rounded (to better fit between previous rods and not tear bag 1614) and a proximal end 2306 which is optionally inclined to support sliding past of ends 2304 of other rods.

In an exemplary embodiment of the invention, the diameter of the rod depends on the bone and/or implant sizes. Optionally, the rods are between 20 and 400 mm long. Optionally, for a particular usage, such as trochanter repair, the rods are between 50 and 110 mm long, optionally provided in sets. Optionally, different set sizes are provide din increments of 10 mm. in this and other embodiments, increments of different sizes for tools and components may be used, for example, 5 mm, 7 mm, 12 mm, 15 mm and/or smaller, intermediate or greater increments. In addition, the increments may be non-linear, for example, becoming larger for larger implants/bone. Optionally the rods have consecutive segments with different diameters

While solid rods are shown, in some embodiments, the rods are hollow, at least along part of their length. Optionally, the hollows are used for injecting cement through the rod. Optionally or alternatively, the rod is held (for carrier 1800) from within a hollow thereof, rather than from outside, for example, using a narrow pin or spring element.

While round cross-sections are shown, in some embodiments, other cross-sections are provided, for example, triangular, square, rectangular and hexagonal. The shape may be rotationally symmetric or not. Optionally, different rods for a same implant have different cross-sections.

In an exemplary embodiment of the invention, different rods for a same implant have other variations in proprieties, for example, type, shape, finish, length, strength, flexibility, diameter and/or material. Optionally, the selection is such as to support better interlocking of the rods and/or to prevent the rods forming a seal against cement flow in the implant.

In an exemplary embodiment of the invention, a rod can be non-uniform in cross-section, for example, have more fibers near an inside or an outside thereof.

In an exemplary embodiment of the invention, the rods are selected to be at least 30%, 50%, 70%, 80%, 90% or intermediate or greater length percentages of the final implant.

In an exemplary embodiment of the invention, the rods are shaped and/or finished to enhance cement adhesion thereto and/or to enhance cement flow inside the implant as it is being constructed.

FIG. 23B shows a rod 2350 including various exemplary means to enhance cement adhesion, in accordance with exemplary embodiments of the invention.

As shown a shaft 2352 includes a plurality of ridges 2358, which may be, for example, abrupt or gradual. For example, the profile of the rod (axially) may be sinusoidal. The pattern may be uniform along the rod or it may change, for example, monotonically or in a manner whereby the rods do or do not match each other.

Optionally or alternatively, surface treatment 2362 may be provided, for example, by sand blasting or chemical or plasma etching, to enhance cement adhesion.

Optionally or alternatively, one or more voids or passageways 2360 may be provided for cement flow therethrough.

Optionally or alternatively, one or more protrusions (not shown) may be provided on the rod, for example to enhance inter-rod locking and/or cement adhesion.

Optionally, threading and/or reverse-threading is provide don the rod. Optionally, the threading is used to carrier and/or advance the rod and/or retract the rod out of the implant, if needed.

Optionally or alternatively, the cross-sectional shape and/or orientation of the rod change along its length.

An optional rounded end 2354 and an optional inclined end 2356 are also shown.

In some embodiments, tensile elements are pre-soaked with set or non-set cement.

In an exemplary embodiment of the invention, the tensile elements are configured to be substantially non-extending. Optionally, the rods are configured so that an extension of up to 10%, 5%, 3%, 1%, 0.5%, 0.3% or smaller or intermediate values can be expected under bone stress conditions

While, in some exemplary embodiments of the invention, the rods do not anchor to bone, optionally, some anchoring ability is provided, for example, threading, sharp points and/or hooks integrally formed thereon. Optionally, such rods are used with an enclosing bag or outside of such a bag. Optionally, the tip of such a rod is pushed through a bag into surrounding bone.

### Exemplary Bag Design

In an exemplary embodiment of the invention, the bag is formed as a mesh or other porous fabric, such as a knit. Optionally or alternatively, at least some of the bag is non-porous and includes one or more opening. Optionally or alternatively, openings are provided in a porous bag. In an exemplary embodiment of the invention, the bag is generally tubular or ovoid. Alternatively, other shapes may be provided, for example, as described below. Various cross-sections can be provided, for example, circular, rectangular and/or triangular. Optionally, one or more corner reinforcement elements (e.g., a metal fiber) is provided along angles of the cross-sections. Optionally, is twisted, for example, in the form of a twisted triangle. Optionally or alternatively, the bag is spiral. In an exemplary embodiment of the invention, the bag is made of one or more of Dyneema Purity® fibers, Kevlar, aramid and/or polyethylene. Optionally or alternatively, other fibers such as described above may be used. Optionally, different fiber types and/or thickness are used for longitudinal and circumferential directions. Optionally, the weave is not parallel to the main axis of the bag. Optionally, but not necessarily, the bag is sealed at its end. Optionally, the bag is forked. One or more ribbons (e.g., in shape of a ring) of other materials, such as metal may be provided along the bag.

In an exemplary embodiment of the invention, the mesh diameter is between 2.5 and 25 mm, for example, between 5 and 15 mm, for example, 10 mm. IPA the length of the mesh bag is between 20 and 200 mm in length, for example, between 50 and 130 mm in length, for example, 100 mm in length. Optionally, an additional length of 3-15 mm may be provided for attachment onto delivery tools.

In an exemplary embodiment of the invention, the distal end of the bag is sealed, for example, by tying a knot, by a plug such as a metal mushroom-shaped plug such as shown in FIG. 10B. Optionally, such a plug is radio opaque, prevents rod penetration, holds stylet 1400 and/or holds rod 1068. Optionally or alternatively, the distal end is reinforced by the addition of one or more metal fibers (or woven or knit sections) to the mesh, optionally as part of the weaving, optionally as an embroidery.

Optionally or alternatively to a bag, a mesh or other radially expandable stent design may be used. Optionally or alternatively, the end of the bag is unsealed.

In an exemplary embodiment of the invention, pores are formed in the bag, for example, of diameter 0.1 mm. Optionally, additional pores for forming anchor sections or other large cement leaks are provided. Optionally, such additional pores are provided by inserting a needle into the mesh and optionally tying a suture. Optionally the pores are formed by the pattern of weaving or knitting of the bag.

FIG. 26 shows a pore 2610 formed in a mesh section 2600, in accordance with an exemplary embodiment of the invention. While the weave is shown to be generally open, this is for clarity. In an exemplary embodiment of the invention, the weave is tight, in some cases tight enough to prevent significant leakage and/or sweating of cement.

In the weave shown, a plurality of longitudinal fibers 2602 are woven with a plurality of circumferential fibers 2604. Optionally, at pore 2610, a circumferential fibers 2608 bends back and does not complete the circumference. Optionally or alternatively, a second circumferential fiber 2606 also folds back at pore 2610. Thus, pore exhibit only unidirectional weaving, as compared to multi-directional weaving at other places (or the weaving is multi-directional but of a lower order. Optionally, one or more longitudinal fibers 2602 lies inside the pore and has no circumferential weaving holding it (the figure shows an embodiment with no such fibers).

Pore size is optionally varied by increasing the number of fibers 2606, 2608 in the longitudinal direction, that are folded over. Optionally or alternatively, pore sizes are varied by folded over fibers 2606 and/or 2608 retracting the longitudinal fibers.

In an exemplary embodiment of the invention, a plurality of different pore sizes are provided in a bag. Optionally or alternatively, a different in seepage behavior along the bag is provided by changing a pore density along different regions (axial and/or circumferential) of the bag. For example, two regions (with seepage) can have a difference of 10%, 20%, 50%, 90%, 100%, 200%, 300% or smaller or intermediate or greater percentage in density of pores and/or total pore cross-section. In some embodiments, such percentages reflect also mesh-inherent pore sizes.

In an exemplary embodiment of the invention, a same bag may be used to provide different degrees of leakage, for example, based on the indication and/or implant properties desired. Optionally, a table is provided suggesting which mesh to and cement pair to use with which need. Optionally or alternatively, a pressure used to inject the cement may depend on the viscosity of the cement, for example, as measured directly using a sensor (not shown) or based on a mixture and setting time elapsed.

In some embodiments, the bag is straight. Alternatively, in some embodiments the bag is configured to have a curved shape along some of its length (e.g., a “J”-shape) or all of its length (e.g., a “banana” shape). In some embodiments, the bag is parallel walled. Alternatively, the walls of the bag are not parallel and are sinusoidal or include bulges. Optionally or alternatively, the bag has circumferential changes, for example, the bag being fluted (having elongate sections that extend radially or are depressed). In embodiments, the walls of a bag are made of filaments having homogenous properties. Alternatively, in embodiments, a bag is made of filaments having different properties, for example different strength and/or elasticity.

In embodiments, a container is open or is provided with a perforated wall (with perforations of up to about 0.04 mm²) allowing air to escape through the holes when a cement material is injected therein.

In an exemplary embodiment of the invention, bag elongation, if any (e.g., 1%, 3%, 5%, 10%, 20% or greater or intermediate values) and/or bag circumferential stretching, are a property of the weave used. Optionally or alternatively, such elongation or stretching is a property of the fiber used. In an exemplary embodiment of the invention, circumferential expansion is not coupled to axial expansion/retraction.

In additional exemplary embodiment of the invention, the container, and/or the tensile element fibers, and/or the tensile element matrix, and/or the bone cement are made of bioabsorbable material. Optionally, at least 20%, 50% or more by volume of the introduced materials are bio-absorbable.

### Exemplary Bag Attachment and Detachment

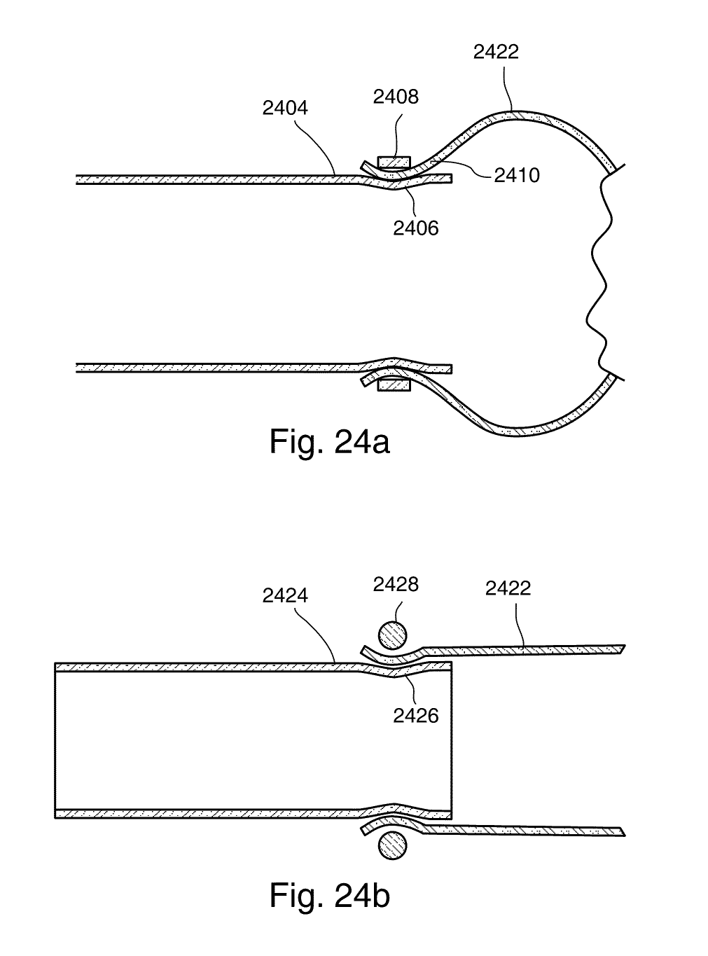

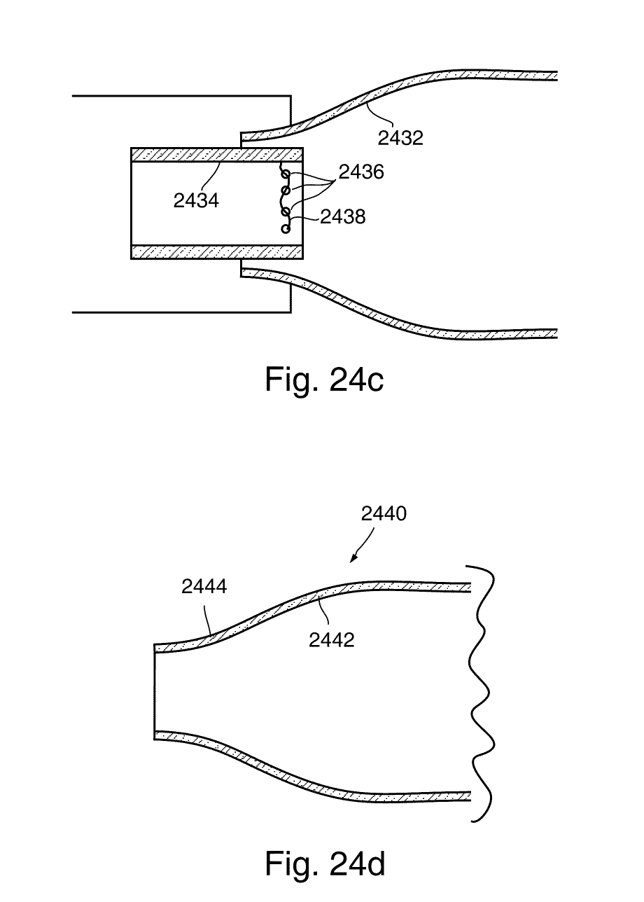

FIG. 24A-24D illustrate bag attachment methods in accordance with an exemplary embodiment of the invention.

FIG. 24A shows a bag 2402 attached to an introducer tube 2404, by a neck 2410 of the bag being pressed against a recess 2406 of the tube, by a metal band 2408. Optionally, neck 2410 is formed of a different material and/or different weave than the rest of bag 2402. Alternatively, the band is inside the tube, rather than outside.

FIG. 24B shows an embodiment where a bag 2422 is attached to a delivery tube 2424 by an elastic ring 2428 that engages the bag against an optional recess 2426.

FIG. 24C shows an embodiment, where a suture 2438 is used to attach a bag 2432 to a delivery tube 2434. Optionally, tube 2434 includes a plurality of apertures 2436, for suture 2438 to pass through. Optionally, the bag is released by cutting or releasing the suture.

FIG. 24D shows an embodiment where a bag 2440 includes a wider section 2442 and a narrower section 2444 sized to exactly fit on a delivery tube and engage it by friction. Optionally, the neck of this or other bags includes a friction enhancing layer, to reduce inadvertent slippage off the delivery tube.

In an exemplary embodiment of the invention, adhesive is used to attach the bag to the delivery tube, in addition to or instead of other means.

In some embodiments, the bag extends to outside the body and acts as a cannula or is provided within cannula 110.

Optionally, in some of the above embodiments, when sufficient axial force is applied, the bag releases from the recess. It is noted that with rods inside, the bag is typically wider than the opening in the cortical bone. Optionally or alternatively, bags 2402 and/or 2422 can be released by cutting the bands.

In an exemplary embodiment of the invention, the end of the delivery tube includes one or more apertures and/or weakening, so that when the tube is twisted, the tube breaks off and stays in the body with the bag. Optionally, the twisting serves to close proximal the end of the bag.

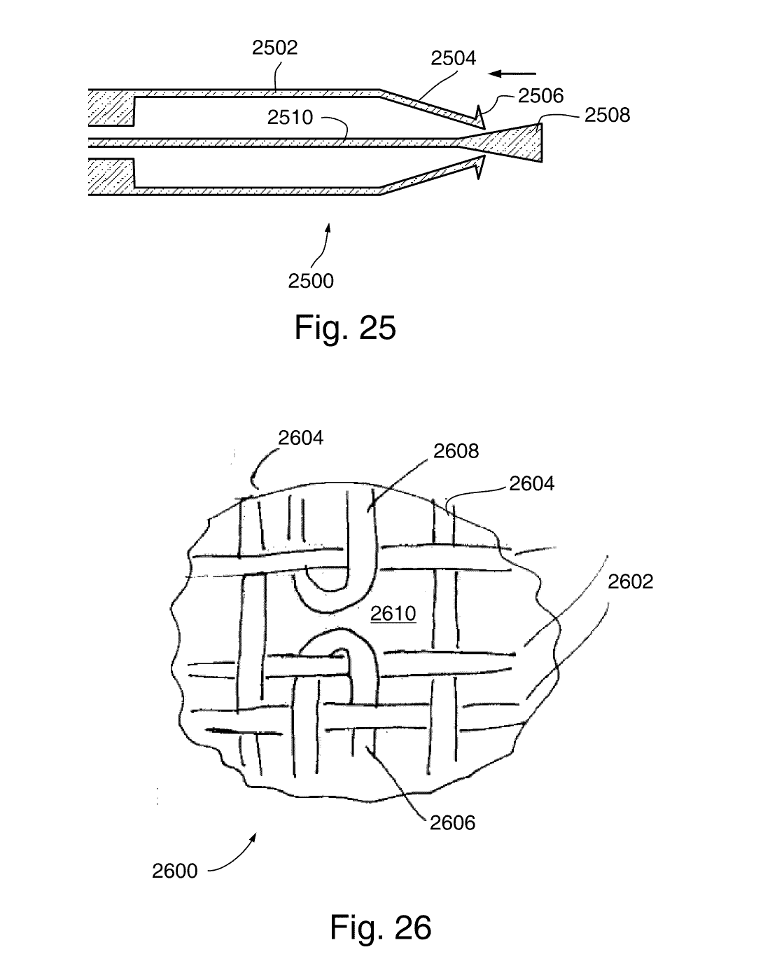

FIG. 25 illustrates a sleeve cutting tool 2500, in accordance with an exemplary embodiment of the invention.

A shaft 2502 includes a plurality of bent-in sections 2504 (may be a single slotted tube section), which include a cutting edge 2506. When a cone or other widening element 2508 is retracted (e.g., by pulling on a wire 2510), the bent-in sections extend outwards a bag section that is contacted by the cutting edge may thereby be cut. Optionally, the cutting is against an enclosing tube, such as that of cannula 1100 and/or the bag holder. The bag may be held on the outside or on the inside of the bag holder, as shown herein in various embodiments.

Alternatively or additionally, to using a cone, the cutter may be elastically or super-elastically predisposed to extend out radially. Such a cutter may be formed, for example, of stainless steel or Nitinol.

Optionally or alternatively, a non-concentric blade (e.g., a tube with an extension including a blade which is bent inwards by an enclosing tube, such as cannula 1100) is used to cut the bag. Optionally, the cutting tool is rotated to perform the cut.

### Exemplary Implant Location

In an exemplary embodiment of the invention, the bone is a leg bone (e.g., femur, tibia, fibula) an arm bone (e.g. humerus, ulna, radius), foot and hand phalanges or a clavicle. Optionally, the implant is used to repair a non-displaced fracture, such as a trochanter fracture and/or used to repair a reduced fracture.

In an exemplary embodiment of the invention, when repairing a trochanter, the implant rest son the bone (cortex) at two or three points. Optionally, multiple resting points are provided for other bones as well. In the example of the trochanter, three resting pints are optionally provided—the cortex where the implant is inserted, the inside of the femur neck, optionally midway along the femur neck, and the distal end of the implant rests on trabecular bone inside the ball of the femur.

### Exemplary Implant Mechanical Properties

In an exemplary embodiment of the invention, the implant is constructed to have certain desirable properties. It should be noted that the implant as in some of the embodiments described herein is inherently personalizable, by modifying one or more of rod type, size, shape and number, mesh type, cement type and size.

In an exemplary embodiment of the invention, one or more implant parameters are selected to match a particular need of a patient, while optionally minimizing implant size. In an exemplary embodiment of the invention the number of rods and/or diameter of selected rods is personalized for a patient. Optionally, such adaptation allows implant elasticity modulus to be selected as a function of patient condition, such as bone mineral density and/or Young modulus.

Optionally, bone properties are measured using drill 1200, or otherwise during the procedure, so that an implant personalization decision can be made on the fly. In an exemplary embodiment of the invention, bone properties are assessed by one or more of ultrasonic measurement of elastic properties, x-ray measurement of porosity and/or density, biopsy and/or analysis of residue by drilling. Optionally, difficulty in drilling and/or resistance to slow rotation of the drill with a side element extended, is used to assess a strength of the bone. Optionally, personalization can vary one or more of the above mentioned implant mechanical properties by 10%, 20%, 30%, 40%, 60% or more, up and/or down.

In an exemplary embodiment of the invention, one or more of the following considerations is used: initial give as compared to bone give, so as to allow bone to work; resistance to further give at a point where bone does not break yet; and/or total strength. Optionally or alternatively, the implant design may be determined by the direction and/or type of force expected. Optionally, implant properties are varied by changing one or more of rod diameter, rod type, rod length, rod finish, bag length and/or diameter and/or cement properties. Optionally, at least some of these properties can be varied by selecting items from a kit which includes a range of possible elements. Optionally, a table is provided to choose implant constitutes for a desired effect.

In an exemplary embodiment of the invention, the implant is used in bones in locations that experience forces addition to compression forces, for example, experiencing tension, shear and/or rotation forces.

In an exemplary embodiment of the invention, cement leakage locks the implant to bone so such forces are passed from bone to implant along substantially its entire length. Optionally, rod design and density and/or chemical bonding and/or mesh behavior assist in such functionality.

In an exemplary embodiment of the invention, the implant is configured to have an elastic modulus of between 30-40 GPa. Optionally or alternatively, the elastic modulus is less than 30 or less than 5 Gpa. Optionally or alternatively, the elastic modulus is less than 100 or less than 80 Gpa.

In an exemplary embodiment of the invention, the implant is configured to have an elastic modulus similar to that of trabecular bone, for example, within 50%, 20%, 10% or smaller or intermediate values. Optionally, different parts of the implant have different values, for example, by using rods with axially varying properties and/or using rods of a plurality of lengths.

In an exemplary embodiment of the invention, the rods are evenly distributed in the implant. Optionally or alternatively, the rods are centered around a center of the implant. Optionally or alternatively, the rods are concentrated around an outside of the implant. Optionally, the rods are moved outwards by the injection of the cement and/or insertion of stylet.

In an exemplary embodiment of the invention, the implant is formulated to have a density similar to that of surrounding bone tissue, for example, within a factor of 3, 2, 1.5, 1.3, 1.1 or intermediate or closer to 1 or even smaller than 1. Optionally, implant density is modified by suitable selection of implant constituents and/or by adding low density particles, for example, hollow spheres.

It is important to note that generally, a greater proportional amount of non-rigid tensile elements provides an implant of the present invention with greater flexibility while a greater proportional amount of cement provides less flexibility. In exemplary embodiments of the invention, an implant includes at least 20%, 30%, 45%, 60% or more or intermediate percentages by volume of longitudinal tensile elements. The relative amounts of cement to tensile elements making up a specific implant is determined by a user, for example, in accordance with one or more of the nature of the cement, the nature of the tensile elements and the desired degree of flexibility and strength of the implant. In embodiments, an implant comprises a plurality of different tensile elements having different tensile properties allowing for non-linear effects.

In an exemplary embodiment of the invention, implant size is reduced by considering the quality of bone binding by the implant and/or quality of binding of implant parts to each other. Optionally, the bone binding is enhanced by cement inter-digitations, cortical anchoring, leaning on cortex at additional point(s) and/or cement bulbs for anchoring in trabecular bone.

### Considerations for Entry Diameter

In some embodiments of the invention, the size of the bone entry hole and/or skin entry hole are important; to reduce trauma to an already weakened bone, for example.

In an exemplary embodiment of the invention, one limitation on hole size is the time it takes to insert a sufficient number of small rods and achieve a desired implant diameter. In some embodiments, the limitation is caused by the smallest diameter of the mesh bag. Optionally, the bag thickness is tradeoff with the properties of other tensile elements. In some embodiments, the limitation is that of the drilling elements, which need some rigidity for mechanical drilling and/or the time it takes to widen the cavity.

In some embodiments, cannula 1100 does not enter cortical bone. However, this presents a possible danger that the bag will not lay properly.

Various embodiments are described herein which allow a physician to trade off the various options and select a tool set and implant design which meets his needs while minimizing access pathways.

In an exemplary embodiment of the invention, the procedure is carried out using a cortical entrance diameter of between 3 and 5 mm and a cavity diameter of 10 mm. Optionally, the cavity/implant diameter is between 2 and 4 times the entry diameter, for example, ×2 ×3 ×4 ×5 ×6 or intermediate or greater ratios.

### General

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-45. (canceled)

46. A method of preventive surgery, comprising:
(a) identifying a long bone in need of strengthening; and
(b) implanting a strengthening implant in said bone through an aperture formed in the bone.

47. A method according to claim 46, wherein said bone is a hip.

48. A method according to claim 46, wherein said bone is not indicated as fractured by said identifying.

49. A method according to claim 46, wherein implanting comprises binding at least two spaced apart reinforcing elements with a binding material.

50. A method according to claim 46, wherein said strengthening implant comprising a tension-resistant element.

51. A method according to claim 46, wherein said strengthening implant comprising a bend-resistant element.

52. A method according to claim 49, comprising selecting a personalized dimension for said implant for said bone.

53. A method according to claim 46, wherein said implant is configured to rest against a cortex of said bone at one end and in a middle section thereof.

54. A method according to claim 46, wherein identifying comprises providing a patient with a problem in one limb and treating both that limb and an opposing limb, by implantation of implants therein.

55. A method of preventive surgery, comprising:
(a) identifying a long bone in need of strengthening; and
(b) building, in situ, a strengthening implant formed of a hardening material and at least one reinforcing element, which reinforcing element is not adapted to anchor in bone.

56. A method according to claim 55, wherein building comprises:
(c) forming a void in said bone; and
(d) constructing said implant in said void.

57. A method according to claim 56, wherein forming a void comprises:
(e) forming a channel; and
(f) widening said channel.

58. A method according to claim 57, wherein widening said channel comprises cutting said channel using a cutting element.

59. A method according to claim 57, wherein forming a channel comprises forming a curved channel.

60. A method according to claim 57, wherein forming a void comprises forming a plurality of voids.

61. A method according to claim 57, wherein forming a void comprises forming a void having a distal end not contacting and within about 5 mm of a cortical bone.

62. A method according to claim 57, wherein constructing said implant comprises inserting at least one tensile element into said void and filling said void using cement.

63. A method according to claim 62, wherein inserting at least one tensile element comprises inserting a bag into which said cement is provided.

64. A method according to claim 62, wherein inserting at least one tensile element comprises inserting a second bag into said bag.

65. A method according to claim 63, wherein filling said void comprises eluting at least part of said cement out of said bag to form inter-digitations.

66. A method according to claim 63, wherein filling said void comprises eluting at least part of said cement out of said bag to form at least one bulbous anchor section.

67. A method according to claim 63, wherein inserting at least one tensile element, comprises inserting a plurality of tensile elements into said bag.

68. A method according to claim 63, wherein inserting at least one tensile element, comprises inserting a tensile element having at least one end in a cortex.

69. A method according to claim 63, wherein inserting at least one tensile element, comprises inserting said bag using a tensile element.

70. A method according to claim 62, wherein inserting at least one tensile element comprises inserting a bag into which said cement is provided.

71. A method according to claim 55, wherein said method is practiced by forming a hole having a maximal diameter of less than 5 mm.

72. A method according to claim 55, wherein said method is practiced by forming a hole having a maximal diameter of less than 3 mm.

73-101. (canceled)

102. A method of constructing an implant comprising:
(a) forming an opening in cortical bone, which opening is less in diameter than a 30% of a diameter of said bone;
(b) inserting at least three non-expanding elements through said opening into said bone;
(c) completing said implant to have a diameter at least 3 times said opening diameter.

103. A method according to claim 102, wherein said non-expanding elements are elongate elements with a length to diameter ratio of greater than 3 to 1.
